SEQUENCE LISTING (141 sequences)

SEQ ID NO: 1 - DNA, 33 bp, Artificial
  primer
ggccacgcgtcgactagtacnnnnnnnnnngat

SEQ ID NO: 2 - DNA, 18 bp, Artificial
  primer
caggctctccccgtggag

SEQ ID NO: 3 - DNA, 16 bp, Artificial
  primer
aaagtgcgggtgatgc

SEQ ID NO: 4 - DNA, 20 bp, Artificial
  primer
ggccacgcgtcgactagtac

SEQ ID NO: 5 - DNA, 17 bp, Artificial
  primer
ctgcctcccagagcctg

SEQ ID NO: 6 - DNA, 23 bp, Artificial
  primer
ctgacggggtggtgcgtaacggc

SEQ ID NO: 7 - DNA, 22 bp, Artificial
  primer
cactcgtgagcgcttacagccg

SEQ ID NO: 8 - DNA, 23 bp, Artificial
  primer
gattcctctatttattcgcccgc

SEQ ID NO: 9 - DNA, 23 bp, Artificial
  primer′
ctaatcaacgaggaaaaagggac

SEQ ID NO: 10 - DNA, 24 bp, Artificial
  primer
aatcggctgaatcccacaacttac

SEQ ID NO: 11 - DNA, 24 bp, Artificial
  primer
cttctgacacatgcagtggagttg

SEQ ID NO: 12 - DNA, 26 bp, Artificial
  primer
gcgattgtacgctgaaatgaaagcac

SEQ ID NO: 13 - DNA, 24 bp, Artificial
  primer
atgccactgcatactgattaaccc

SEQ ID NO: 14 - DNA, 28 bp, Artificial
  primer
cggtgcaaatgccagataagacactaac

SEQ ID NO: 15 - DNA, 17 bp, Artificial
  primer
caggaaacagctatgac

SEQ ID NO: 16 - DNA, 25 bp, Artificial
  primer
aaacgtgaactgatgcaaggatcgc

SEQ ID NO: 17 - DNA, 25 bp, Artificial
  primer
gatcatcgctggcttgctgattggc

SEQ ID NO: 18 - DNA, 27 bp, Artificial
  primer
gatattgtgaactatcgcaaagaatcc

SEQ ID NO: 19 - DNA, 26 bp, Artificial
  primer
cgatgcacaatgcaatcttcgttgcc

SEQ ID NO: 20 - DNA, 220 bp, Artificial
  partial gene with transposon insert
caggaaacagctatgaccatgattacgccaagcttggtaccgagctcggatccactagtaacggccgccagtgtgctggaattcggcttaagccgaattctgcagatatccatcacactggcggccgctcgagcatgcatctagagggcccaattcgccctatagtgagtcgtattacaattcactggccgtcgttttacaacgtcgtgactggcaaaac

SEQ ID NO: 21 - DNA, 220 bp, Artificial
  partial gene with transposon insert
gtcctttgtcgatactggtactaatgcggttcgaaccatggctcgagcctaggtgatcattgccggcggtcacacgaccttaagccgaattcggcttaagacgtctataggtagtgtgaccgccggcgagctcgtacgtagatctcccgggttaagcgggatatcactcagcataatgttaagtgaccggcagcaaaatgttgcagcactgacccttttg

SEQ ID NO: 22 - DNA, 309 bp, Artificial
  partial gene with transposon insert
aaaaacggttaccggatccgggatcatatgacaagatgtgtatccaccttaacttaatgatttttaccaaaatcattaggggattcatcagggtcaggtctggatttaagttccatccttgatagcctcaccgccgcgcaaaaagcgacgctaacccccatttcaaatcagcaatcgtcgtttaccgctaaacttagcgcctacggtacgctgaaaagcgcgctgacgactttccagaccgccaatactgcattgtctaaagccgatcttttttccgccaccagcaccaccagcagcaccaccgcgttc

SEQ ID NO: 23 - DNA, 326 bp, Artificial
  partial gene with transposon insert
aaaaacggttaccggatccgggatcatatgacaagatgtgtatccaccttaacttaatgatttttaccaaaatcattaggggattcatcagagttaagtcccataccaaccatggacgcaatgcagaatattatgcctgggtacaaaatcatctcaaagagcatcccgcagatcgcgttgttgggtttaataagatgcctggcctggatgtttattttgccgctgatgtttgttacgccgagaaagttgcgcaagaaaaaggttttttatatcgtttaacatcacgatatccncnnnnnnngtactagtcgacgcggggccaannn

SEQ ID NO: 24 - DNA, 164 bp, Artificial
  partial gene with transposon insert
naaaaacccgccggacatcccggatccgggatcatatgacaagatgtgtatccaccttaacttaatgatttttaccaaaatcattaggggattcatcagtgttcaacctgctgcgtcgtagcccggatatatccnncnncncngnactagtcgacgcgtggcca

SEQ ID NO: 25 - DNA, 296 bp, Artificial
  partial gene with transposon insert
aacggnccgatccgggatcatatgacaagatgtgtatccaccttaacttaatgatttttaccaaaatcattaggggattcatcagagatcancctgaattccagtgatccgcttcctactgttacgccattcanngccngcaatgcggntanctntnacaaanaaggttnngtgactgttttccacagtcatggtaatgctcatgacatgagcgtctactttgtgnacccggggataataactggcaggtctacacccaggatagcagtgatccaaacagcattgcgaagacagcg

SEQ ID NO: 26 - DNA, 317 bp, Artificial
  partial gene with transposon insert
aaacggttaccggatccgggatcatatgacaagatgtgtatccaccttaacttaatgatttttaccaaaatcattaggggattcatcagtgcttacacctggcagccgatctatcaaacatgcgggcggttaatggccgtggagctattaacggtggtcacgcatcccttgaacccttcgcaacgcctgccgccggatcgctattttactgaaatcaccgtcagccatcggatggaggttgtgaaagagcagattgatttgctggcgcaaaaagccgacttctttatagagcacggcctgctggcatcggtcaatat

SEQ ID NO: 27 - DNA, 371 bp, Artificial
  partial gene with transposon insert
aaacggttaccggatccgggatcatatgacaagatgtgtatccaccttaacttaatgatttttaccaaaatcattaggggattcatcagcttcaagcgccagtttcaacatgcggttgatgacgcgctgttcggtatgggtaaactcgcgaccttccactttggtcgggaagcgtccatcgccgccaaacaggttatccacggcgataaacaccagactcggtgagaacaccaccagcccagtgccgcgcagcggtttcagatggataaggttcaggttggtcggcaccggcaggttgcgggcaaattcatggtacggctgaatgcggatggccccgacggttatatccngncnnnnngtactagtcgacg

SEQ ID NO: 28 - DNA, 374 bp, Artificial
  partial gene with transposon insert
aaacggttaccggatccgggatcatatgacaagatgtgtatccaccttaacttaatgatttttaccaaaatcattaggggattcatcagtatccagccccaacgtatggaaagtggagatcatcagcccacgcgcctctttgcgccccagcgtctgccctacacgctctttcatctcgcgcgctgctttattagtaaaggtcaccgccgcaatgtgccgcgcctgataaccgcaaccgcggatcagatgggcgattttattggtgataacacgagttttaccggaacccgcgcccgccagcaccaggcaggggccggtaacgaattcgacagcttgttgttggccggggtttagacgcataggtgtattgctca

SEQ ID NO: 29 - DNA, 311 bp, Artificial
  partial gene with transposon insert
cggnccggatccgggatcatatgacaagatgtgtatccaccttaacttaatgatttttaccaaaatcattaggggattcatcagtggtacgcatctacataaattttgtcgatcaccggtgacataataaaaaaggtcaaaaacagtgccagccccagcaatacctggttaggtggcgcgcggagggtgttcccagcgcgttacgcaataaaccaaaaacaatgatgatgcgggtgaaactggtcatcatcagtaaaattgccggaataaacgtcaacgaggtgatgaacaccagcgtctgcaccgggagc

SEQ ID NO: 30 - DNA, 398 bp, Artificial
  partial gene with transposon insert
nncnnaacanacggnnccggccnggatcatatgacaagatgtgtatccaccttaacttaatgatttttaccaaaatcattaggggattcatcagcggcgagctggcgcagaaaatcatcgacgagatgttcctgttcgagaatctggtggatntcgccaatcgcngtactagtcgacgngtggccaaantggattccnaatcgctgntgatcgcgctgaaaggagccgagcngtcactgtgcnagaanttcttgcncnatatgtcgcnncgtgccgccnatatcccncnccnaccgtacccctngnacgnnnnaccngnaccccnntncggncaagnatgnnannanccngatanancagnncantnctnngatncacnnnatannanngncgccnac

SEQ ID NO: 31 - DNA, 312 bp, Artificial
  partial gene with transposon insert
aaacggttaccggatccgggatcatatgacaagatgtgtatccaccttaacttaatgatttttaccaaaatcattaggggattcatcagggtcaggcatgctctcgcattctggaagcgttaaaaaataatcggatatcactatgagttttgcgaccatttctgttatcggactgggttatatcgggctgccaacngctacncgacncgcgccncggcaaaaacaggtaattggtgtcgatatcaaccaacatgcggttgataccatcaatcgtggccaaatccatatcccccccccccgtactagtcgacg

SEQ ID NO: 32 - DNA, 250 bp, Artificial
  partial gene with transposon insert
cggnccggatccgggatcatatgacaagatgtgtatccaccttaacttaatgatttttaccaaaatcattaggggattcatcagtgccgcgcacggcaaatccgaatgcaaaaatcgcactaatctcaaaagtatacccgatgcgtagccataccgttgctgcagaagggggctccgccgctgtcgcgcaggatcatgacagcttcgaatatccccnnncccccgtactagtcgacgccgtgaanannnn

SEQ ID NO: 33 - DNA, 316 bp, Artificial
  partial gene with transposon insert
aacgtaccggatccgggatcatatgacaagatgtgtatccaccttaacttaatgatttttaccaaaatcattaggggattcatcagggcgcagccgctgcgtgagtttatgctgcgtcagacccgtgaggcagatttagggttgtttgccagactggcgaatatcggcccgttgcgtnctactcnacncgtgccnatgcgcattttgctnccngcctacgtgaccagcgagttgaaaaccgcatttcagatacggcttcacagattttcatcccttttttgattatcgacctggtgatagccagcgtgttgatggc

SEQ ID NO: 34 - DNA, 276 bp, Artificial
  partial gene with transposon insert
atgaaaacgnnccggatccgggatcatatgacaagatgtgtatccaccttaacttaatgatttttaccaaaatcattaggggattcatcagtactcaacttccagcggattaatcgccttccaggcgtcgctatagccttcaagcgccagtttcaacatgcggttgatgacgcgctgttcggtatgggtaaactcgcgaccttccactttggtcgggaagcgtccatcgccgccaaacaggttatccacggcgataaacaccagactcggtgagaa

SEQ ID NO: 35 - DNA, 332 bp, Artificial
  partial gene with transposon insert
cgtaccggatccgggatcatatgacaagatgtgtatccaccttaacttaatgatttttaccaaaatcattaggggattcatcagggcgcagcccgctgcgtgagtttatgctgcgtcagacccgtgaggcagatttagggttgtttgccagactggcgaataccggcccgttgcagggacctgaagccgtgccgatgcgcattttgctcccggcctacgtgaccagcgagttgaaaaccgcatttcagataggcttcacgattttcatcccttttttgattatcgacctggtgatagccagcgtgttgatggcattggggatgatgatggtt

SEQ ID NO: 36 - DNA, 215 bp, Artificial
  partial gene with transposon insert
ngnnnatacgnccggatccgggatcatatgacaagatgtgtatccaccttaacttaatgatttttaccaaaatcattaggggattcatcagtattaagcgtgaatgatgccagagcgcaaagcgttcaatggtttgagtaaggggcaaaacaggcgggatttagggcttttgctgccacatatccnnnnnnnnngtactagtcgacgcgtggcca

SEQ ID NO: 37 - DNA, 394 bp, Artificial
  partial gene with transposon insert
aaacggnccggatccgggatcatatgacaagatgtgtatccaccttaacttaatgatttttaccaaaatcattaggggattcatcagtatgtgtaccgtgtattggcgctaagngtgngaagctttttttccaantaccatgcttgccnnatgaccataangttgcgatatnttcattccgtgcatgcaaacancgtaccnncagcgccaccatncaactgatgcgtcngantaatgaccaggttntanttattctntcgcccgagcctcatcancnaangctcnctttntnnncggnannnnnattttncccngtctnnntnttgnttnantnnnttacgcggcnacnnattngttntggtcntacgngcnnnataacngcnnctcncnnncc

SEQ ID NO: 38 - DNA, 450 bp, Artificial
  partial gene with transposon insert
cgncggatccgggatcatatgacaagatgtgtatccaccttaacttaatgatttttaccaaaatcattaggggattcatcagcgtttcgcccctattacagactcacaaccacatgatgaccgaatatatagtggagacgtttagatgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgatatcccnccccccng

SEQ ID NO: 39 - DNA, 300 bp, Artificial
  partial gene with transposon insert
taccggatccgggatatatgacaagatgtgtatccaccttaacttaatgatttttaccaaaatcattaggggattcatcagcttcaagcgccagtttcaacatgcggttgatgacgcgctgttcggtatgggtaaactcgcgaccttccactttggtcgggaagcgtccatcgccgccaaacaggttatccacggcgataaacaccagactcggtgagaacaccaccagcccagtgccgcgcagcggtttcagatggataaggttcaggttggtcggcaccggcaggttgcgggcaaatt

SEQ ID NO: 40 - DNA, 300 bp, Artificial
  partial gene with transposon insert
nncgtncgggatattgacaagatgtgtatccaccttaacttaatgatttttaccaaaatcattaggggattcatcagcgcttgaaggctatagcgacgcctggaaggcgattaatccgctggaagttgagtacgtgcgttcggaaatgcaggtgaaatttaccaatatcaccacctcgccgaacgacattgtggttaacacgccgttccatgtggagattggcaacctgaccggcgaatttaatatctgcctgccattcagcatgatcgagccgctacgggaattgttggttaacccgcc

SEQ ID NO: 41 - DNA, 250 bp, Artificial
  partial gene with transposon insert
anccgatccgggatatatgacaagatgtgtatccaccttaacttaatgatttttaccaaaatcattaggggattcatcagcgcttgaaggctatagcgacgcctggaaggcgattaatccgctggaagttgagtacgtgcgttcggaaatgcaggtgaaatttaccaatatcaccacctcgccgaacgacattgtggttaacacgccgttccatgtggagattggcaacctgaccggcgaatttaatatc

SEQ ID NO: 42 - DNA, 260 bp, Artificial
  partial gene with transposon insert
cgnccggatccgggatcatatgacaagatgtgtatccccttaacttaatgatttttaccaaaatcattaggggattcatcagcaccctgcattcgcaggagcggagaaacatccgccggagcggcaatcaccactgagcgtgcacgcccttcggcaccgaggatgttctcaataaaatcttttacttcgcgcccacgttcaccaatcaaacccacgacaatgatatccnnnnnnnnngtactagtcgacgcgtggccaat

SEQ ID NO: 43 - DNA, 378 bp, Artificial
  partial gene with transposon insert
tangaaaaacgtaccggatccgggatcatatgacaagatgtgtatccaccttaacttaatgatttttaccaaaatcattaggggattcatcagcgctccagcccctggagagacgcttnccagtccgcgcgttcaaaaatgccttgttgcaaatagccctccagctgcggccacagggcgatggctttatcgagcatcggatcgctgcctttggcatacgcgccgacgctaaccagatcgcggttacgctgaaaactcgacaacagctgtttgaaggtgcgcactcgcgcgtaatgttgctcactgatcaacgccgtcattgcgcggctgatcgacgcttcaatatccnnnnncnnngtactagtcgacgcgtggcca

SEQ ID NO: 44 - DNA, 207 bp, Artificial
  partial gene with transposon insert
agcncgccggacntcccggatccgggatcatatgacaagatgtgtatccaccttaacttaatgatttttaccaaaatcattaggggattcatcagagtcgctagtggaaaagccatttcgaaaaatcctggtcataaagatgcgatatccccccaccgcgtactagtcgacgcgtggccananannnnnnncggcannnccncccnt

SEQ ID NO: 45 - DNA, 351 bp, Artificial
  partial gene with transposon insert
atacccgatccgggatcatatgacaagatgtgtatccaccttaacttaatgatttttaccaaaatcattaggggattcatcagcgcattgcatcaacagttgccgcacggggccaccatgttcgggtatatacacagtcgtgggaaggcgattgcccgaaagcatttgagcttattcangngccagttaagtcccntaccaaccatggacgcaatgcagaatattatgcctgggtacaaaatcatntnaaacagcntcccncaggatctgtgtcgnngngttcaattattatnccctcgncnggattatcttatttgcctgctnaatgtcngtcttctnattcctaaatnt

SEQ ID NO: 46 - DNA, 276 bp, Artificial
  partial gene with transposon insert
ngaaaacggnccggatccgggatcatatgacaagatgtgtatccaccttaacttaatgatttttaccaaaatcattaggggattcatcagcgcttgaaggctatagcgacgcctggaaggcgattaatccgctggaagttgagtacgtgcgttcggaaatgcaggtgaaatttaccaatatcaccacctcgccgaacgacattgtggttaacacgccgttccatgtggagattggcaacctgaccggcgaatttaatatctgcctgccattcagca

SEQ ID NO: 47 - DNA, 300 bp, Artificial
  partial gene with transposon insert
cncgatccgggatcattgacaagatgtgtatccaccttaacttaatgatttttaccaaaatcattaggggattcatcagattcaggtgcgtaataagctggtggcggcgtatcaggaagtgatgagcatgcaggtgtaggttttgtaacctgttgttaattacatccgatctcaccgcgtggggcatggatggggcaaactcactcaatttctggtttaaaatggatacctgatcctgaacgttttctgccatccacgatccgtatacgcgataacccccctgaacatcagagcgtctca

SEQ ID NO: 48 - DNA, 337 bp, Artificial
  partial gene with transposon insert
naaaacgnccggatccgggatcatatgacaagatgtgtatccaccttaacttaatgatttttaccaaaatcattaggggattcatcaggggttaataccagtttttcgcgctccggcaacgtttcgatggcttccatcacccgctggcgcagattactgtccagtagttgttgtagcgggttttctcgctgatgatcatcagtaaccagttcgatgctatcgccgtgctcttcgcgccactcatcgtaggagaagagctggctgttattggtgtcgagcaacatttggcgataatcggcaatatcnnccccccccgtactagtcgacgcgtggccaa

SEQ ID NO: 49 - DNA, 351 bp, Artificial
  partial gene with transposon insert
atacncgaccgggatcatatgacaagatgtgtatccaccttaacttaatgatttttaccaaaatcattaggggattcatcagagtttagctgctgccaacgcagcctgacaaagtgcgggatcgccaccttcaggtttgatatccagtaacataccatcgggtgccagttttatgcgcagcgtacaggttttgcctgcataggacgatgcgtcatagaacttactttcgatagcagatttaatctgcccggcatagttattgatatccnccccccnngtactagtcgacgcgtggccanntattcngatatcncnccngccngtctantcccncgtggncatatctgatnc

SEQ ID NO: 50 - DNA, 172 bp, Artificial
  partial gene with transposon insert
anngnaanagcncgccggacatcccggatccgggatcatatgcacaagatgtgtatccaccttaacttaatgatttttaccaaaatcattaggggattcatcagagtcttgntccactttgccaatancgccggccntagcnctagncgncgcacgnncagacgcgtggcca

SEQ ID NO: 51 - DNA, 270 bp, Artificial
  partial gene with transposon insert
cgntaccggaccgggatcatatgacaagatgtgtatccaccttaacttaatgatttttaccaaaatcattaggggattcatcagtgctcggctcctttcagcgcgatcaacagcgattcggaatccacttcctgcaacagacgctgaatgctgcgatcgtcgacatccaccagattctcgaacaggaacatctcgtcgatgattttctgcgccagctcgccgtcgaattcacgcacggcggtaataacggcttcttcctgctgagttttc

SEQ ID NO: 52 - DNA, 390 bp, Artificial
  partial gene with transposon insert
ggaaaaaacgtccggatccgggatcatatgacaagatgtgtatccccttaacttaatgatttttaccaaaatcattaggggattcatcagggttgggtaccctttgtgttgggcaaaaccatattgcgggaaaacaatatccagcgccgccnttnnngcgtcacgcgtnactttcgccaggatagacgcggcactgatttccggtacgcggctatcgcctttcaccacagccatcgcaggcatcggtaatttcgggcagcggttaccatcaatcaacacatattccggcgcaatatgcagcccagcgacggcacgctgcatcgccagcatggtcgcatgaagaatgttcagctcgtcgatatccnnnnnncncgtactagtcgacgcgtg

SEQ ID NO: 53 - DNA, 226 bp, Artificial
  partial gene with transposon insert
tangaaaaaaacgttccggatccgggatcatatgacaagatgtgtatccaccttaacttaatgatttttaccaaaatcattaggggattcatcagggttgggtaccctttgtgttgggcaaaaccatattgcgggaaaacaatatccnccncccnngtactagtcgacgcgtggccaaatatnnnnatatnnnnnnnnnnnnnnctannnnncncgnggnnannnn

SEQ ID NO: 54 - DNA, 226 bp, Artificial
  partial gene with transposon insert
nnngnaaagcccgccggacntcccggatccgggatcatatgacaagatgtgtatccaccttaacttaatgatttttaccaaaatcattaggggattcatcagagtcgctagtggaaaagccatttcgaaaaatcctggtcataaagatgcgatatccngnnnccnngtactagtcgacgcgtggccaaaannncnnnnnnnannncnatnctngcncccnnccanc

SEQ ID NO: 55 - DNA, 425 bp, Artificial
  partial gene with transposon insert
cgaccggatccgggatcatatgacaagatgtgtatccaccttaacttaatgatttttaccaaaatcattaggggattcatcagcactgaatctggcattaggtttacttaatcgtatggccccgcaattatccatttttgttattggatttccattaactctgactgtcggcatctctttaatggcggcattaatgccgttaattgcacctttttgcgaacatttattcagtgaaatttttaatttgctggctgatattattagtgaattgccattaatataattccgtaacgtttatcatgttatcctaaggattatccgaaaaataatacctacgaacatcttccaggatactcctgcagcgaaatatttgttttaagctcactcacatatcccncccnccngtactagtcgacgcgtggcca

SEQ ID NO: 56 - DNA, 176 bp, Artificial
  partial gene with transposon insert
aacnaaacggntncggcatcgggatcatatgacaagatgtgtatccaccttaacttaatgatttttaccaaaatcattaggggattcatcagcgctgcgcatacttatgccatatccnncnngnnggtactagtcgacgcgtggccanattnnnnnatcnncnnnnnnggggcnnn

SEQ ID NO: 57 - DNA, 355 bp, Artificial
  partial gene with transposon insert
aaacgaccggaccgggatcatatgacaagatgtgtatccaccttaacttaatgatttttaccaaaatcattaggggattcatcagggggcaagttgcttttgggcgtctcgtagcctttcccggctaaagattgagcttgctttgcgacatcatcaatactgaaagcccagcttgaagatgtatacagggttaacattactgcagcactcaaccaacgcattttcattatttgtagcttatgtttcataattagtaaggcacttccccctttngtgtgcttatatccctnngntnncntnctagtcnacncgtggccactnctatccngnnntccccgntnngcagtantcagac

SEQ ID NO: 58 - DNA, 380 bp, Artificial
  partial gene with transposon insert
gatcatatgacaagatgtgtatccaccttaacttaatgatttttaccaaaatcattaggggattcatcagggctgcgcccctttttccagcgcctcctgcattgatattttctcttcgctgaatggctggtacgcatctacataaattttgtcgatcaccggtgacataataaaaaaggtcaaaaacagtgccagccccagcaatacctggttaggtggcgcggagggtgttcccagcgcgttacgcaataaaccaaaaacaatgatgatgcgggtgaaactggtcatcatcagtaaaattgccggaataaacgtcaacgaggtgatgaacaccagcgtctgcaccgggagcgaccagctttgtccaccgccaggcagcg

SEQ ID NO: 59 - DNA, 449 bp, Artificial
  partial gene with transposon insert
natacgnccggatccgggatcatatgacaagatgtgtatccccttaacttaatgatttttaccaaaatcattaggggattcatcagggtgcaacctgtatcacacccgatgaaagattaattagtcaagattatgatatctttttaacggataatccgtctaatcttactgcctctggcttgcttttaagcgatgatgagtctggcgtacgggaaattgggcctggtcaattgtgcgtcaacttcaatatgagcaacgctatgcaggaagcggtcttacaattaattgaagtgcaactggcgcaggaagaggtgacagaatcgcctctgggcggagatgaaaatgcgcaactccatgccagcggctattatgcgctctttgtagacacagtaccggatgatgttaagaggctgtatactgaagcagcaaccagtgactttgctgcgtta

SEQ ID NO: 60 - DNA, 450 bp, Artificial
  partial gene with transposon insert
annnncgatccgggatcatatgacaagatgtgtatccaccttaacttaatgatttttaccanaatcattaggggattnatcagctnaatgcatgcncnaccattgcctcngctgtttgcaaccgngtaaggngcattcatntgcatatgttgctgcnancaatctggctgagnagacaagcncactcccatganacgcatcgcgcattattntacgtgaaancggatnnaanggntggntaaaccnanganccnncgccgantatnnttccnctgncannctnanntngnctngnacngannncnanncnacncctctttntnnnnttccgntnnngnngnnnnnnntngtnntccnncctgtntncanntnncnnngntcntncncccntctntccantgccanttgtnncnaggtncgatntctgcngaccnacnnnntagnanccnn

SEQ ID NO: 61 - DNA, 212 bp, Artificial
  partial gene with transposon insert
gnaaancccgccggacatcccggatccgggatcatatgacaagatgtgtatccaccttaacttaatggatttttaccaaaatcattaggggattcatcaggcggtagcgtgcttttttctgcttaacttaaccagacaatcacacaaaagagtcgctagtggaaaagccatttcgaaaaatcctggtcataaagatgcgatatccnnnnnnn

SEQ ID NO: 62 - DNA, 333 bp, Artificial
  partial gene with transposon insert
aacgctnccgatccggatnatatnaacagatntgtatncaccttatcttaatganattttaccanaatcatattggggatatcatatangggctgccccctttttccagcccctactgcattgatattttntcttcgntgnatggctggtacgcatctacataantnttgacgatcaccggtgacntaataaaanaggncnnanncantgccantcccagcaatncntggttnngtggngcggacggtgctnccatngcnngacncacnnaaccnannncnatgangntnccncnganantgganatcatcctgcaanncnacngnatncnna

SEQ ID NO: 63 - DNA, 284 bp, Artificial
  partial gene with transposon insert
cgnccggatccgggatcatatgacaagatgtgtatccaccttaacttaatgatttttaccaaaatcattaggggattcatcagcgatnanctgcaataagcagaaccncctttttggnttaatatgtccttacaaatagaaatgggtctttacacttatctaagatttttcctatatcnncncnccnngtactagtcgacgcgtggccatttatnnnnnatntcctnntngtctcnngnncnncncgcggccncancnnnatatnnntnnnncnctncactctn

SEQ ID NO: 64 - DNA, 420 bp, Artificial
  partial gene with transposon insert
aacccgatccgggatcatatgacaagatgtgtatccaccttaacttaatgatttttaccaaaatcattaggggattcatcagggctgggttggcaatggtgtctacgtttctggtgtgcagcgtgagtatgatgcgtttattaccaaccagttacgtgcggcgcagacgcaaagtagcggtctgactgcccgctatgagcagatgtcgaaaatcgacaatatgctctccaccagtacctcttcgctggcaacacagatgcaggatttcttcaccagcctgcaaacgctggtgagtaacgcggaagacccggcagcgcgccaggcgctgattgggaaatcagaaggattggtgaatcagtttaaaaccaccgatcaatatcnncnnnncccgtactagtcgacgcgtggccanannatnct

SEQ ID NO: 65 - DNA, 283 bp, Artificial
  partial gene with transposon insert
ataccngatccggntcatatgacaagatgtgtatccccttaacttaatgatttttaccaaaatcattaggggattcatcaggcttacgcctgcttttgcgttgttgatggcatcacggatcccgcttaacgatgagttagccgcgctgatatcaatggtnatcgtactantcgacgcgtggccatgaatggtgagtttactgtcgctggtggcgatcgccgttttcatatcgncnnttncngtactagtcgacgcgtggccaaattnntntnanaaaaattcn

SEQ ID NO: 66 - DNA, 334 bp, Artificial
  partial gene with transposon insert
aacggttaccggatccgggatcatatgacaagatgtgtatccaccttaacttaatgatttttaccaaaatcattaggggattcatcagcgcgcgatatcgattttgccagtgaacttaaaaaagtcatgcaacgtggacgggatgcaaccagtgtggttgcactgacgatgacctcaacgcaacacattccggcgcaggcgctgacgcctcctaccgcagaactgcaataccgtattccggaccagccttcgcttgacggtaataccgtcgatatggatcgcgaacgcacccagtttgccgataacagcctgcaataccagatgagccttagcg

SEQ ID NO: 67 - DNA, 272 bp, Artificial
  partial gene with transposon insert
cgntaccggatccgggatcatatgacaagatgtgtatccaccttaacttaatgatttttaccaaaatcattaggggattcatcagcacgacgcgttgctgcgctttgtcgtcagcggcaacctgatgcgaatgccagtagctgtaacctgcgctggcacaggccgcgagggtaatgaataccagaatcgggatccaaagcgatcgcttgcttttcttgcttatcgcgtaatcagtcatgtgttgcggtcttcctgtgtcgctactgcttatc

SEQ ID NO: 68 - DNA, 394 bp, Artificial
  partial gene with transposon insert
aaacgaccggatccgggatcatatgacaagatgtgtatccaccttaacttaatgatttttaccaaaatcattaggggattcatcagcgctaagtgtgtgaagctttttttccaaataccatgctgccgatgaccataaagttgcgatattttcatccgtgcaggtaaacaacgtaccagcagcgccaccatccactgatccgtaagattaatgaccaggtcataattattcgcacgcagagnttttatcaacnaaagcacatttttaatttnatcgaaagttcccncncctttattgcttancccatagagcgcantnntttccgggtttncanacaaaatntggatggtgncctgncnaagcnncangtctanntnngctntatgagaatctg

SEQ ID NO: 69 - DNA, 392 bp, Artificial
  partial gene with transposon insert
aaacgaccggatccgggatcatatgacaagatgtgtatccaccttaacttaatgatttttaccaaaatcattaggggattcatcagagttttacccgtttcggtacgctgcgttcgctcagaatcggcgcggtggagatcatcgccagctcncgcantnacggncagaantacaggtttaaccaggatagccattgttngcttgccacctgcancatagtacggttacccnatgatttactngnaggttagtgaacaangtgcggncagtnattcancaacacattnngcatgntctgtctnggcanntattttggtgatnaananggccgatnnttttncgantnnccgnnntgggntncttnttcatcnagtnncnnatgggcgngtatn

SEQ ID NO: 70 - DNA, 482 bp, Artificial
  partial gene with transposon insert
tacgnccgatccgggatcatatgacaagatgtgtatccaccttaacttaatgatttttaccaaaatcattaggggattcatcagggctgcgcccctttttccagcgcctcctgcattgatattttctcttcgctgaatggctggtacgcatctacataaattttgtcgatcaccggtgacataataaaaaaggtcaaaaacagtgccagccccagcaatacctggttaggtggcgcggagggtgttcccagcgcgttacgcaataaaccaaaaacaatgatgatgcgggtgaaactggtcatcatcagtaaaattgccggaataaacgtcaacgaggtgatgaacaccagcgtctgcaccgggagcgaccagctttgtccaccgccaggcagcggctggctggtgatacccggcagttgcgcgaaggcgaggggcgtaatcagccagagaaggacaggtgcgacagacaataaacgacgcat

SEQ ID NO: 71 - DNA, 225 bp, Artificial
  partial gene with transposon insert
gngnaaaaacgtnccggatccgggatcatatgacaagatgtgtatccaccttaacttaatgatttttaccaaaatcattaggggattcatcaggatggtgtcctgataaagcagcatatcgatttttgcatcangataattctgcttgagcgtactgatgacaggagtagttaataacatatccccatgatatcnncnnnncnngtactagtcgacgcgtggcca

SEQ ID NO: 72 - DNA, 350 bp, Artificial
  partial gene with transposon insert
cgnccggatccgggatcatatgacaagatgtgtatccccttaacttaatgatttttaccaaaatcattaggggattcatcagcctggtgcctggaatgccgaacctggtatttttgctgttcactgccggattgctcgggctggcctggtggatacgcggacgcgaacaaaaagcgcctgccgaacccaaaccggtaaaaatggcagagaataataccgttgtcgaagcgacgtggaacgatgtacaactggaagattctctgggaatggaagtgggttatggactgatcccgatggtcgatttccagcaggatggtgagttgttgggccgtatacgcagtatccgcaag

SEQ ID NO: 73 - DNA, 380 bp, Artificial
  partial gene with transposon insert
acgtnccggatccgggatcatatgacaagatgtgtatccaccttaacttaatgatttttaccaaaatcattaggggattcatcagcctggtgcctggaatgcccgaacctggtatttttgctgttcactgccggattgctcgggctggcctggtggatacgcggacgcgaacaaaaagcgcctgccgaacccaaaccggtaaaaatggcagagaataataccgttgtcgaagcgacgtggaacgatgtacaactggaagattctctgggaatggaagtgggttatcgactgatcccgatggtcgatttccagcaggatggtgagttgttgggccgtatacgcagtatccgcaagaaatttgcccaggagatgggatatcn

SEQ ID NO: 74 - DNA, 287 bp, Artificial
  partial gene with transposon insert
ntnnnaaaacacgccggacatccggatccgggatcatatgacaagatgtgtatccaccttaacttaatgatttttaccaaaatcattaggggattcatcagcgctttgcccgccattttcgtatggggctgttcaacctgctgcgtcgtagcccggatatatccnccnggngcgtactagtcgacgcgtggccaannnnnnnncnncnnctagcnntaaaanngncatnanccncncncacaancacnnangnanncnttncnaaacnancgtannatanccccnnc

SEQ ID NO: 75 - DNA, 152 bp, Artificial
  partial gene with transposon insert
nnaaagcccgccggacatcccggatcgggatcatatgacaagatgtgtatccaccttaacttaatgatttttaccaaaatcattaggggattcatcagcgcaaagcctgctttganatccgtaaagngcggactaancnacncgnggncnaa

SEQ ID NO: 76 - DNA, 450 bp, Artificial
  partial gene with transposon insert
gngaaaaaaacgnaccggatccgggatcatatgacaagatgtgtatccccttaacttaatgatttttaccaaaatcattaggggattcatcagggttaaatccattaactcgcgcatcatttattattaccgaagatctcactcccacaattagccttgaagattattgtgccgattgggcagtcaacccgcctgatatccngnncnnnngtactagtcgacgcgtggccatanantnagctcntnctacnnccnnantcctatccacccgtggctncagnnancattcngnnnacaccanttacnnnccagnccncntcccccngnnctcnnctactcannacttcannanannatgncnttcnnnngcnnntcgntcncccacnacnncntttnttncnncctctnncnanctcnnccntnncnctntnattccncttttaccctnan

SEQ ID NO: 77 - DNA, 400 bp, Artificial
  partial gene with transposon insert
gnngaacgncggatccgggatatatgacaagatgtgtatccaccttaacttaatgatttttaccaaaatcattaggggattcatcagcactttgcattgattgcagtatggacatcagatccatcggcgtagcgcccagcgcattgagcgcgcgcaccacgttattgaggctggcgctggaacgtacgctttgcagcgaaccgccgctctggcgtaaatcgatctgcgtttgtggagtaaccacagtctgtccaccaccaaacggtgtatctggctggctgacattggcctgacgattaactgttactgagagattcccctgcgctaccgcgcagctgtcgagggtcacttcgcgattcatcaccaccgaaccggtgcgcgagttaatcactactttagc

SEQ ID NO: 78 - DNA, 450 bp, Artificial
  partial gene with transposon insert
tngnaaaaacgtnccggatccgggatcatatgacaagatgtgtatccaccttaacttaatgatttttaccaaaatcattaggggattcatcagggtcaggtctggatttaagttccatccttgatagcctcaccgccgcgcaaaaagcgacgctnacccccatttcaaatcagcaatcgtcgtttaccgctaaacttagcgcctacggtacgctgaaaagcgcgctgacgactttccagaccgccaatactgcattgtctaaagccgatcttttttccgccaccagcaccaccagcagcaccaccgcgttcagtgccaccactgcgggtaacgccatcgccgggaaatacaccatcagcgtcacccatctggcgcaggcgcaaaccctgaccacgcgcaccaccagagacgatacgaaaacggcgatcgccaccagcgacagtaaactca

SEQ ID NO: 79 - DNA, 397 bp, Artificial
  partial gene with transposon insert
nnannaacaatacgtcccaagnananntgacnagangtgtatccaccttaacttaatgatttttaccaaaatcattaggggattcatcngggnccggtctggatttaagtttccatccttgatagcctcaccgccgcgcaaaaagcgacgctaaccnccatttcanatcagcaatcgtcgtttaccgctaaacttagcgcctacggtacgctgaaaagcgcgctgacgactttccagaccnccaatactgcattgtctaaagccgatcttttttccgccaccancaccacctgcagcaccacngcgttcantgccnccnctgcgggnnacnccatcgccnggaaataccccattaccgntcanccatctggcanatgcngcnaacccttgaacacgc

SEQ ID NO: 80 - DNA, 331 bp, Artificial
  partial gene with transposon insert
aaaacgtccggatccgggatcatatgacaagatgtgtatccaccttaacttaatgatttttaccaaaatcattaggggattcatcagcgctcaatctgcgcgcccagcgtcaggaagtgctggcagcaaacatcgccaatgccgatacccctggttatcaggcgcgcgatatcgattttgccagtgaacttaaaaaagtcatgcaacgtggacgggatgcaaccagtgtggttgcactgacgatgacctcaacgcaacacattccggcgcaggcgctgacgcctcctaccgcagaactgcaataccgtattccggaccagccttcgcttga

SEQ ID NO: 81 - DNA, 276 bp, Artificial
  partial gene witht transposon insert
ataaaacggntaccggatccgggatcatatgacagatgtgtatccaccttaacttaatgatttttaccaaaatcattaggggattcatgagggcgctgtttccacagcccttttaacgtttcaggcattaatatccagcacagtgnccctngnnnncncnnnnncntccactgattcaactgcagcttccagctaatatcaatatcttcggtgatcatatnagtccacncggnnctagtcgacgcgtggccannantnnnncnttntttntnnctn

SEQ ID NO: 82 - DNA, 264 bp, Artificial
  partial gene with transposon insert
tttaaaaaccggttaccggatccgggatcatatgacaagatgtgtatccaccttaacttaatgatttttaccaaaatcattaggggattcatcagtgccaggcgtcttttccggccattgtcgcagcactgtaacgcgtaaaatagtgctttctcttactcttctggctggaccatgagacttctgattctgactctttcattaatatccnnnncnnnngtactagtcgacgcgtggccanannantnntatntttnannacnn

SEQ ID NO: 83 - DNA, 242 bp, Artificial
  partial gene with transposon insert
ttgaaaaccgctccggatccnggatcatatgacaagatgtgtatccaccttaacttaatgatttttaccaaaatcattaggggattcatcagcgttaaattggcatcgtcatcgcggcagcaagttgattattacgtaatgcctgcacgtaattctgtgggatatccnncccnnncgtactagtcgacgcgtggccatntnacntncngcaatncnttctgacacttcnnnttnctntnnat

SEQ ID NO: 84 - DNA, 338 bp, Artificial
  partial gene with transposon insert
gcnngcggtaaacgnccggatccgggatcattgacaagatgtgtatccaccttaacttaatgatttttaccaaaatcattagggggattcatcagagttaagtgtgacagtgggagttaagtatgccagaggtacaaacagatcatccagagacggcggagttaagcaaaccacancttncgnatggncgatttcaacttattaacncgtttcgatgccntgntgnaggagccnancattnttcncagcccgctcatcgccctgggaaccttcnccccccctncnttcctntgctngccttggggcccccnncacncaacgnagacngggccnatccc

SEQ ID NO: 85 - DNA, 259 bp, Artificial
  partial gene with transposon insert
aaccgtaccggatccgggatcattgacaagatgtgtatccaccttaacttaatgatttttaccaaaatcattagggggattcatcagtgcnacgncnctntnannnccatngatntngcactgtaacgcgctaaataacgcttgtntcttactcttctggctggaccatgagacttctgattctgactctttcattaatatccccccncnccgtactagtcgacgcgtgncnatattatgnnnccnnnnnctantnnnc

SEQ ID NO: 86 - DNA, 250 bp, Artificial
  partial gene with transposon insert
cnnccggatccgggatcatatgacaagatgtgtatccaccttaacttaatgatttttaccaaaatcattaggggattcatcagaggcaagtgtcaataatctgacgagcaagtttatttcgttccattagctacctctctctgattcaaaacagggcaataatgttgttcctttcacactattgaattagccgtttaattacccaccatcttcttcctgattaacaagaaagaaattcacaagcttatat

SEQ ID NO: 87 - DNA, 390 bp, Artificial
  partial gene with transposon insert
aacgnccgattccggatcatatgacaagatgtgtatccaccttaacttaatgatttttaccaaaatcattaggggattcatcagccagatgcgttcctgaccctcaagtgagacttcatgacgctggttactgtcggaaaaatcagtgaaagcccaggagacaccgtacttacgccgctcattttgataccagcgaacataagcctgagcactgttgcctgtaacaccatttttcattgcccgtaatggaacgcggtgagagaggcgttccagttgcgaaccaatacgccagttatcattaaaatcataccagccagacaggcgcgcgccgggtttatgctcatgattgaaaacgcgttcagcgtactctgcctcgagccagatattacg

SEQ ID NO: 88 - DNA, 314 bp, Artificial
  partial gene with transposon insert
aacgnnccggatccgggatcatatgacaagatgtgtatccaccttaacttaatgatttttaccaaaatcattaggggattcatcagtgcccagggttttcaccttgcaatggccgggtataaacaggcaggaaattganancantgagncatnnncnacganaccaattnctggcntngccgggcattatctttttaattctctttcccatcattctttcgctatggattgccttcctttgggcaaaatcagaagtgaataatcagctccgaacctttgctcaactggcactggataaatccgagctggtcatt

SEQ ID NO: 89 - DNA, 432 bp, Artificial
  partial gene with transposon insert
cncgccggacatccggatccgggatcatatgacaagatgtgtatccaccttaacttaatgatttttaccaaaatcattaggggattcatcaggtgacagttagcgcgcccggataattttcactctccaggtggctgtaaaagagatccgcaagttcttcatcggataaatccggggcaatggtctccttgtgataaaagagctcggtcattattgactgtgcttttttcggctgatgatctttgagataagccgatgcaacccaatattgcccccagggcggaataatttgccccgtcttttttaatcgctgatagtgagaaataacgtctttataacgatcgcgagttaataacgcgccaagatgatcaacctgaatacgctggtactgggcagtgcggtctgggttatcgtgccacagaatttctaatg

SEQ ID NO: 90 - DNA, 209 bp, Artificial
  partial gene with transposon insert
aaacgaccggaccggatcatatgacaagatgtgtatccaccttaacttaatgatttttaccaaaatcattaggggattcatcagtgccnggcgtntttnccggccattgncncagcactgnnacgcgtaaaatagngctttctcttactcttctggctggaccatgagacttctgattctgactctttcattaatatcccccccccccg

SEQ ID NO: 91 - DNA, 450 bp, Artificial
  partial gene with transposon insert
ngannatacgnccgatccgggatcatatgacaagatgtgtatccccttaacttaatgatttttaccaaaatcattaggggattcatcaggttgaaacatcttataaggggtggcaaaactcaccggggatgcaagcgaaacaggggagtcattgcttagatgatgacaggtaatggcgcggatatcgaatgttatgcaancaganaanancagnctgttcaaatggctgtgcgattctggatagcccgaaatagtcaacttcaggctatccagagagcggaattattccgccaaagtgcgtttttgctgttcgagatcgcgttcaatgccgtcacgaacatcctgggggattttcagcgcgtcacccagtgcattcaggtaactgcgttccataaaatggtcaatatcaatagccgcgnnactaatnnacncgnggccnagcgcctcttc

SEQ ID NO: 92 - DNA, 469 bp, Artificial
  partial gene with transposon insert
cgnccggatccgggatcatatgacaagatgtgtatccaccttaacttaatgatttttaccaaaatcattaggggattcatcagtgccgacggtcttttgagcgtgacggcgatggagaaatccaccggcgttgaggcgtctattcaggtcaaaccgtcttacggtctgaccgatagcgaaatcgcttcgatgatcaaagactcaatgagctatgccgagcaggacgtaaaagcccgaatgctggcagaacaaaaagtagaagcggcgcgtgtgctggaaagtctgcacggcgcgctggctgctgatgccgcgctgttaagcgccgcagaacgtcaggtcattgacgatgctgccgctcacctgagtgaagtggcgcanggcgatgatgttgacgccatcgaacaagcgattaaaaacgtagacaaacaaacccaggatatcncccnnnncngtactagtcgacgcgtgg

SEQ ID NO: 93 - DNA, 212 bp, Artificial
  partial gene with transposon insert
ngaaaaacgtccggatccgggatcatatgacaagatgtgtatccaccttaacttaatgatttttaccaaaatcattaggggattcatcagctccgcgccctggtttttaacattacccaccgcgcgcgatatcnnnnnnnnnngtactagtcgacgcgtgggccaantannnnnncnnnntnnnnnnntnnnnnncgnntnnngnnnnncnc

SEQ ID NO: 94 - DNA, 290 bp, Artificial
  partial gene with transposon insert
aaaaaccgttaccggatccgggatcatatgacaagatgtgtatccaccttaacttaatgatttttaccaaaatcattaggggattcatcagagttaagtgtgacagtggagttaagtatgccagaggtacaaacagatcatccagagacggcggagttaagcaaaccacagctacgcatggtcgatctcaacttattaaccgttttcgatgccgtgatgcaggagcaaaacattactcgtgccgctcatgttctgggaatatcccccccnncngtactagtcgacgcngn

SEQ ID NO: 95 - DNA, 269 bp, Artificial
  partial gene with transposon insert
aaaaaccgttaccggatccgggatcatatgacaagatgtgtatccaccttaacttaatgatttttaccaaaatcattaggggattcatcagagttaagtgtgacagtggagttaagtatgccagaggtacaaacagatcatccagagacggcggagttaagcaaaccacagctacgcatggtcgatctcaacttattaaccgttttcgatgccgtgatgcaggagcaaaacattactcgtgccgctcatgttctgggaatatccccccc

SEQ ID NO: 96 - DNA, 404 bp, Artificial
  partial gene with transposon insert
cncgccggacatccggatccgggatcatatgacaagatgtgtatccaccttaacttaatgatttttaccaaaatcattaggggattcatcaggtgacagttagcgcgcccggataattttcactctccaggtggctgtaaaagagatccgcaagttcttcatcggataaatccggggcaatggtctccttgtgataaaagagctcggtcattattgactgtgcttttttcggctgatgatctttgagataagccgatgcaacccaatattgcccccagggcggaataatttgccccgtcttttttaatcgctgatagtgagaaataacgtctttataacgatcgcgagttaataacgcgccaagatgatcaacctgaatacgctggtactgggcagtgcggtct

SEQ ID NO: 97 - DNA, 291 bp, Artificial
  partial gene with transposon insert
aaaaaccgttaccggatccgggatcatatgacaagatgtgtatccaccttaacttaatgatttttaccaaaatcattaggggattcatcaggagctggngcgttttctccccacggccagtggcgttcacnatagacccagaaatagacgccgncnacaatncacataatggacataaaaaacggccagaagaaaacgaacctnatcatcagttcaccgagtgaaagtacgctacgcataggggtatgcataacaccagacataatataaaaaacgatacgatgcgattaa

SEQ ID NO: 98 - DNA, 400 bp, Artificial
  partial gene with transposon insert
gagatacgnccggatccgggatcatatgacaagatgtgtatccaccttaacttaatgatttttaccaaaatcattaggggattcatcagtgctccaccctggcccggatgctggctttcatgtattcgatgttgatggccgttttgttcttgcgtggatgctgtttcaaggttcttaccttgccggggcgctcggcgatcagccagtccacatccacctcggccagctcctcgcgctgtggcgccccttggtagccggcatcggctgagacaaattgctcctctccatgcagcagattacccagctgattgaggtcatgctcgttggccgcggtggtgaccaggctgtgggtcaggccactcttggcatcgacaccaatgtgggccttcatgccaaagtg

SEQ ID NO: 99 - DNA, 434 bp, Artificial
  partial gene with transposon insert
cgnccgatccgggatcatatgacaagatgtgtatccaccttaacttaatgatttttaccaaaatcattaggggattcatcaggatgaattacgggcgatgacagagtcattacctgaaaatgcatctacgcaacaatatcccacnnantngtactagncgacgcgtggccatcaacttgctgccgcgattgacgatgccaatttaacgccagatattcgcgctgatattnctanncgacgngcnggnngacgcgtggccaangcnnnncntnnnctnnnaannntgnncngnncnctggctgntgtccnnnctgnnancgccncanaacntcntgncntnnnnnangctgncgtcccttanngaagnggccnnggnnaatnatgtnnacnccntnnccaancgnttnnnnactnnacnancnacccnngatntc

SEQ ID NO: 100 - DNA, 250 bp, Artificial
  partial gene with transposon insert
cacggatccgggatatatgacaagatgtgtatccaccttaacttaatgatttttaccaaaatcattaggggattcatcagcgttaaattggcatcgtcaatcgcggcagcaagttgattattacgtaatgcctgcacgtattctgtgggatatccnncccncccgtactagtcgacgcgtggccatgnnatnnnccgnnattcatnctgatgacnccccgncagtttatanatatnnnnnnnnnngtnct

SEQ ID NO: 101 - DNA, 458 bp, Artificial
  partial gene with transposon insert
aacgcaccggatccgggatcattgacaagatgtgtatccaccttaacttaatgatttttaccaaaatcattaggggattcatcagactgacgcgcccgcaaaaagggcggattgtcctcaatgggcgggtactaaatgatgccgaaaaaggtatctgccttnccgtactagtcgacgcgtggccacnnnttcattcnccnctanctcnnaagtnnnccnactccgngnncnaccnnccnnnccccntngcagncntgtncncnnacnncggcnacccnnngannncgnctccncccgcccctntcnncacctnnnnanggcntaccngcccctcnngctcngttaccttnntnnntnnccgnccccnctcananncnccntnacnngncnnncnatncntcgcnnnnnnagtanncccncntccccccacncncnnnccgtnttnncncttnaganct

SEQ ID NO: 102 - DNA, 455 bp, Artificial
  partial gene with transposon insert
aacgnaccggatccgggatcatatgacaagatgtgtatccaccttaacttaatgatttttaccaaaatcattaggggattcatcagactgacgcgcccgcaaaaagggcggattgtcctcaatgggcgggtactaaatgatgccgaaaanggtntcttgcctgncccgtactantcgacgcgtggccaacnaacctncttcntannntcgcnnttcccccttcngcncntcnnccactcccngctgctcctncnnccttccncgcnccntaccntcgtnnccttantccacctgcnnctatcccncggcccgncctcccgnccccccnctncaantngttcangnacngnccnccctcgcccnagcgctnccngngccagnnnctnttcatntccctcccngatccantcncnnccnttncnctctnacnnnccngtcnctnnccccctnnttan

SEQ ID NO: 103 - DNA, 300 bp, Artificial
  partial gene with transposon insert
cncgaccgggatatatgacaagatgtgtatccaccttaacttaatgatttttaccaaaatcattaggggattcatcagggtttcgctgtccttttgtgctttgttatgcattttctgcgcagattcactttgcgaggcaatatccatggcatcaaaanaatggcaactacggcacgttgcagagtcagtggcttttaattctttccatactgtttcggccatttcctggcgatgagcttcgaacttatcgtcactgtctattttgccgctaacaaattcatgataaatatctttagatgc

SEQ ID NO: 104 - DNA, 527 bp, Artificial
  partial gene with transposon insert
aaacnnccggatccgggatcatatgacaagatgtgtatccaccttaacttaatgatttttaccaaaatcattaggggattcatcanggttttgacccatgacagataaacgcaaagatggctcatgcanattgctgtattgctctttttgcggcaaaagccnncatgaagtgcgccagctgannagccggnccatccctntatatcnnccnnccctgtacctngncggncncgtggncnnnctcctntcnttnncntctccccnntctnnnncccctctnncgcggncccnctganncgcctcncttcttntacctccncgngcctctaccnctcgnncctcnccccccacctccttattccncctccntcntntccntctccacctntcctttccnaccgcnncatnnnacgtctcnttccctnnnnccacnnntnatccttcngcncccctcngngcgaancntcnctnnnctancggcncgntgnncntgcnncncantnancncncnctnattgagtgcgngt

SEQ ID NO: 105 - DNA, 233 bp, Artificial
  partial gene with transposon insert
cncgatccgggatattgacaagatgtgtatccaccttaacttaatgatttttaccaaaatcattaggggattcatcaggtgacgacatccgtgccataatgtttttgccaggaggcaccaacacctgcgctgaatatttgctcccagctattttcatagcttcgccataacaaatggcttgcctcaaatgccggaacaatatcnnncnnnnnngtactagtcgacgcgtgcca

SEQ ID NO: 106 - DNA, 492 bp, Artificial
  partial gene with transposon insert
aaacccaccggatccgggatcatatgacaagatgtgtatccaccttaacttaatgatttttaccaaaatcattaggggattcatcagagttttatctgcgtctggcgcaacgcattatgcatctgttcagtacgcgtacctcttccggcattttgtatgaagtggatgctcgactgcgtccgtccggggcggcgggaatgctggtgacatccgcagaagcatttgccgattatcagaaaaacgaggcctggacgtgggaacatcaggcgctggtgcgtgcgcgtgtagtgtacggcgatccgcagctcaccgcgcactttgacgcagtgcgtcgcgagattatgacgctgccgcgtgaaggtaaaactctgcaaacggaagtgcgggaaatgcgcgagaaaatgcgcgctcatctcggcaataaacatcgcgatcgctttgatatcaaagctgatgaagggggaattaccgatatcgaatttattacccaat

SEQ ID NO: 107 - DNA, 402 bp, Artificial
  partial gene with transposon insert
gncnnctaaaaacnttaccggatccgggatcatatgacaagatgtgtatccaccttaacttaatgatttttaccaaaatcattaggggattcatcanggccgnnanggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacgg

SEQ ID NO: 108 - DNA, 483 bp, Artificial
  partial gene with transposon insert
cgnccggatccgggatcatatgacaagatgtgtatccaccttaacttaatgatttttaccaaaatcattaggggattcatcagcgcatctggtcttcaccatatttgattgtcgatttcctacccagtctgtattacgaacaaaatacagaacacgataccccatactacaaccctataaaaacgttcgatatngttccggcntgtgctgcncgncnngtggnctggcgaagctatgaaaatagctgggagcaaatattcagcgcaggtgttggtgcctcctggcnaaaacattatggcacggatgtcgtcacccaactcggctacgggcaacgcattagttggaatgacgtgattgatgctggcgcaacgctacgctgggaaaaacgaccttatgacggtgacagagaacacaacttatacgttgaattcgatatgacattcagattttaaggataaatatgttacgtaatggaaataaata

SEQ ID NO: 109 - DNA, 455 bp, Artificial
  partial gene with transposon insert
acgncggatccgggatcatatgacaagatgtgtatccaccttaacttaatgatttttaccaaaatcattaggggattcatcagcaccgtacttacgccgctcattttgataccancgaacataagcctgagcactgttgcctgtaacaccatttttcattgcccgtaatggaacgcggtgagagaggcgttccagttgcgaaccaatacgccagttatcattaaaatcatatcngccngnccngtactagtcgacgcgtggccanaaattgaaaactggtttgccanaattntctngatcncctaaaaagctatnactggacncgntatnatggntntgnnttatctggnanggggnnncanaaaaatncgntgccaatggntnatncaattgnccatnaaattaaaaacatcccttangntnaaagacaaatnnattttntaattcangggcna

SEQ ID NO: 110 - DNA, 468 bp, Artificial
  partial gene with transposon insert
nngnaaaccagccggacntcccggatccgggatcatatgacnagatgtgtatccaccttaacttaatgatttttaccaaaatcattangggattcatcagncagatgcgttcctgaccctcaagngagacttcatgacgctggttactgtcggaaaaatcagtgaaagcccagganacaccgtacttacgccgctcattttgataccagcgaacataagcctgagcactgttgcctgtaacaccatttttcattgcccgtaatggaacgcggtgagagaggcgttccagttgcgaaccaatacgccagntatcattaaaatcatatcnnccngaagaggactagtcnacgcgtggccannacanccncactnntnaacntgnggctacnantntaccngcccannagnnttacntnanttncgcncncctnccantcncncccnnangtnnncnnaanctnnannctn

SEQ ID NO: 111 - DNA, 313 bp, Artificial
  partial gene with transposon insert
nnaanatacggttccggntcgggatcatatgacaagatgtgtatccaccttaacttaatgatttttaccaaaatcattaggggattcatcagatggaagcattaaccgcggcctccgtggcggcgctgaccatttatgacatgtgcaaagcggtgcaaaaagatatcncnnnccnnngtactagtcgacgcgtggccaaanatcngggntctcnnnntgctngctncnaatcnantgnacccnctnaacccnttcnagctaaacatntnnatntgnaacnnataaacncaggacgncactatngngtnnacnt

SEQ ID NO: 112 - DNA, 398 bp, Artificial
  partial gene with transposon insert
cgnnganacgnccgaatccggatantanacaagatgtgtatccccttaacttaatgatttttaccaaaatcattaggggattcatcagggctttagcgggagtctgagcgaaattcgacaaaacccggagcaatttaaacagtgggcccgctttaaaagtcgtgcgttaactgacttcactttagaacttagtgcgcgcgtaaaagccattcgcggtccacatattaaaactgcacgaaatatttttgcacttccggtaatacaacctgaaagtgaagcctggtttgcacagaattatgctgatttcctaaaaagctatgactggaccgctattatggctatgccttatctggaaggtgtcgcagaaaaatcggctgaccaatggttaatacaattga

SEQ ID NO: 113 - DNA, 300 bp, Artificial
  partial gene with transposon insert
nacggatccgggatatatgacaagatgtgtatccaccttaacttaatgatttttaccaaaatcattaggggattcatcagtatctggtgtgcattgatggcgatgcgttattagaccgcgatgcggcggcatatattgtggaaccgatgttgtacaacccgcgtgtgggtgccgtaaccggtaatcctcgtattcgaacacgttctaccctggtgggtaaaattcaggttggcgagtattcctcaattattggtttgatcaagcgaacccagcgtatctatggaaacgtatttaccgttt

SEQ ID NO: 114 - DNA, 300 bp, Artificial
  partial gene with transposon insert
ncgatccgggatcattgacaagatgtgtatccaccttaacttaatgatttttaccaaaatcattaggggattcatcagtatctggtgtgcattgatggcgatgcgttattagaccgcgatgcggcggcatatattgtggaaccgatgttgtacaacccgcgtgtgggtgccgtaaccggtaatcctcgtattcgaacacgttctaccctggtgggtaaaattcaggttggcgagtattcctcaattattggtttgatcaagcgaacccagcgtatctatggaaacgtatttaccgtttcc

SEQ ID NO: 115 - DNA, 453 bp, Artificial
  partial gene with transposon insert
aaacntaccggatccgggatcatatgacaagatgtgtatccaccttaacttaatgatttttaccaaaatcattaggggattcatcagcggcgagcaggcgatttccaccgaggcttatatcccccccnnccgtactagtcgacgcgtggccaantatcnngatnnccccgcctgtcanncnanccccccctgcgncccctcntccnactcnancntagaccngccnctnntnnnncanctcncggcgcntgtnccaacctgcnccnccccgtcctgccnacnctctccacgncncnnccnnttncccngccctgccnnctctcccccccnccnccacnctcngnccctcagncncngtcccgctcggtncnccnacncnncncnnnnagctctccngttacctcaccgatgntcncccccctcgngngcncaccccnnagcagccncntnnnc

SEQ ID NO: 116 - DNA, 268 bp, Artificial
  partial gene with transposon insert
aacggnccggatccgggatcatatgacaagatgtgtatccaccttaacttaatgatttttaccaaaatcattaggggattcatcagagntaagcgtgacagtgntantnnnctacngccanaggtacaaacaanttcatncatnngccgcngaagatttccttntnnnagcnnncagccnngnntnttnnncantntcctaacccntnncncntggtngtnannncangttcatnnnncattnantgnntntntcgantncgcntcnc

SEQ ID NO: 117 - DNA, 263 bp, Artificial
  partial gene with transposon insert
gnaaagcccgccggacatcccggatccgggatcatatgacaagatgtgtatccaccttaacttaatgatttttaccaaaatcattaggggattcatcagagccggaagtnaacgcggngctggagaagatgaaaaccttctcanaagcgattatttccggtgagtggaaaggnnataccggcnaagcaatcactgacgtagtgaatatcncncnccccngtactagtcgacgcgtggccacannnnnnnnnnnnncnnnnnnc

SEQ ID NO: 118 - DNA, 390 bp, Artificial
  partial gene with transposon insert
acgaccggatccgggatcatatgacaagatgtgtatccaccttaacttaatgatttttaccaaaatcattaggggattcatcagtgctcgccgccttctttggcactgcggaaggtaaacagcagcngtttttctggcatggactcacggagaatttttgctgccgccatgacagactccacattggagnggtangcatnctggnccacacnccattccanantatcnnctttnnctgcnactantnannnctcgnantnttncntcncnnnncncncanttcctcnnccttnnacnnncgngnnnnttgttgaannnttnnnacancncannttcnccccncntctnntanatnngncccnngcctnnnagtnntantncnntttnntc

SEQ ID NO: 119 - DNA, 392 bp, Artificial
  partial gene with transposon insert
cgtaccggatccgggatcatatgacaagatgtgtatccaccttaacttaatgatttttaccaaaatcattaggggattcatcangggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactt

SEQ ID NO: 120 - DNA, 451 bp, Artificial
  partial gene with transposon insert
cgnccggatccgggatcatatgacaagatgtgtatccaccttaacttaatgatttttaccaaaatcattaggggattcatcagggctttagcgggagtctgagcgaaattcgacaaaacccggagcaatttaaacagtgggcccgctttaaaagtcgtgcgttaactgacttcactttagaacttagtgcgcgcgtaaaagccattcgcggtccacatattaaaactgcacgaaatatttttgcacttccggtaatacaacctgaaagtgaagcctggtttgcacagaattatgctgatttcctaaaaagctatgactggaccgctattatggctatgccttatctggaaggtgtcgcagaaaaatcggctgaccaatggttaatacaattgaccaatcaaattaaaaacatccctcaggctaaagacaaatctattttagaattacaggc

SEQ ID NO: 121 - DNA, 300 bp, Artificial
  partial gene with transposon insert
ncaacgtnccggatccgggatcatatgacaagatgtgtatccccttaacttaatgatttttaccaaaatcattaggggattcatcaggtttatgctgacttaattgctccagcagcatttcgtgggtggattcgaagcagcgaagatgaatgggctcgccttctaccactgcggcgttaagtacgctactgaccaggcgtttggaaagtgcatcagccacgccaacgtcaaacaataaattggattctttgcgatagttcacaatatccnncnnnnccgtactagtcgacgcgtggccan

SEQ ID NO: 122 - DNA, 393 bp, Artificial
  partial gene with transposon insert
aaacncaccggatccgggatcatatgacaagatgtgtatccaccttaacttaatgatttttaccaaaatcattaggggattcatcagagttaagtgtgacaagtggagttaagtatgccanaggtacaaacagatcatncagagacggnggagttaagcaaaccacagctacagcatggtcgatctcaacttattaaccgttttcgatgccgtgatgcaggancaaaacattactcgtgccgctcatgttctgggaatatcncnnnngcnngnactagttcgacgcgtggccncatannatgtntcnnnttcnnnnctcttcnnttgcttcccttncccctcttccngccccccannncgtctnntntnnatcnngnncttnncntacgactn

SEQ ID NO: 123 - DNA, 331 bp, Artificial
  partial gene with transposon insert
aaacccntnggatccngcatcatatgacaagangttgtatccaccttaacttaatgattnttaccaaantcattaggggattcatcanagttaagtgtgacagtggagttaagtatgccagaggtacaaacagatcatccagagacggtggagttaagcaaaccacagctacngcatggtcgatctcaacttnttaaccgttntcgatgccgtgatgcaggagcaaaacattactncgtgccgntcatgttctgggaatatcccnnggnnacgtnctagtcnangcgtggccaanngntnnggnnnctnantcacagnanctttannngtn

SEQ ID NO: 124 - DNA, 298 bp, Artificial
  partial gene with transposon insert
agcncgccggacntcccggatcgggatcatatgacaagatgtgtatccaccttaacttaatgatttttaccaaaatcattaggggattcatcagtcggtagttaagcagaaattaatatcgcttactttaaccaccgcagcacaattagctaattttacggatgcagaactcacgctggcgggacgtttttattgcgtcagggttgacatccgtttttgtatccagtaactctaaaagcatatcgcccccnccngtactagtcgacgcgtggccanaacncgnnnntccantntnncc

SEQ ID NO: 125 - DNA, 411 bp, Artificial
  partial gene with transposon insert
gnaangnaaaacncgccggacatcccggatccgggatcatatgacaagatgtgtatccaccttaacttaatgatttttaccaaaatcattaggggattcatcagnacngcnnattgnncccacggnggantaantngccccngnttgnnntctcgctgntaanganaaataacgtctttataacgancgcgagtnaataacgcgccaagatgatcaacctgaatacnctggtactgggcagtgcggtctgggttatcgtgccacagaatttctaatgcagcgtattgggcgagggcgcgatcggcaatggcataacgntcactttcactgcgcgtaggcataaacgacagtctgaccagttcggcatgaatatcagnnnttgntgnactagncnacgcgtggncaaaacat

SEQ ID NO: 126 - DNA, 388 bp, Artificial
  partial gene with transposon insert
cgnccggatccgggatcatatgacaagatgtgtatccaccttaacttaatgatttttaccaaaatcattaggggattcatcagagcttaataccggcatccacgcatagatgtttacacctgagcgggtacgtaattgccaggcaacccgactaaaaatatctgctttcattggnctnanacaancgngganccagacctctttgaccagcccatcaccatcgggatcagcaaatgcctgcaaatacacggttgatatttgcatatctttcccccncgtactagtcgacgcgtggccacattacttntantntannantggatccnantnanncggntntanctngccttgnangggngnnannattatcnnctgccnncgacnaant

SEQ ID NO: 127 - DNA, 410 bp, Artificial
  partial gene with transposon insert
tttnngaaacacgccgnacatcccggatccgggatcatatgacaagatgtgtatccaccttaacttaatgatttttaccaaaaatcattaggggattcatcagtggattatgcggttgataaaaaagcgatcgatacagctacgcgtcagacactgctcggcaatagcctggtcgttgtagcaccgaaagccagcgtgcagaaagatttcaccatcgacagcaaaaccaactggacttcactgctgaatggcggtcgcctggcggttggcgatccggaacatgttcccgctggcatttatgcaaaagaagcactgcnaaaactgggcgcatgggatacnctcnctccgaaaatggncccncntaagntgntcctaggggnncnnncgcnggtcaccacntaatcgaaccn

SEQ ID NO: 128 - DNA, 305 bp, Artificial
  partial gene with transposon insert
gcaaaaacganncggccaaggatcatatgacaagatgtgtatccaccttaacttaatgatttttaccaaaaatcattaggggattcatcaggaaagggcagcnttancgccggcatgaataccgatgacattctcaccttcgccattaacaaaaatcancgccacacctgttgattcgcctttgatctcgctgaccggagtaanatccccntnnnngnactagtcnacgcgtggccatnaanttcnnncgaccntangacccnantcctgnnnttaanncncgnntnccntanttgcnccannnn

SEQ ID NO: 129 - DNA, 450 bp, Artificial
  partial gene with transposon insert
cacanccgnacatcngcgntatnnctactnanaatgncgtgaantattcncnactgcntnacacttacaccgnntctcatnccggtangcaccacanaaannttgaatatgaggcatgaatggtngttggangccgggtaacagccntcattatgggngttggcctcaacacgattttcctccntttaaaaaactcacgccgntaccctgtaacctcgcaccatactggcagggcagcngacngattcgcctgcgcatgaaatgnanaaacnntggggatncaccagggngtaaatctngccagcgatnnctgnnttnntcttntganangatncngaaaangggggntgcncncgtntnccggnaaacatcnntaataaaactggggccaaagtaaaacgtcnggatatccaatccannngtactantcgacgcgtggccannactgnc

SEQ ID NO: 130 - DNA, 291 bp, Artificial
  partial gene with transposon insert
nnacgctncggnncgggatcatatgacaagatgtgtatccaccttaacttaatgatttttaccaaaatcattaggggattcatcanagttaagtgtgacagtggagttaagtatgccagaggtacaaacagatcatccagagacggcggagttaagcaaaccacagtctacgcatggtcgatctcaactattaaccgttttcgatgccgtgatgcaggagcaaaacattactcgtgccgctcatgttctgggaatatcnccccnctnngtactagtcgacgcgtggccann

SEQ ID NO: 131 - DNA, 203 bp, Artificial
  partial gene with transposon insert
atncncgaccgggatcatatgacaagatgtgtatccaccttaacttaatgatttttaccaaaatcattaggggattcatcanngtccagtaattcaattanaggaatctatgcgnggganaaacggntggcngctncncgctaangcnaanaantaanccncctnnnnctangtatnnnnggncnntnnnannncngntttct

SEQ ID NO: 132 - DNA, 327 bp, Artificial
  partial gene with transposon insert
anncnncgaccnggntcatatgacaagatgtgtatccaccttaacttaatgatttttaccaaaatcattaggggattcatcagtcnggccgancnggcnatttccaccgaggcttatntccncnnntntcgnnctagncgnngcntggccangnttncgnccnncntnacncnctccatnantntnnccnctntcncnnantacngtgcccgngnatncccntcctccgtcggcccccncnngcttagcngcnnantggccnnnnnncaanttangacgatcngccngcccnntnaacngngttngccatntncntncnctnttatn

SEQ ID NO: 133 - DNA, 484 bp, Artificial
  partial gene with transposon insert
cgnccggatccgggatcatatgacaagatgtgtatccaccttaacttaatgatttttaccaaaatcattaggggattcatcagaggcaagtccgtatgcaaatctcctcgggcaaaaagcgcccggacttctgcttcaaaacctgaagccaacatctgatgaaaacgctgctcaatgcgttgatggagcagttcacggctcgccggggcgatggcgaactgatgcacctgatacggtagagcgtctcctgacgtttgcgtcagttccgttaaagttttacccgaaatgaaaaaaacttccagtgcccgggaaagcctttgtggatcatttggatgaatccttgctgccgcaaccggatctacctcctgaagttgacgatgcaatgactcccaaccttgctctgccgcctgttgctcaattctggcccgtacttccgggtctgccgacggtagcggcgacaacccttccagcaatgccttgaaat

SEQ ID NO: 134 - DNA, 300 bp, Artificial
  partial gene with transposon insert
gncggatccggtatatatgacaagatgtgtatccaccttaacttaatgatttttaccaaaatcattaggggattcatcaggatggggcatcaaccagatagcttcattgcttaagaaaagtaataaaactatcancgcccaaaaaaacagtgcgatgcgtcgactggcaattcacagcaacgctgaaatgtatgcatggataaatagcgcgcagggtgcaagagaacttaacttgccttctgtttatggagatgccgcagaatggaacacagccgaattaagaagagaaatgtcgcactc

SEQ ID NO: 135 - DNA, 388 bp, Artificial
  partial gene with transposon insert
nnnaaagcacgccggacatccgatccgggatcatatgacaagatgtgtatccaccttaacttaatgatttttaccaaaatcattaggggattcatcagacccaatattgcccccagggcggaataatttgccccgtcttttttaatcgctgatagtgagaaataacgtctttataacgatcgcgagttataacgcgccaagatgatcaacctgaatacgctggtactgggcagtgcggtctgggttatcgtgccacagaatttctaatgcagcgtattgggcgagggcgcgatcggcaatggcataacgttcactttcactgcgcgtaggcataaacgacagtctgaccagttcggcatgaatatcancgcgaatagtactagtcgac

SEQ ID NO: 136 - DNA, 345 bp, Artificial
  partial gene with transposon insert
nnnngaagcncgccggacatcccggatccgggatcatatgacaagatgtgtatccaccttaacttaatgatttttaccaaaatcattaggggattcatcagtaccacgcccctaatattgctattgtaataccaataatttgctaaggcctctgactttctggccgcaatcaatacgacctgtttcccctttaccgcggataaccattcattatcacggtttataaattcagataaagaatccgtactgagattgataaacacgaaatcttttctaattttctccaacgatagataaattaatctgtcgatatcccccnncnnngtactagtcgacgcgtggcca

SEQ ID NO: 137 - DNA, 452 bp, Artificial
  partial gene with transposon insert
nacgaccggatccgggatcatatgacaagatgtgtatccaccttaacttaatgatttttaccaaaatcattaggggattcatcagggccagaagccgacttcagttctattttttgctggcagtggcgaatgccgtcgtgttaattgtctgggcgctgtacaataagctgcgttttcaaaaacagcagcatcatgcagcctaccaatatacgccgcaagaatatgcagagagcttagcaatacctgatgagctctatcagcaactacaaaaaagccacaggatgagcgtacacttcaccagccaggggcaaataaaaatggttgtttcagaaaaagcgctagtccgggcataaacacccaaaacaaagcccggttcgcccgggctctgcaccgataacacacttaactgtaggcatgcagcgtacgttggcaaagtgccgaacgtacgcant

SEQ ID NO: 138 - DNA, 350 bp, Artificial
  partial gene with transposon insert
anacncgtccgggatcatatgacaagatgtgtatccaccttaacttaatgatttttaccaaaatcattangggattcatcagtgtggtgtccggtaaatatgaaaaaggcatnaaactgcnccacgagcccactgcnaangatgnggnaatctccgccccgcngcctctctggcnggtnnccgttctcaccttnnacaccnccccnngctcncntncnctcccnnacnncnnnttctctcngcancccacttnatctnccnncnccctcccnacgnntcccnccccncngnncnantgnnttggctnncgnccnnnanncncnctcntcctggcctcnctcnttacncnn

SEQ ID NO: 139 - DNA, 190 bp, Artificial
  partial gene with transposon insert
nncgatccgggatcattgacaagatgtgtatccaccttaacttaatgatttttaccaaaatcattaggggattcatcagcacttgttgccacgtggcaaaatattctcgatcgaccaactcatcgccaaattttacttgtttatccgctggcatatcnncccnnnncgtactagtcgacgcgtggccann

SEQ ID NO: 140 - DNA, 455 bp, Artificial
  partial gene with transposon insert
cgnccggatccgggatcatatgacaagatgtgtatccaccttaacttaatgatttttaccaaaatcattaggggattcatcagggcatcaaccagatagcttcattgcttaagaaaagtaataaaactatcagcgcccaaaaaaacagtgcgatgcgtcgactggcaattcacagcaacgctgaaatgtatgcatggataaatagcgcgcagggtgcaagagaacttaacttgccttctgtttatggagatgccgcagaatggaacacagccgaattaagaagagaaatgtcgcactcatagaaaaatgcgtcatgagtagtatcggtattgagagtttattcagaaagtttgcgggtaacccttataagctccatacctataccagtcaggagtcatttcaggatgccatgtcgcggatatcccnncnccncgtactagtcgacgcgtggccaa

SEQ ID NO: 141 - DNA, 224 bp, Artificial
  partial gene with transposon insert
aaaancncgccggacatcccggatccgggatcatatgacaaggatgtgtatccaccttaacttaatgatttttaccaaaatcattaggggattcatcagtattaatcgccaataatccctcattaaaagagtttgcccagcaaattattaccgtacaggaaaaatcaccacaacggataatgcatatcncncnnnnnngtactagtcgacgcgtggccacanan

(19) United States
(12) Patent Application Publication
(10) Pub. No.: US 2005/0032093 A1
(43) Pub. Date: Feb. 10, 2005

(54) Novel genes involved in the escherichia coli biofilm formation and uses thereof

(76) Inventors: Tony Romeo, Decatur, GA (US); Xin Wang, Atlanta, GA (US); Rebecca L. Desplas, Dallas, TX (US)

Correspondence Address: MERCHANT & GOULD PC, P.O. BOX 2903, MINNEAPOLIS, MN, 55402-0903, US

(21) Appl. No.: 10/826,967

(22) Filed: Apr. 16, 2004

Related U.S. Application Data
(60) Provisional application No. 60/464,062, filed on Apr. 17, 2003.

(51) Int. Cl. C12Q 1/68

(52) U.S. Cl. 435/6

(57) ABSTRACT

The present invention relates to biofilm genes, their polypeptides, and the use of such genes, polypeptides and control regions in determining compositions for use in the disruption of biofilms and the treatment of disease. The identified compositions regulate the expression of the biofilm genes or modulate the activity of their protein products.

### CROSS-REFERENCE TO RELATED APPLICATIONS

This application claims priority to and the benefit of the filing date of U.S. Provisional Patent Application entitled “NOVEL GENES INVOLVED IN THE Escherichia coli BIOFILM FORMATION AND USES THERE OF,” assigned Ser. No. 60/464,062, and filed Apr. 17, 2003, the text of which is hereby incorporated by reference.

### FIELD OF THE INVENTION

This invention relates to the identification of genes involved in biofilm formation, methods of identifying compounds that modulate the activity of biofilm formation and/or effectively regulate the level of expression of biofilm formation; and to compounds so identified.

### BACKGROUND OF THE INVENTION

Microorganisms commonly attach to living and nonliving surfaces, including those of indwelling medical devices, and form biofilms made up of extracellular polymers. In this state, microorganisms are highly resistant to antimicrobial treatment and are tenaciously bound to the surface. Biofilms represent a distinct physiological state, designed to provide a protected environment for survival under hostile conditions. Costerton, J. W. et al., Science, 284: 1318-1322 (1999). Many chronic infections that are difficult or impossible to eliminate with conventional antibiotic therapies are known to involve biofilms. A partial list of the infections that involve biofilms included: otitis media, prostatitis, vascular endocarditis, cystic fibrosis pneumonia, meliodosis, necrotizing faciitis, osteomyelitis, peridontitis, biliary tract infection, struvite kidney stone and host of nosocomial infections. Costerton, J. W., et al., Science, 284: 1318-1388 (1999).

As awareness of biofilm significance increases so does the need to understand the genetic mechanisms involved in its formation. Research using molecular techniques has been successful in gaining some insight to this process. Gene fusion experiments have been used to observe the changes in gene expression during biofilm development (Lamont, et. al., 1999). Expression patterns of a series of genes during biofilm fornnation have been studied by using DNA arrays (Stanley, et. al., 2003; Whiteley et. al., 2001). Libraries of bacterial mutants created by transposon mutagenesis have been analyzed for their effects on biofilm development (Kolter et. al., 1998). Many of these studies take advantage of a straightforward microtiter plate assay for biofilm development (Huber, et. al., 2002). Formation of biofilm in the wells of a polyinylchloride (PVC) microtiter plate is visualized by crystal violet staining. This technique allows for the examination of many independently generated biofilms at one time (Danese, et. al., 2001) As a result of research efforts, important aspects of biofilm development are being revealed. It is now known that signal molecules for cell-to-cell communication, or quorum sensing, are necessary for normal biofilm development in various species of bacteria (Kolter, et. al., 1998). While it is recognized that flagellum mediated motility enables the bacterium to reach potential surface sites in certain species, current studies also implicate flagella as playing a role in surface attachment (Pratt, et. al., 1998). Surfacing sensing is an adaptive strategy bacteria use in biofilm production.

It has been demonstrated that cells physically sense a surface through inhibition of flagellar rotation (Alavi, et. al., 2001). Therefore, one of the first stimuli sensed by bacteria as they form biofilm is mechanical in nature. Other surface components, including fimbriae and exopolysaccharides, have been shown to help bacteria counteract the repelling electrostatic forces between the surface and the bacterial envelope (Prigent-Combaret, et. al., 2000). Extracellular proteins or carbohydrates are known to be required for interaction with inert surfaces.

Many of these studies have taken place using the genetically well-characterized laboratory strain E. coli K12. Using random insertion mutagenesis, transduction and the previously described biofilm microtiter plate assay, strains were screened for defects in biofilm production as well as other phenotypic changes of interest (Genevaux, et. al, 1996; Genevaux, et. al., 1999; Pratt, et. al., 1998; Prigent-Combart, et. al., 1999). These methods have identified several important gene products involved in the biofilm development of this strain (Watnick, et. al., 2000). It has been determined that while motility is important to biofilm development in the early stages, it actually antagonizes mature biofilm formation. Type I pili have been reported to be key structures involved in stable cell-to-cell or cell-to-surface interactions needed for biofilm formation (Pratt, et. al, 1998). Other gene products have been shown to be involved in adhesion as well. DsbA, which catalyses the folding of extracytoplasmic proteins, is involved in the production of fimbriae (Genevaux, et. al., 1999). A mutation in this gene greatly affects adhesion. The Cpx-signaling pathway has been shown to respond to physical changes caused by adhesion of E. coli to surfaces (Otto, et. al., 2002). Evidence indicates that the surface induced response of Cpx activates a variety of genes involved in enhancing cell-surface interactions. A mutation in the cpxR gene dramatically lowers the number of attached cells. Once colonization occurs at the surface, major changes occur in the pattern of gene expression. Using transposon mutants generated by a MudX transposon carrying a promoterless lacZ gene, one study demonstrated that many of the genes in E. coli were affected at the level of transcription upon surface colonization (Prigent-Combaret, et. al., 1999). The proU operon (encoding a glycine betaine transport system), ompC (a porin encoding gene), wca locus (involved in colanic acid synthesis), pepT (encoding tripeptidase T) and nikA (involved in the nickel transport system) all have increased expression in attached bacteria. However, the expression of the flagellin gene, fliC, as well as a protein containing 92 amino acids (f92) are reduced in sessile bacteria. Some of these changes were also seen when the bacteria were grown in high salt concentrations, indicating that an increase in osmolarity may trigger increased expression of some biofilm-induced genes.

Recent studies have established that the global regulatory system Csr (carbon storage regulator) of E. coli regulates biofilm production and dispersal (Jackson et. al., 2002). The Csr system is a global regulatory process that influences gene expression post-transcriptionally (Romeo, 1998). The active element of this system is a RNA binding protein designated CsrA (Romeo, et. al., 1993). CsrA acts by binding to RNA transcripts, facilitating their decay. The Csr system has been shown to activate metabolic pathways needed for exponential growth while repressing the expression of stationary phase genes (Romeo, 1998). The effects of the Csr regulon are many including glycogen metabolism, gluconeogenesis and motility (Romeo, et. al., 1993; Wei, et. al., 2001). A mutation in the gene encoding CsrA resulted in a significant increase in biofilm formation (Jackson, et. al., 2002). A csrA mutant forms a biofilm in the absence of surface factors known to be important in biofilm development. The effects of the CsrA mutation are greater than other known global regulators involved in biofilm formation, RpoS and OmpR. CsrA mainly, though not exclusively, affects biofilm formation through its control of glycogen metabolism. Previous studies have identified CsrA as a post-transcriptional repressor of the glgCAP operon (Yang, et. al., 1996). Glycogen synthesis and catabolism are essential for optimal biofilm development. Furthermore, ectopic expression of glycogen synthase (encoded by glgA) and the catabolic enzyme glycogen phosphorylase (encoded by glgP) significantly increases biofilm formation. Since a csrA mutant overexpresses the glycogen biosynthetic genes (glgCA) as well as the gene for glycogen catabolism (glgP), an increase in glycogen metabolism occurs. In a csrA mutant, this increase in glycogen synthesis and its subsequent turn over improves biofilm formation (Jackson et. al., 2002). It has been proposed that glycogen may serve as a carbon and energy source for the synthesis of adhesions or other factors necessary in biofilm production during stationary phase.

Since motility and surface attachment abilities in E. coli are important in the initiation of biofilm production (Jackson et. al., 2002), many of the mutants generated in biofilm studies involve these processes (Pratt, et. al., 1998). To decrease the probability of creating mutations in genes already associated with biofilm production, a non-motile (ΔmotB) attachment deficit (ΔfimB-H) strain of E. coli K12 MG1655 was used as the parent strain in constructing a library of mini Tn10::cam transposon mutants. Such a strain forms little to no biofilm. When E. coli MG1655 is rendered non-motile by a motB deletion, biofilm formation is virtually eliminated. A mutation in motB, whose product is involved in the flagellum motor switch, renders the bacteria paralyzed although flagellum synthesis continues (Pratt, et. al., 1998). A type I pili deletion dramatically reduced biofilm formation as well (Jackson et. al., 2002). Deleting fimB-H creates a strain unable to produce type I pili (Pratt, et. al., 1998). To facilitate the isolation of biofilm down mutants, a csrA mutation was introduced into this strain. In a csrA mutant, these deletions decreased biofilm formation as compared to the wild type, however it was not eliminated (Jackson et. al., 2002). The csrA mutation would allow for the identification of genes whose interruption altered the biofilm phenotype even in the absence of motility and type I pili. The identification of those interrupted genes would result in the description of novel genes involved in the process of biofilm formation.

### BRIEF DESCRIPTION OF FIGURES

FIG. 1 is a schematic diagram of a primed polymerase chain reaction.

FIG. 2 is a schematic diagram of genes or operons for complementation studies.

FIG. 3 is a schematic diagram of vector pCR2.1 with insertion cloning site and resistance gene for Ampicillin.

FIGS. 4A and 4B are charts showing cell growth (total turbidity at A630 before staining) and biofilm formation A630 after crystal violet staining) absorbance readings for parent strains and representative mutant strains.

FIG. 5 lists sequences for mutants of the invention.

### SUMMARY OF THE INVENTION

Transposon mutagenesis using a mini Tn10::camR resistance marker yielded 800 random insertion mutants displaying altered biofilm phenotypes as compared to a non-motile, attachment deficient csrA mutant strain. Co-transduction with the resistance marker confirmed the linkages of 120 biofilm mutants (see the Sequences of Down Mutants and Up Mutants). Amplification of the insertion sites nucleotide sequencing and BLAST searches against E. coli K-12 genomic databases, successfully identified 118 of these sites. Many of the interrupted genes were not presently known to be associated with biofilm formation. A plasmid clone of the nhaAR operon complemented the corresponding mutation and increased biofilm formation by ˜10-fold. Transduction of four of the other interrupted genes into E. coli K-12 MG1655 produced altered biofilm phenotypes. Sixty-one different genes identified in this library of mutants influence biofilm formation.

Although the complete genome of E. coli K-12 was reported by Blattner (Science 277: 1453(1997) the report failed to suggest the involvement of these genes in biofilm formation. The invention also relates to the potential use of these genes or their products for modulating biofilm formation. In particular, the invention relates to use of these genes or their products as targets for developing antimicrobials, including antibiofilm products.

From 120 mutants of Escherichia coli K-12 strain produced by transposon mutagenesis, the involvement of sixty-one genes in Escherichia coli biofilm formation has been established.

Genes involved in the biosynthesis of flagella (flgB, flgE, flgH, flgI, flgK, flhA, flhB, flhD, fliA, fliD, fliF, fliG, fliI; fliL, fliM, flip, fliR, dnaK and mdoG), genes involved in lipopolysaccharide biosynthesis (rfaQ, rfaG, rfaP and wecB), gene affecting swarming behavior (yojN), genes involved in cellular respiration (arcB and frdA) and genes which have no known functions (b1936, yhjH, tolA, rnhB, rep and mreB) cause 2 to 12-fold increase in biofilm formation when interrupted.

Genes involved in the synthesis or degradation of proteins (aroD, clpX, leuO, leuL, miaA, prfC, ptrB, rnpB) genes involved in carbon metabolism (fucA and rbsK), gene involved in nitrogen metabolism (glnE), gene involved in colanic acid synthesis (wcal), genes involved in molybdopterin synthesis (modA, modC and moaC), gene involved in adaptation to high sodium concentration (nhaA), gene involved in cellular respiration (yecK), genes involved in the biosynthesis of polysaccharide adhesin (ycdS, ycdR, ycdQ and ycdP), gene associated with ferritin encoding gene ftn (b1904) and genes which have no known functions (b2531, yedK, yjcC, yjjQ, ykgK, hscA and trs5_{—}7) cause 2 to 24-fold decrease in biofilm formation when interrupted

The genes of the invention and their corresponding polypeptides (proteins/enzymes) thus provide targets for the development of antibacterial and antibiofilm compounds, including antibacterial or antibiofilm compounds either inhibit or bind to the potential antibacterial or antibiofilm targets (products of genes). These compounds may be synthetic or natural. Thus, the present invention provides methods for identifying compounds which modulate biofilm formation, comprising identifying compounds which modulate the activity of a polynucleotide or polypeptide of the invention. The present invention also provides methods for identifying environmental factors which modulate biofilm formation, comprising identifying environmental factors which modulate the activity of a polynucleotide or polypeptide of the invention.

### DETAILED DESCRIPTION OF THE INVENTION

Biofilm formation is a complex developmental process (Danese, et. al., 2001). Bacteria have the ability to monitor the environment and make use of the information obtained. Environmental and surface conditions determine if biofilm development takes place; If conditions are favorable, changes in gene expression occur soon after surface interaction. It is known that bacteria in biofilms are phenotypic variations of their planktonic counterparts (Lamont, et. al., 1999). This biofilm-induced change is systematic and ongoing during the entire course of biofilm formation (Sauer, et. al., 2002). This study focuses on the molecular mechanisms that take place within the first 24 hours of biofilm formation. It has been shown that motility and surface attachment abilities are important in the initial stages of biofilm production by Escherichia coli (Jackson, et. al., 2002). Previous biofilm studies involving E. coli have isolated mutations that affect these processes (Pratt, et. al., 2002). Choosing a non-motile strain lacking type I fimbriae allowed this study to focus on the genes not previously known to be associated with biofilm formation.

Over half of the mutations that produced an increased amount of biofilm were located in genes involved with flagellar function or synthesis. Many of the mutations could result in relieving steric hindrance by reducing the number of flagella present. A reduction in flagellum synthesis occurs during biofilm maturation, indicating their motility may antagonize the process (Prigent-Combaret, et. al., 1999; Watnick, et. al., 2000). However, this would not explain the effect of the fliD mutation, which eliminates filament cap proteins. This protein is responsible for terminating the transport and polymerization of flagellin during flagellum growth. Disruption of dnaK and mdoG, both linked to flagellum synthesis (Shi, et. al., 1992; Kennedy, 1992), increased biofilm production as well. Although these mutations are found in a diverse group of flagellum genes, perhaps they reflect a common cause. It is known that cells sense a surface by inhibition of flagellar rotation expression (Davey, et. al., 2000; Alavi, et. al., 2001). The prevention of rotation has been linked to signal transduction and changes in cell gene expression (Davey, et. al., 2000). The background motB deletion in the transposon mutants render the flagella paralyzed (Pratt, et. al., 1998). Perhaps this motB mutation effects a signaling pathway that normally detects surface interaction through inhibition of flagellum movement. Mutations in the flagellum genes may have relieved this suppression.

A gene involved in the phosphorelay-signaling pathway (Takeda et. al.; 2001) was interrupted in a biofilm up mutant. This gene, yojN, is part of a signaling pathway involved in modulating swarming behavior and colanic acid synthesis. It has been demonstrated that this system regulates gene expression during colonization on a surface. Swarmer cell differentiation in Proteus mirabilis has been linked to changes in flagellar rotation at a surface or in viscous medium (Belas, et. al., 1998). Perhaps the phosphorelay signaling pathway is affected by the background motB deletion in the same manner as the flagellum genes.

Genes involved in lipopolysaccharide (LPS) core biosynthesis (rfaG, rfaP, and rfaQ) and the surface O-polysaccharide (wecB; Barua, et., al., 2002), were interrupted in 10 biofilm-up mutants. Studies have demonstrated LPS inhibits biofilm formation (Mireles, et. al., 2001). The length of the O antigenic polysaccharide of LPS inversely correlates with bacterial adhesion in several strains of gram-negative bacteria (Landini, et. al., 2002). Inactivation of the rfa operon leads to a deep rough phenotype, which can enhance bacterial adhesion to surfaces in some conditions.

It has been demonstrated that mutations in surface and membrane proteins lead to defects in surface attachment in several gram-negative bacteria (Sauer, et. al., 2001). The outer membrane of gram-negative bacteria has an important role in promoting responses to environmental change (Otto, et. al., 2001). In nonfimbriated E. coli, inhibition of protein synthesis and turnover resulted in a decrease of outer membrane proteins and surface attachment. Many of the mutations identified in this study have a role, directly or indirectly, in protein synthesis and degradation. aroD (involved in amino acid biosynthesis), clpX (encoding a serine protease/chaperone), leuO and leuL (involved in leucine biosynthesis), miaA (involved in tRNA modification), prfC (encoding peptide chain release factor 3), and in particular ptrB (encoding a protease) and rnpB (encoding RNase P) led to decreases in biofilm development when interrupted.

Protein analysis has demonstrated changes in protein levels as early as 8 hours after attachment (Sauer, et. al., 2002). An increase in proteins involved in metabolic pathways such as cofactor biosynthesis, carbon catabolism and amino acid metabolism has been shown. Genes involved in carbon catabolism, fucA (encoding for L-fuculose-1-phosphate aldolase) and rbsK (encoding for ribokinase), were interrupted in 2 biofilm down mutants. A gene involved in the regulation of nitrogen consumption (glnE), when interrupted, caused a 2-fold decrease in biofilm production. wcaI created a decrease in biofilm by 24-fold when disrupted. The product of wcai is an unclassified glycosyl transferase known to be involved in colonic acid synthesis. Glycosyl transferases are involved in a variety of metabolic pathways. Genes involved in molybdopterin biosynthesis (modA, modC and moaC) were identified in three biofilm-down mutants. The genes of the moa operon as well as modA and modB are required for the first step in molybdopterin synthesis (Anderson, et. al., 2000). Molybdoenzymes are important in many bacterial metabolic pathways.

The environment within a biofilm is diverse and unlike the environment encountered by the free-living bacteria. Bacteria approaching a surface can encounter an increase in organic and inorganic ion concentrations, lower pH levels and changes in osmolarity (Prigent-Combaret, et. al., 1999). Surface attached E. coli have higher intracellular K⁺ levels than their planktonic counterparts after 10 hours of incubation, indicating attached bacteria encounter higher-osmolarity conditions. Studies have shown that membrane proteins principally involved in molecular transport and adaptation increase following adhesion (Sauer, et. al., 2002). nhaR, a gene involved in regulating nhaA, which encodes a Na⁺/H⁺ antiporter (Dover, et. al., 2001), and osmC, encoding for an envelope protein of unknown function (Toesca, et. al., 2001), caused a decrease in biofilm when interrupted. nhaR positively regulates nhaA in a Na⁺ dependent manner. nhaA is necessary for adaptation to high sodium concentrations and alkaline pH (Dover, et. al., 2002). nhaR is needed for the induction of osmC during increased osmolarity (Toesca, et. al., 2001).

As the biofilm develops, microenvironments form (Watnick, et. al., 2000). Bacteria associated with single-species biofilms must alter gene expression to survive in their microenvironment. Microelectrode measurements show oxygen and pH levels fall, proximal to the surface. Three genes identified in this study are involved in cellular respiration. arcB is part of a two-component signal transduction system involved in regulation of over 30 operons (Kwon, et. al., 2000). This system allows E. coli to sense various respiratory conditions (Matsushika, et. al., 1998). Interruption of arcB lead to increased biofilm production. Disruption of frdA, involved in famerate reductase production, lead to an increase in biofilm. yecK gene, also called torY, encodes for a cytochrome-C type protein (Gon, et. al., 2000). When interrupted, yecK created a decrease of approximately 20-fold. This gene is part of a recently described respiratory system. Preliminary characterizations of this system indicate it is closely related to the Tor and Dor respiratory systems of E. coli (Gon, et. al., 2000).

Intercellular adhesion occurs in the formation of microcolonies and is necessary for mature biofilm development (Cramton, et. al., 1999). Twenty-two biofilm-down mutants had interrupted genes located within the ycd operon (ycdp, ycdQ, ycdR and ycdS). The resulting phenotypic changes ranged from 7- to 20-fold. Protein products from this operon currently have undefined functions. Two of the genes (ycdQ and ycdR) have protein products homologous to IcaA and IcaB in Staphylococcus aureus. This locus has been shown to mediate polysaccharide intercellular adhesin (PIA) synthesis (Cramton, et. al., 1999). The intercellular adhesin locus (ica) is required for biofilm formation in S. aureus and S. epidermidis.

One gene whose disruption caused a 24-fold decrease in biofilm was b1904. Currently this gene's function is unknown, however it is directly upstream from the ferritin encoding gene ftn. Possibly, interruption of this gene created a polar effect with ftn. Previous research has indicated that iron may be an important factor in the initiation of biofilm formation in Pseudomonas aerugionsa (O'Toole, et. al., 1998).

Several of the genes identified in this study have no known function or homologue of known function. Disruption of b2531, yedK, yjcC, yjjQ and ykgK resulted in a decrease in biofilm production, while interruption of b1936 and yhjH created an increase. The effect of disruption of yjjQ was particularly notable. The observations in this study might facilitate future studies that characterize these genes by giving insight into the function their products have. A Current literature review yielded no beneficial information regarding the possible correlation between the phenotypic changes seen concerning biofilm formation and the interruption of trs5_{—}7, hscA, mreB, rep, rnhB and tolA. Therefore, no details are known of these genes' involvement with this process.

Of the sixty-one different genes identified, only those involved in motility are known to be associated with biofilm production. Interestingly, interruption of these genes created a change in the biofilm phenotype contrary to what has been seen in published research where disruption of motility decreased biofilm formation. Transduction of four gene mutations (nhaR, leuO, yjjQ and yojN) identified in this study into wild type E. coli K12 MG1655 created changes in biofilm formation. However, the mutations produced less of an impact in the wild type than with TRMG F/M. Complementation of the nhaR interruption using the plasmid construct pCRnhaR confirmed that this gene has an important role in biofilm formation.

Screening Assays Using the Identified Genes, Gene Products.

The genes and gene products identified in this study represent immediate targets for screens that would identify additional influences on biofilm development. A variety of existing procedures can be used to develop such procedures. For example, reporter constructs can be prepared for any gene of interest, which will allow the identification and measurement of genetic or environmental influences on the expression of that gene. A widely employed example of this is the use of 'lacZ transcriptional or translational fusions (example: Romeo et al., 1990; 1993). First, a fusion gene is created in which the regulatory elements that are located within the 5′-region of the gene are fused to the coding region of lacZ. This construct will express beta-galactosidase under the regulatory control of the gene of interest. Next, one can create mutants, either by directed or random procedures that alter the expression of the gene of interest. Screening with X-gal or on McConkey agar will allow mutations that influence the expression of the gene of interest.

The same kind of gene fusion permits environmental influences on gene expression to be assessed, including growth conditions and the presence of inhibitory agents or chemicophysical stresses. In the case of genes that are essential for biofilm formation, agents that inhibit biofilm formation by blocking the expression may be identified. An advantage of this kind of compound would be that it should not kill cells, but only alter their ability to form biofilm. Thus, resistances that are typical for drugs that kill or inhibit growth may not develop. Of course, many different gene expression reporters can be similarly employed, including antibiotic markers, such as chloramphenicol acetyl-transferase (cam) or agents that allow immediate visualization of effects, such as green fluorescent protein (gfp). Factors that influence the production of proteins that are encoded by biofilm-related genes can also be assessed, e.g. by immunological (Western) blotting (e.g. Liu et al., 1997).

Thus, the present teachings provide methods for screening compounds to identify those which enhance (agonist) or block (antagonist) the action of subject polypeptides or polynucleotides, and thus the formation of biofilms. Drug screening assays are made effective by use of the control regions of the genes described in the present invention or part of it, in a host based DNA-protein interaction assay.

For example, to screen for agonists or antagonists, a synthetic reaction mix, a cellular compartment, such as a membrane, cell envelope or cell wall, or a preparation of any thereof, may be prepared from a cell that expresses a molecule that binds a subject polynucleotide. The preparation is incubated with labeled polynucleotide in the absence or the presence of a candidate molecule which may be an agonist or antagonist. The ability of the candidate molecule to bind the binding molecule is reflected in decreased binding of the labeled ligand.

Effects of potential agonists and antagonists may by measured, for instance, by determining activity of a reporter system following interaction of the candidate molecule with a cell or appropriate cell preparation, and comparing the effect to a baseline (control) measurement.

Reporter systems that may be useful in this regard include, but are not limited to, calorimetric labeled substrate converted into product, a reporter gene that is responsive to changes in elongase enzyme activity, and binding assays known in the art.

Another example of an assay for antagonists is a competitive assay that combines a subject polypeptide and a potential antagonist with membrane-bound subject polypeptide-binding molecules, recombinant subject polypeptide binding molecules, natural substrates or ligands, or substrate or ligand mimetics, under appropriate conditions for a competitive inhibition assay. A subject polypeptide can be labeled, such as by radioactivity or a calorimetric compound, such that the number of subject polypeptide molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, peptides, polypeptides, such as closely related proteins or antibodies that bind the same sites on a binding molecule, without inducing subject polypeptide-induced activities, thereby preventing the action of the subject polypeptide by excluding the subject polypeptide from binding. Potential antagonists include antisense molecules (Okano et al., 1988, EMBO J., 7: 3407-3412). Potential antagonists include compounds related to and derivatives of the subject polypeptides. Potential antagonists may be selected from the group consisting of small organic molecules, peptides, polypeptides, antisense molecules, oligonucleotides, polynucleotides, fatty acids, and chemical and functional derivatives thereof. Selective modulators may include, for example, antibodies and other proteins or peptides which specifically bind to the subject polypeptide or polynucleotide, oligonucleotides which specifically bind to the subject polypeptide (see Patent Cooperation Treaty International Publication No. WO93/05182 which describes methods for selecting oligonucleotides which selectively bind to target biomolecules) or the subject polynucleotide (e. g., antisense oligonucleotides) and other non-peptide natural or synthetic compounds which specifically bind to the subject polynucleotide or polypeptide. Mutant forms of the subject polynucleotide which alter the activity of the subject polypeptide or its localization in a cell are also contemplated. Crystallization of recombinant subject polypeptides alone and bound to a modulator, analysis of atomic structure by X-ray crystallography, and computer modeling of those structures are methods useful for designing and optimizing non-peptide selective modulators. See, for example, Erickson et al., 1992, Ann. Rep. Med. Chez., 27: 271-289 for a general review of structure-based drug design.

Definitions

To facilitate a complete understanding of the invention, the terms defined below have the following meaning:

Biofilm refers to a community of microorganisms growing on a surface (inert or living) enclosed in an exopolysaccharide matrix.

Compositions include genes, proteins, polynucleotides, peptides, compounds, drugs, and pharmacological agents.

Disorder as used herein refers to derangement or abnormality of structure or function. Disorder includes disease.

Gene refers to a nucleic acid molecule or a portion thereof, the sequence of which includes information required for the production of a particular protein or polypeptide chain. The polypeptide can be encoded by a full-length sequence or any portion of the coding sequence, so long as the functional activity of the protein is retained. A gene may comprise regions preceding and following the coding region as well as intervening sequences (introns) between individual coding sequences (exons). A “heterologous” region of a nucleic acid construct (i.e. a heterologous gene) is an identifiable segment of DNA within a larger nucleic acid construct that is not found in association with the other genetic components of the construct in nature. Thus, when the heterologous gene encodes a biofilm gene, the gene will usually be flanked by a promoter that does not flank the structural genomic DNA in the genome of the source organism.

Host system may comprise a cell, tissue, organ, organism or any part thereof, which provides an environment or conditions that allow for, or enable, transcription and/or transcription.

Identity, similarity, homology or homologous, refer to relationships between two or more polynucleotide sequences, as determined by comparing the sequences. In the art, identity also means the degree of sequence relatedness between polynucleotide sequences, as the case may be, as determined by the match between strings of such sequences. Both identity and similarity can be readily calculated (Lesk A. M., ed., 1988, Computational Molecular Biology, Oxford University Press, NY; Smith D. W., ed., 1993, Biocomputing: Informatics and Genome Project, Academic Press, NY; Griffin A. M. and Griffin H. G., eds., 1994, Computer Analysis of Sequence Data, Part 1, Humana Press, NJ; von Heijne G., 1987, Sequence Analysis in Molecular Biology, Academic Press, NY and Gribskov M. and Devereux J., eds., 1991, Sequence Analysis Primer, M Stockton Press, NY). While there exist a number of methods to measure identity and similarity between two polynucleotide sequences, both terms are well known to skilled artisans (von Heijne G., 1987, Sequence Analysis in Molecular Biology, Academic Press, NY; Gribskov M. and Devereux J., eds., 1991, Sequence Analysis Primer, M Stockton Press, NY and Carillo H. and Lipman D., 1988, SIAM J. Applied Math., 48: 1073). Methods commonly employed to determine identity or similarity between sequences include, but are not limited to those disclosed in Carillo H. and Lipman D., 1988, SIAM J. Applied Math., 48: 1073. Methods to determine identity and similarity are codified in computer programs. Computer program methods to determine identity and similarity between two sequences include, but are not limited to, GCG program package (Devereux et al., 1984, Nucl. Acid Res., 12: 387-395), BLASTP, BLASTN and FASTA (Altschul et al., 1990, J. Molec. Biol., 215: 403-410).

Isolated means altered “by the hand of man” from its natural state; i.e., that, if it occurs in nature, it has been changed or removed from its original environment, or both. For example, a naturally occurring polynucleotide naturally present in a living organism in its natural state is not “isolated,” but the same polynucleotide separated from coexisting materials of its natural state is “isolated”, as the term is employed herein. As part of or following isolation, such polynucleotides can be joined to other polynucleotides, such as DNA, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNA still would be isolated, as the term is used herein, because they would not be in their naturally occurring form or environment. Similarly, the polynucleotides may occur in a composition, such as a media formulations, solutions for introduction of polynucleotides, for example, into cells, compositions or solutions for chemical or enzymatic reactions, for instance, which are not naturally occurring compositions, and, therein remain isolated polynucleotides within the meaning of that term as it is employed herein.

Mutation. A “mutation” is any detectable change in the genetic material. A mutation can be any (or a combination of) detectable, unnatural change affecting the chemical or physical constitution, mutability, replication, phenotypic function, or recombination of one or more deoxyribonucleotides; nucleotides can be added, deleted, substituted for, inverted, or transposed to new positions with and without inversion. Mutations can occur spontaneously and can be induced experimentally by application of mutagens or by site-directed mutagenesis. A mutant polypeptide can result from a mutant nucleic acid molecule.

Nucleic acid construct refers to any genetic element, including, but not limited to, plasmids and vectors, that incorporate polynucleotide sequences. For example, a nucleic acid construct may be a vector comprising a promoter or control region that is operably linked to a heterologous gene.

Operably linked as used herein indicates the association of a promoter or control region of a nucleic acid construct with a heterologous gene such that the presence or modulation of the promoter or control region influences the transcription of the heterologous gene, including genes for reporter sequences. Operably linked sequences may also include two segments that are transcribed onto the same RNA transcript. Thus, two sequences, such as a promoter and a “reporter sequence” are operably linked if transcription commencing in the promoter produces an RNA transcript of the reporter sequence.

Plasmids. Starting plasmids disclosed herein are either commercially available, publicly available, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention.

Polynucleotides(s) of the present invention may be in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded polynucleotides may be the coding strand, also known as the sense strand, or it may be the non-coding strand, also referred to as the anti-sense strand. Polynucleotides generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded, or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide also includes DNA or DNA that contain one or more modified bases. Thus, DNA or DNA with backbones modified for stability or for other reasons are “polynucleotides” as that term is intended herein. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. Polynucleotides embraces short polynucleotides often referred to as oligonucleotide(s). It will also be appreciated that RNA made by transcription of this doubled stranded nucleotide sequence, and an antisense strand of a nucleic acid molecule of the invention or an oligonucleotide fragment of the nucleic acid molecule, are contemplated within the scope of the invention. An antisense sequence is constructed by inverting the sequence of a nucleic acid molecule of the invention, relative to its normal presentation for transcription. Preferably, an antisense sequence is constructed by inverting a region preceding the initiation codon or an unconserved region. The antisense sequences may be constructed using chemical synthesis and enzymatic ligation reactions using procedures known in the art.

Promoter refers to a nucleic acid sequence comprising a DNA regulatory element capable of binding RNA polymerase directly or indirectly to initiate transcription of a downstream (3′ direction) gene. In accordance with the present invention, a promoter of a nucleic acid construct that includes a nucleotide sequence, wherein the nucleotide sequence may be linked to a heterologous gene such that the induction of the promoter influences the transcription of the heterologous gene.

Purified. A “purified” protein or nucleic acid is a protein or nucleic acid preparation that is generally free of contaminants, whether produced recombinantly, chemically synthesized or purified from a natural source.

Recombinant refers to recombined or new combinations of nucleic acid sequences, genes, or fragments thereof which are produced by recombinant DNA techniques and are distinct from a naturally occurring nucleic acid sequence

Regulatory element refers to a deoxyribonucleotide sequence comprising the whole, or a portion of, a nucleic acid sequence to which an activated transcriptional regulatory protein, or a complex comprising one or more activated transcriptional regulatory proteins, binds so as to transcriptionally modulate the expression of an associated gene or genes, including heterologous genes.

Reporter gene is a nucleic acid coding sequence whose product is a polypeptide or protein that, is not otherwise produced by the host cell or host system, or which is produced in minimal or negligible amounts in the host cell or host system, and which is detectable by various known methods such that the reporter gene product may be quantitatively assayed to analyse the level of transcriptional activity in a host cell or host system. Examples include genes for luciferase, chloramphenicol acetyl transferase (CAT), beta-galactosidase, secreted placental alkaline phosphatase and other secreted enzymes.

Silencer refers to a nucleic acid sequence or segment of a DNA control region such that the presence of the silencer sequence in the region of a target gene suppresses the transcription of the target gene at the promoter through its actions as a discrete DNA segment or through the actions of trans-acting factors that bind to these genetic elements and consequently effect a negative control on the expression of a target gene.

Stringent hybridization conditions are those which are stringent enough to provide specificity, reduce the number of mismatches and yet are sufficiently flexible to allow formation of stable hybrids at an acceptable rate. Such conditions are known to those skilled in the art and are described, for example, in Sambrook et al., 1989, Molecular Cloning, 2nd Edition, Cold Spring Harbor Laboratory Press, Cold Spring Harbour, N.Y. or Ausubel et al., 1994-, Current Protocols in Molecular Biology, John Wiley & Sons, NY. By way of example only, stringent hybridization with short nucleotides may be carried out at 5-10° C. below the T_{M} using high concentrations of probe such as 0.01-1.0 pmole/ml. Preferably, the term “stringent conditions” means hybridization will occur only if there is at least 95% and preferably at least 97% identity between the sequences.

Tag refers to a specific short amino acid sequence, or the oligonucleotide sequence that encodes it, wherein said amino acid or nucleic acid sequence may comprise or encode, for example, a c-myc epitope and/or a string of six histidine residues recognizable by commercially available antibodies. In practice, a tag facilitates the subsequent identification and purification of a tagged protein.

Tagged protein as used herein refers to a protein comprising a linked tag sequence. For example, a tagged protein includes a biofilm polypeptide linked to a c-myc epitope and six histidine residues at the carboxyl terminus of the amino acid sequence.

Test compounds as used herein encompass small molecules (e.g. small organic molecules), pharmacological compounds or agents, peptides, proteins, antibodies or antibody fragments, and nucleic acid sequences, including DNA and RNA sequences.

Transfection refers to a process whereby exogenous or heterologous DNA (i.e. a nucleic acid construct) is introduced into a recipient eukaryotic host cell. Therefore, in eukaryotic cells, the acquisition of exogenous DNA into a host cell is referred to as transfection. In prokaryotes and eukaryotes (for example, yeast and mammalian cells) introduced DNA may be maintained on an episomal element such as a plasmid or integrated into the host genome. With respect to eukaryotic cells, a stably transfected cell is one in which the introduced DNA has become integrated into a chromosome so that it is inherited by daughter cells through chromosome replication. This stability is demonstrated by the ability of the eukaryotic cell to establish cell lines or clones comprised of a population of daughter cells containing the introduced DNA.

Transformation refers to a process whereby exogenous or heterologous DNA (i.e. a nucleic acid construct) is introduced into a recipient prokaryotic host cell. Therefore, in prokaryotic cells, the acquisition of exogenous DNA into a host cell is referred to as transformation. Transformation in eukaryotes refers to the conversion or transformation of eukaryotic cells to a state of unrestrained growth in culture, resembling a tumorigenic condition. In prokaryotes and eukaryotes (for example, yeast and mammalian cells) introduced DNA may be maintained on an episomal element such as a plasmid or integrated into the host genome. With prokaryotic cells, a stably transformed bacterial cell is one in which the introduced DNA has become integrated into a chromosome so that it is inherited by daughter cells through chromosome replication. This stability is demonstrated by the ability of the prokaryotic cell to establish cell lines or clones comprised of a population of daughter cells containing the introduced DNA.

Transfection/transformation as used herein refers to a process whereby exogenous or heterologous DNA (e.g. a nucleic acid construct) has been introduced into a eukaryotic or prokaryotic host cell or into a host system.

Variant(s) of polynucleotides are polynucleotides that differ in nucleotide sequence from another, reference polynucleotide. A “variant” of a protein or nucleic acid is meant to refer to a molecule substantially similar in structure and biological activity to either the protein or nucleic acid. Thus, provided that two molecules possess a common activity and can substitute for each other, they are considered variants as that term is used herein even if the composition or secondary, tertiary, or quaternary structure of one of the molecules is not identical to that found in the other, or if the amino acid or nucleotide sequence is not identical. Generally, differences are limited so that the nucleotide sequences of the reference and the variant are closely similar overall and, in many regions, identical. Changes in the nucleotide sequence of the variant may be silent. That is, they may not alter the amino acids encoded by the polynucleotide. Where alterations are limited to silent changes of this type a variant will encode a polypeptide or polynucleotide with the same amino acid sequence as the reference. Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide or polynucleotide encoded by the reference sequence.

Vector. A plasmid or phage DNA or other DNA sequence into which DNA can be inserted to be cloned. The vector can replicate autonomously in a host cell, and can be further characterized by one or a small number of endonuclease recognition sites at which such DNA sequences can be cut in a determinable fashion and into which DNA can be inserted. The vector can further contain a marker suitable for use in the identification of cells transformed with the vector. Markers, for example, are tetracycline resistance or ampicillin resistance. The words “cloning vehicle” are sometimes used for “vector.”

The terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

The present invention is further described and will be better understood by referring to the working examples set forth below. These non-limiting examples are to be considered illustrative only of the principles of the invention. Since numerous modifications and changes will readily occur to those skilled in the art, it is not desired to limit the invention to the exact construction and operation shown and described. Accordingly, all suitable modifications and equivalents may be used and will fall within the scope of the invention and the appended claims.

### EXAMPLES

The present invention is further described by the following examples. These examples, while illustrating certain specific aspects of the invention, do not portray the limitations or circumscribe the scope of the disclosed invention.

### Example 1

### Generation of Library of Mutants and Screening for Biofilm Formation

Escherichia coli K12, substrain MG1655 csrA::kan ΔmotB ΔfimB-H (designated as TRMG F/M in this study) was used as the parent strain in generating the library of mini Tn10::cam transposon mutants. The mutations that resulted in altered biofilm phenotypes as compared to the parent were then transduced either back into the parent or the closely related strain E. coli MG1655 csrA::kanR (designated in this study as TRMG1655). The lambda phage permissive host E. coli LE392 was used in the preparation of the λ lysate for the transposon mutagenesis. Wild type E. coli K12, substrain MG1655 was used in the transduction and complementation studies involving four genes whose interruption resulted in a change in biofilm formation as compared to the parent. E. coli TOP 10 electrocompetent cells (Invitrogen, Carlsbad, Calif.) were used in the cloning of selected genes for the complementation studies. These competent cells contain no lacI repressor gene, therefore, IPTG is not needed for blue/white screening of recombinants. The phage λ NK1324 used in the transposon mutagensis contains the miniTn10::cam resistance marker, enabling selection of the transposon mutants by resistance to the antibiotic chloramphenicol (Kleckner, et. al., 1991). Candidate transposon mutants had their mutations confirmed by transduction using the P1vir phage. This bacteriophage is strictly lytic and forms only clear plaques. The plasmid pCR2.1 (Invitrogen, Carlsbad, Calif.), used to clone selected genes for complementation, carries the ampicillin resistance gene as well as lacZα gene. It is supplied linearized with single 3^{/} end thymidine overhangs and has topoisomerase covalently bound to the 3^{/} end (see Table 1 for a summary of these mutants).

TABLE 1
Bacterial strains, phages and plasmids

| Strains, phages | | |
|---|---|---|
| and plasmids | Source | Characteristics |
| E. coli | | |
| K12 strains | | |
| MG1655 | Michael Cashel | Wild type |
| TRMG1655 | Laboratory strain | MG1655 csrA::kanR |
| TRMG F/M | Laboratory strain | TRMG1655Δ motB ΔfimB-H |
| | | uvrC 279::Tn10 |
| TOP10 | Invitrogen | electrocompetent strain |
| Bacteriophages | | |
| λNK1324 | ATCC | miniTn10::camR |
| P1vir | Carol Gross | Strictly lytic P1, clear plaques |
| Plasmids | | |
| pCR2.1 | Invitrogen | TA cloning vector, |
| | | Ampicillcin resistant |

Transposon Mutagenesis

Protocols followed for transposon mutagenesis are based on the published procedures (Kleckner, et. al., 1991).

Preparation of λ NK1324 Lysate

The permissive host strain E. coli LE392 was grown to saturation in Tryptone B1 Broth with maltose and magnesium (TBMM) medium at 37° C. overnight. A serial dilution was made of the ATCC λ NK1324 stock in TMG buffer (1.2 g Tris base, 2.46 g MgSO₄.7H₂O, 0.1 g gelatin, 1 liter distilled water, adjust pH to 7.4). Aliquots (0.1 ml) of each dilution were added to 0.1 ml of the overnight LE392 culture. The resulting mixture was incubated for 20 minutes in a 37° C. water bath. Tryptone Broth (TB) top agar (3.0 ml), at a temperature of 45° C., was added to each tube and mixed well. This mixture was then poured atop a Tryptone B1 (TB1) bottom agar plate, allowed to solidify and incubated at 39.5 to 42° C. overnight (7-8 hours). Large single plaques were picked and individually placed in microcentrifuge tubes containing 0.2 ml LB broth plus 0.01M MgSO₄. The mixtures were vortexed and allowed to stand at room temperature for 10 minutes. In a small test tube, 0.1 ml of the dissolved plaque mixture was mixed with 0.1 ml of an overnight LE392 culture. This was incubated in a 37° C. water bath for 20 minutes. Molten TB top agar (3 ml at 45° C.) was added and gently vortexed. This mixture was then poured atop a TB1 bottom agar plate, allowed to solidify and incubated at 39.5 to 42° C. overnight (7-8 hours). After incubation, the entire top agar surface was removed and placed into a sterile centrifuge tube. LB broth (1 ml) plus 0.01M MgSO₄ was used to rinse the plate. This rinse was added to the top agar surface scraping. To this, five drops of chloroform were added. After vortexing, the mixture was centrifuged at 3000×g (J-21C centrifuge, Beckman coulter, Fullerton, Calif.) for 20 minutes at 4° C. The supernatant solution was recovered, and a drop of chloroform was added. It was stored at 4° C.

Titration of λ NK1324 Lysate

Serial dilutions of the phage lysate were prepared using TMG buffer. An overnight culture of LE392 (0.1 ml) was added to 0.1 ml of each dilution for final serial phage dilutions of 10⁻⁷ to 10⁻⁸. The mixtures were then incubated for 20 minutes in a 37° C. water bath. Molten TB top agar (3 ml at 45° C.) was mixed into the samples. This mixture was poured atop a TB1 bottom agar plate and allowed to solidify. The plates were incubated at 40° C. overnight. The plaques were counted and the pfu/ml of the lysate calculated.

Construction of Transposon Mutants

The parent strain TRMG F/M was subjected to insertion mutagenesis as follows. An overnight culture grown in TBMM was diluted 1:100 in TBMM for a total volume of 50 ml. This was incubated at 37° C. until the absorbance at 600 nm reached 1 (Ultraspec III, Pharmacia). The cells were then harvested by centrifugation at 7500×g and 4° C. (J-21C Beckman centrifuge). Cells were resuspended in 1 ml LB broth with 0.2% maltose. Concentrated cells (0.2 ml) were mixed with the λNK1324 lysate for a multiplicity of infection equal to 0.2. The mixture was incubated for 30 minutes at room temperature and then 90 minutes at 37° C. Infected cells (0.1 ml) were then plated on Kornberg agar containing 1% glucose, 2.5 mM sodium pyrophosphate and 25 μg/ml chloramphenicol. The plates were incubated overnight at 39° C. Colonies were picked and inoculated into wells on a 96 well microtiter tray containing Colony-Forming Antigen (CFA) medium plus 25 μg/ml chloramphenicol. The microtiter trays were incubated at 26° C. without shaking for 24 hours. The 24-hour cultures were then used to inoculate the corresponding wells of another microtiter tray containing CFA plus 25 μg/ml chloramphenicol for a final dilution of 1:100. These trays were then incubated at 26° C. without shaking for 24 hours. The total turbidity and quantity of biofilm formed was determined by the assay described below.

Biofilm Assay

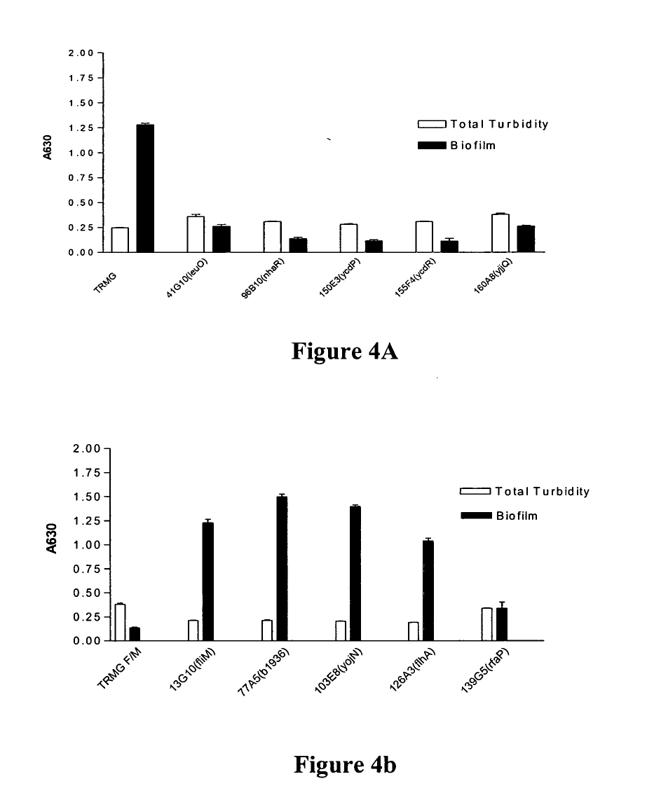

A 1:100 dilution of overnight culture and CFA plus 20 μg/ml chloramphenicol was used to inoculate wells of a 96-well microtiter tray in triplicate. The trays were incubated for 24 hours at 26° C. without shaking. Total turbidity of the wells was determined by measuring the absorbance at 630 nm. The amount of biofilm produced was determined by measuring the absorbance of crystal violet stained attached cells (Jackson et. al., 2002). This was accomplished by discarding the medium, rinsing the well three times with deionized water and staining the attached cells within the wells for one minute with crystal violet (41%, W/V). The crystal violet was discarded and the wells rinsed again three times with deionized water. After allowing the wells to air dry, the dye was solubilized with 100 μl of 33% acetic acid. The absorbance of resulting solution was measured at 630 nm using a Dynatech MXR microtiter plate reader (Dynatech, Chantilly, Va.)(see FIGS. 4A & B for results).

Transduction

Transduction was performed using P1vir lysate (described above) from transposon mutants that displayed altered biofilm production as compared to the parent, TRMG F/M (Miller et. al., 1972). Recipient strains for transduction were: TRMG1655 (for mutants that displayed a decrease in biofilm) and TRMG F/M (for mutants that displayed an increase in biofilm). Recipient strains were grown overnight in LB and 0.2% glucose broth (1 ml) plus 50 μg/ml kanamycin at 37° C. while rotating at 250 rpm. The overnight cells were harvested by centrifugation and resuspended in 1 ml MC buffer (0.1 M MgSO₄, 0.005 M CaCl₂). The resulting suspension was aerated for 15 minutes at 37° C. while rotating at 250 rpm. The reactions were incubated in a 37° C. water bath for 20 minutes. Sodium citrate buffer (1 M, 0.2 ml) was added and the contents mixed well. The cells were then plated to Kornberg agar containing 0.5% glucose and chloramphenicol (25 μg/ml). The plates were incubated overnight at 37° C. Transductants were re-streaked onto Kornberg agar containing 0.5% glucose and chloramphenicol (25 μg/ml) and incubated overnight. Colonies were tested for altered biofilm production as compared to the parent as previously described.

### Example 2

### Identification of Transposon Insertion Sites

Arbitrarily Primed Polymerase Chain Reaction

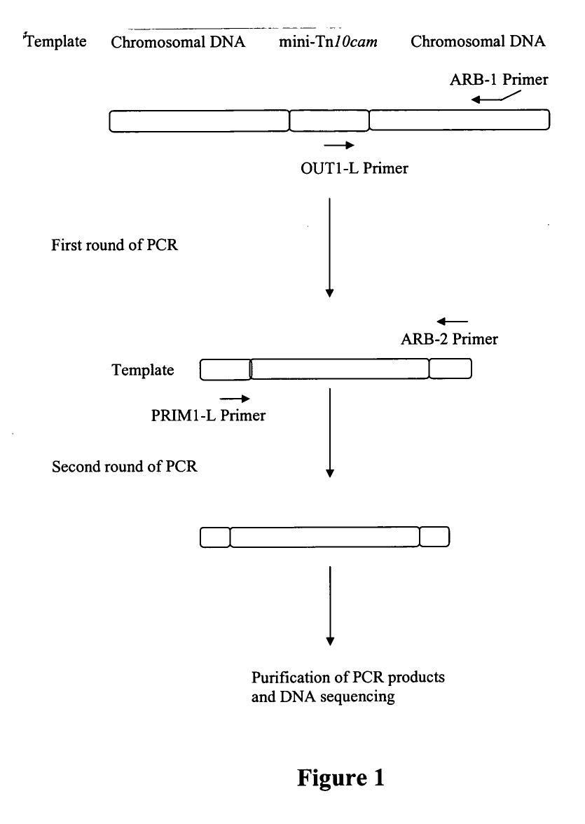

Arbitrarily primed polymerase chain reaction (PCR) was performed to amplify the chromosomal DNA flanking the transposon insertion sites (Pratt, et. a., 1998). Primers used in the first round were OUT1-L (designed to anneal to the one end of the insertion element) and ARB-1 (designed to anneal to arbitrary chromosomal sequences flanking the insertion). Primer PRIM1-L, used in the second round of the PCR, is designed to anneal to the same end of the insertion element as OUT1-L. However, PRIM1-L is specific for the extreme end, near the junction with the chromosome. ARB-2, used in the second round as well, is identical in sequence to the 5^{/} end of the ARB-1 primer (FIG. 1). Alternatively, DNA flanking the insertion sites not successfully amplified with the above primers was subjected to PCR using CAM-5IN and CAM-5OUT primers in conjunction with ARB-1 and ARB-2 primers described above. CAM-5IN and CAM-5OUT are designed to anneal to the opposite end of the insertion element in the same manner as OUT1-L and PRIM1-L. For primer sequences refer to Table 3.

The chromosomal template was prepared by inoculating LB broth (1 ml) plus 0.2% glucose and chloramphenicol (20 μg/ml) with ice scrapings from the desired stock. This was incubated overnight at 37° C. while rotating at 250 rpm. The overnight culture (200 μl) was centrifuged for 30 seconds at 16,000×g (MP4R centrifuge, International Equipment Co., Needham Heights, Mass.) and the supernatant discarded. The cells were resuspended in 100 μl sterile deionized water. The cell suspension was then boiled for 5 minutes at 95° C. and allowed to cool on ice for 2 minutes. Following centrifugation as described above, the supernatant containing the chromosomal DNA was used as the template for the amplification. The PCR reactions procedures are based on protocols from Invitrogen using Elongase® enzyme mix (Invitrogen, Carlsbad, Calif.) and standard reaction conditions for PCR (Sambrook, et. al., 2001). The enzyme mix consists of a mixture of Taq and Pyrococcus species GB-D thermostable DNA polymerase. The Elongase® comes packaged with a buffering system that allows optimization of the reaction with respect to the amount of [Mg²] used in the mixture. A PTC-100 programmable Thermal Cycler (MJ Research, Inc., Waltham, Mass.) was used to carry out the PCR reactions. The first round (25 μl total volume) of arbitrarily primed PCR was set-up as follows: 0.5 μl dNTP (10 mM), 0.25 μl OUT1-L primer (50 μM), 0.25 μl ARB1 primer (50 μM), 5 μl Buffer B (final [Mg²] 2.0 mM), 2.5 μl chromosomal DNA template, 16 μl sterile deionized water, 0.5 μl Elongase® enzyme mix. The solutions were gently mixed and placed into the Thermal Cycler. The first round conditions were: (1) 95° C. for 30 seconds, (2) 95° C. for 30 seconds, (3) 43° C. for 30 seconds, (4) 72° C. for 2 minutes, (5) Cycle to step 2 for 30 times, (6) 4° C. for 12 hours. The second round reaction (50 μl total volume) was set up as follows: 1 μl dNTP (10 mM), 0.5 μl ARB2 primer (50 μM), 0.5 μl 1-L primer (50 μM), 10 μl buffer B (final [Mg²⁺] 2.0 mM), 5 μl DNA template (first round product), 32 μl sterile deionized water, 1 μl Elongase® enzyme mix. The solutions were mixed and placed into the Thermal Cycler. The second round reaction conditions were: (1) 95° C., for 30 seconds, (2) 95° C., for 30 seconds, (3) 55° C. for 30 seconds, (4) 72° C. for 2 minutes, (5) cycle to step 2 for 30 times, (6) 4° C. for 12 hours. The PCR products were stored at −20° C.

TABLE 2
Sequence of Primers

| Primer Name | Sequence (5′ to 3′) |
|---|---|
| ARB-1 | GGCCACGCGTCGACTAGTACNNNNNNNNNNGAT |
| OUT1-L | CAGGCTCTCCCCGTGGAG |
| CAM-IN | AAAGTGCGGGTGATGC |
| ARB-2 | GGCCACGCGTCGACTAGTAC |
| PRIM1-L | CTGCCTCCCAGAGCCTG |
| CAM-OUT | CTGACGGGGTGGTGCGTAACGGC |
| nhaRF | CACTCGTGAGCGCTTACAGCCG |
| nhaRR | GATTCCTCTATTTATTCGCCCGC |
| leuOF | CTAATCAACGAGGAAAAAGGGAC |
| leuOR | AATCGGCTGAATCCCACAACTTAC |
| yjjQF | CTTCTGACACATGCAGTGGAGTTG |
| yjjQR | GCGATTGTACGCTGAAATGAAAGCAC |
| yojNF | ATGCCACTGCATACTGATTAACCC |
| yojNR | CGGTGCAAATGCCAGATAAGACACTAAC |
| M13UP | CAGGAAACAGCTATGAC |
| nhaR1 | AAACGTGAACTGATGCAAGGATCGC |
| nhaR2 | GATCATCGCTGGCTTGCTGATTGGC |
| nhaR3 | GATATTGTGAACTATCGCAAAGAATCC |
| nhaR4 | CGATGCACAATGCAATCTTCGTTGCC |

Gel Purification of PCR Products

PCR products were separated on 1% agarose gel using 0.5×TBE electrophoresis buffer (0.045 M Tris-borate, 0.001 M EDTA). Ethidium bromide was added to aid in visualization of the amplification product with UV light. The products were purified by gel extraction using QIAquick® Gel Extraction Kit following the manufacturers recommended protocol (QIAGEN, Valencia, Calif.). The concentration of the purified DNA was determined by the absorbance of a 1:10 dilution of the sample at 260 nm (GeneQuant III, Pharmacia Biotech, Piscataway, N.J.). Absorbance readings at 230 nm, 280 nm, and 320 nm were also determined to check for protein and organic contaminants. Samples were stored at −20° C.

Sequencing of Insertion Sites

After determining the correct concentration of DNA needed (2.5 ng/μl are needed per 100 base pairs), the purified amplification products were sent to the University of Arizona's DNA sequencing facility. This lab, managed by the Laboratory of Molecular and Systematics Evolution (LSME), uses a fluorescence-based DNA sequencing system. Dideoxynucleotides, each labeled with a distinct flourochrome, produce chain terminating reactions and labeled products. The products are analyzed and sequences determined using an ABI PRISM 3700 DNA Analyzer (Applied Biosystems Incorporated, Foster City, Calif.). The sequences are made available on their Internet web site (http://uofadna.arl.arizona.edu) and downloaded for further analysis.

Identification of Genes Interrupted by the Transposon

Identification of the insertion sites was made by performing BLAST searches. Basic Local Alignment Search Tool, or BLAST, is a program sponsored by the NCBI (National Center for Biotechnology Information). BLAST is a set of similarity search programs that are designed to search databases for matches to a query sequence (Altschul, et. al., 1990). The sequence information obtained from the University of Arizona was searched with this program against Escherichia coli K12 databases maintained at NCBI.

Complementation Studies

Four genes not previously known to be involved in biofilm formation, and whose interruption resulted in altered biofilm development, were chosen for complementation studies.

Transduction of E. coli MG1655

P1vir lysates from transposon mutants whose interrupted gene was chosen for complementation were used to co-transduce (previously described) the chloramphenicol resistance marker into E. coli K12 substrain MG1655. Colonies were picked and plated to Kornberg agar containing 0.5% glucose and chloramphenicol (25 μg/ml). After overnight growth, isolated colonies were used to inoculate LB broth plus 0.2% glucose (1 ml) with chloramphenicol (25 μg/ml) and incubated overnight at 37° C. while rotating at 250 rpm. Biofilm assays were performed and stock cultures prepared as previously described. The results of the biofilm assay were subjected to analysis of variance testing (ANOVA) to determine if changes seen were statistically significant as compared to the wild type.

Primer Design and PCR Conditions for Selected Genes

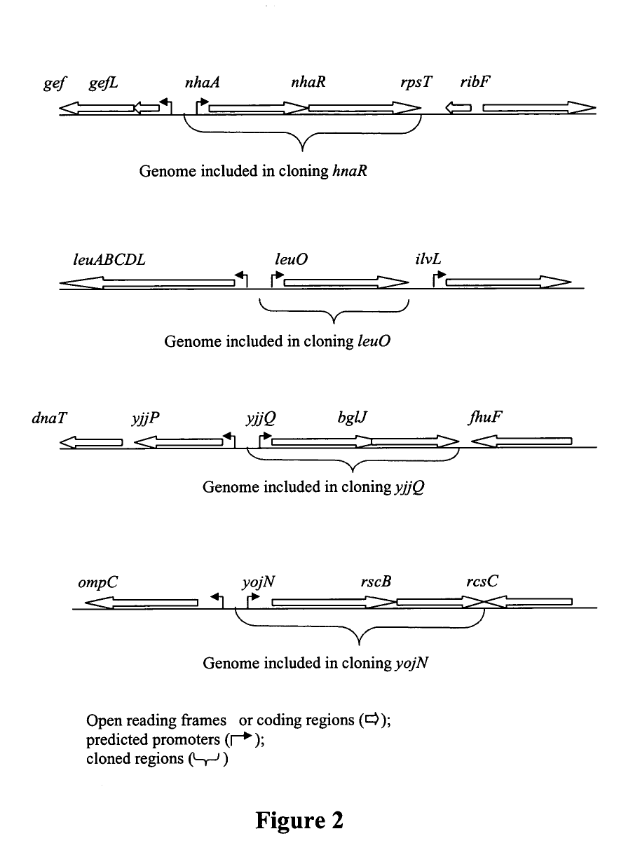

Primers were designed to include the predicted promoter region as well as the stop codons for each gene or operon selected (FIG. 2). PCR conditions and reaction mixtures are based on protocols from Invitrogen (Invitrogen, Carlsbad, Calif.), using Elongase® enzyme mix and standard reaction conditions for PCR (Sambrook, et. al., 2001). A manual hot start was used at the beginning of each PCR protocol to maximize the production of the desired PCR product. PCR was carried out using a PTC-100 programmable Thermal Cycler (MJResearch Inc., Waltham, Mass.). For primers used, refer to Table 2. A description of the PCR conditions for each gene or operon follows.
I. nhaR—The segment is 2.4 kb in length.
   PCR conditions: (1) 94° C., 30 seconds; (2) 94° C., 30 seconds; (3) 55° C., 30 seconds; (4) 68° C., 3 minutes; (5) cycle to step 2, thirty times; (6) 72° C., 10 minutes; (7) 4° C., 12 hours.
II. leuO—The segment is 1.6 kb in length.
   PCR conditions: (1) 94° C., 30 seconds; (2) 94° C., 30 seconds; (3) 55° C., 30 Seconds; (4) 68° C., 2 minutes; (5) cycle to step 2, thirty times; (6) 72° C., 10 minutes; (7) 4° C., 12 hours.
III. yjjQ—The segment is 1.6 kb in length.
   PCR conditions: (1) 94° C., 30 seconds; (2) 94° C., 30 seconds; (3) 57° C.; 30 seconds; (4) 68° C., 2 minutes; (5) cycle to step 2, thirty times; (6) 72° C., 10 minutes; (7) 4° C., 12 hours.
IV. yojN—The segment is 4.1 kb in length ion.
   PCR conditions: (1) 94° C., 30 seconds; (2) 94° C., 30 seconds; (3) 55° C., 30. seconds; (4) 68° C., 4 minutes; (5) cycle to step 2, forty times; (6) 72° C., 15 minutes; (7) 4° C., 12 hours.

The PCR reactions were carried out in 50 μl total volume amounts using the following reagents: 1 μl dNTP (10 mM), 0.5 μl forward primer (50 μM), 0.5 μl reverse primer (50 μM), 1 μl chromosomal DNA template (490 ng/μl), 10 μl buffer B (final [Mg²] 2.0 mM), 36 μl sterile deionized water, 1 μl Elongase® enzyme mix.

Purification of Long PCR Products

PCR products for cloning were gel purified as recommended by TOPO PCR cloning kit for long PCR products (Invitrogen, Carlsbad, Calif.). Briefly, PCR products were separated on a 0.8% agarose gel made with 1×TAE buffer (0.04 M Tris-acetate and 0.001 M EDTA), containing 2 mg/ml Crystal Violet solution for visualization of DNA. Eight microliters of 6× Crystal Violet Loading Buffer (Invitrogen, Carlsbad, Calif.) were used to load the PCR product onto the gel. After electrophoresis, the PCR product was excised from the gel and placed into a sterile microcentrifuge tube, weighted, and 2.5 volumes of 6.6 M sodium iodide were added and mixed by vortexing. The sample was then incubated at 50° C. until the agarose completely melted. Binding buffer (Invitrogen, Carlsbad, Calif.) was mixed in (1.5 volumes). The suspension was loaded onto a S.N.A.P. (Invitrogen, Carlsbad, Calif.) purification column. DNA was bound to the membrane in the column by centrifugation at 2500×g for 30 seconds (MP4R centrifuge, International Equipment Co.). To ensure the entire amount of DNA was harvested from the solution, the filtrate was poured back into the column and this step repeated two times. The column was washed twice with 400 μl of Final Wash (Invitrogen, Carlsbad, Calif.), containing 75% ethanol and 400 mM NaCl. After allowing the column membrane to air dry, the DNA was eluted by applying 40 μl TE buffer and centrifuging at 16,000×g for one minute (MP4R centrifuge, International Equipment Co.). Absorbance readings of a 1:10 dilution at 260 nm were made to determine the concentration of the purified PCR products (GeneQuantII, Pharmacia Biotech). The DNA was stored at −20° C.

Ligation

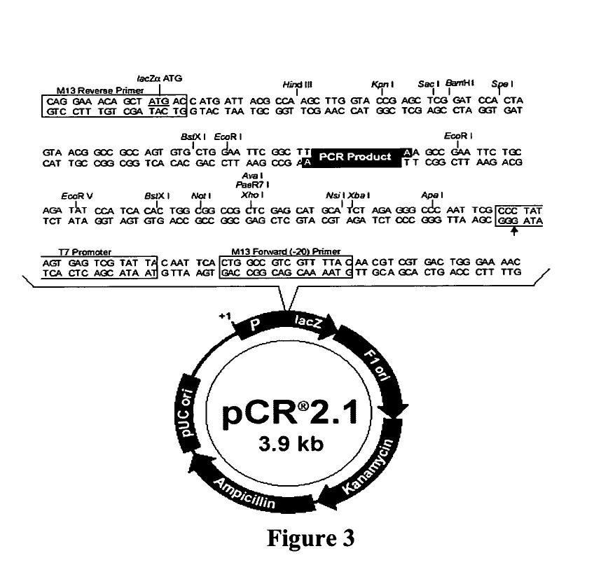

The purified PCR products were ligated into the pCR2.1 plasmid vector (FIG. 3) using the TOPO TA cloning kit as described by the manufacturer (Invitrogen, Carlsbad, Calif.).

Preparation of Electrocompetent Cells

Overnight cultures (2.5 ml) were used to inoculate 500 ml of LB broth plus 0.2% glucose and 20 μg/ml chloramphenicol in a 2000 ml flask. This was incubated at 37° C. while rotating at 250 rpm until an optical density at 600 nm reached approximately 0.8.

(Ultraspec III, Pharmacia Biotech). Aliquots of the cells (250 ml) were transferred to chilled 250 ml sterile centrifuge bottles (Nalgene, Co.) and pelleted by centrifuging at 4° C. for 15 minutes at 7520×g (J-21C Beckman centrifuge). The cells were resuspended in 250 ml ice cold 10% glycerol and pelleted again for 15 minutes as described above. After decanting the supernatant, the cells were resuspended in 125 ml of ice cold 10% glycerol. Repeating the above centrifugation, the cells were pelleted a third time and resuspended in 62.5 ml ice cold 10% glycerol. The cell suspensions were combined into one 125 ml portion and pelleted as previously described. Each cell pellet was suspended in 1.5 ml ice cold 10% glycerol. Aliquots (150 μl) of the suspensions were dispensed into sterile 1.5 ml microcentrifuge tubes and stored at −80° C.

Transformation

Electrocompetent cells, prepared as described above, or E. coli TOP electrocompetent cells (Invitrogen, Carlsbad, Calif.) were thawed on ice and transformed by mixing 2-3 μl of DNA with 50 μl of the thawed cells. The mixture was then transferred to a pre-chilled 0.1 cm electrode gap electroporation cuvette (Bio-Rad Laboratories, Hercules, Calif.) and electroporated using a Gene Pulser II Apparatus (Bio-Rad Laboratories). Electroporation settings were 1.8 kilovolts, 25 microfaradays, and 200 ohms with a maximum time of 4.5 to 5 milliseconds. Cells were immediately recovered after electroporation in 1 ml room temperature SOC medium [2% Tryptone, 0.5% yeast extract, 10 mM NaCl, 2.5 mM KCl, 10 mM MgCl₂, 10 mM MgSO₄, and 20 mM glucose (Invitrogen, Carlsbad, Calif.)]. Cell suspensions were transferred to a 15 ml culture tube and incubated for at least one hour at 37° C. with gentle rotation for aeration. Various volumes (20 μl, 50 μl, 80 μl, 100 μl and 150 μl) of the electroporated cell suspensions were used to inoculate LB or Komberg agar plates containing the carbenicillin (100 μg/ml). X-gal (40 μl, 40 mg/ml) reagent was added to the media for the blue/white screening of the pCR2.1 vector constructs containing the cloned inserts. The plates were incubated overnight at 37° C.

Selection of Transformed Cells

Colonies displaying the correct selection criteria, as indicated below, were grown overnight in 1 ml LB broth containing 0.2% glucose and 100 μg/ml carbenicillin. Stocks of the overnight cultures were prepared as previously described. Presumptive transfonnants having vector constructs with the cloned inserts were identified by blue/white screening and resistance to carbenicillin. Transformed transposon mutants or transductants were determined by resistance to carbenicillin.

Isolation of Plasmid Constructs from E. coli TOP 10 Cells

Mini-preparations of plasmid from E. coli TOP 10 cells were obtained using Quantum Prep® Plasmid miniprep kit (BioRad Laboratories) as described by the manufacturer. Alternatively, mini-preparations were made by centrifuging an overnight culture (1.5 ml) at 10,000×g for 1-2 minutes (MP4R, International Equipment Co.). The cell pellets were suspended in 150 μl ice cold SET buffer. Freshly made lysis buffer (350 μl; 20 μl 10N NaOH, 1 ml 10% SDS, 8.8 ml sterile deionized water) was added. The suspension was gently mixed and placed on ice for 10 minutes. Ice cold KAC (250 μl; 3 M K+, 5 M Acetate) was added, the mixture was inverted several times and placed on ice for 10 minutes. After centrifuging the tubes for 10 minutes at 16,000×g and 4° C. (MP4R, International Equipment Co.), the supernatant solution was transferred to a clean tube. An equal amount of room temperature 100% isopropanol was added and the tubes centrifuged for 5 minutes at 16,000×g. After carefully removing the supernatant, the pellet was resuspended in 200 μl TE buffer (10 mM Tris Cl, 1 mM EDTA pH 8.0). RNaseA (2 μl; 20 ng/ml) was added and the mixture was incubated at 37° C. for 30 minutes. Phenol:chloroform (200 μl, 1:1 ratio) was added. After mixing and centrifugation (16,000×g for 10 minutes at 4° C.), the upper phase was collected and transferred to a clean tube. An equal volume of chloroform was added and the mixture centrifuged as previously described. The upper phase was transferred to a clean tube and 4 M NaCl was added to yield a final concentration of 0.2 M. To this, 2 volumes of 100% ethanol were added and the sample was incubated 15 minutes at −80° C. The DNA was sedimented by centrifuging the sample at 16,000×g for 10 minutes at 4° C. After decanting the supernatant, the DNA was washed with 100% prechilled ethanol. The sediment was air dried and resuspended in sterile deionized water (20 μl) and stored at −20° C. Larger scale plasmid preparations were made using QIAGEN® Midi-plasmid Purification kit according to the manufacturer's protocol (QIAGEN Inc., Valencia, Calif.). The concentration of plasmid DNA was obtained by reading the absorbance of a 1:10 dilution of the preparation at 260 nm (GeneQuant II, Pharmacia Biotech).

Restriction Enzyme Digestion of Plasmid Constructs

The recombinant plasmids were subjected to restriction enzyme digestion as a means of screening for the presence of the desired construct. Digestions were performed by mixing 2 μl of enzyme specific buffer, 1 μl digestion enzyme (Promega, Madison, Wis.), 1 μl to 4 μl plasmid DNA preparation and sterile deionized water, up to a total volume of 20 μl. The digestions were incubated for 3 hours in a 37° C. waterbath. The nhaR construct was digested with HindIII (10 U/μl) and EcoRV (10 U/μl) enzymes. The leuO construct was digested with ApaI (12 U/μl )and EcoRV. The construct containing the yjjQ gene was digested with the enzymes Alw441 (10 U/μl) and EcoRV. The yojN construct was digested with BglI (10 U/μl) and EcoRV.

Transformation of Transposon Mutants or Transductants

Transposon mutants or transductants were transformed using the pCR2.1 plasmid constructs as previously described. A vector only control (pCR2.1 without an insert) was transformed into the chosen strains as a control. Selection of transformants was based on antibiotic resistance as previously described. Biofilm assays (previously described) were performed and the results compared to that of the parent strain.

Sequencing of Cloned Gene

The cloned gene in the recombinant plasmid designated pCRnhaR (which successfully complemented the corresponding transposon mutant) was sequenced to ensure the inserted gene was free of any PCR-generated mutations. This was accomplished by using a primer specific for a sequence upstream of the insertion site (M13UP) as well as several primers designed to anneal to sequences within the insert (nhaR1, nhaR2, nhaR3 and nhaR4). Sequences of these primers can be found on Table 2. Plasmid preparations were made and DNA concentrations determined as described previously. The samples were then sent to the University of Arizona for sequencing. The sequence information was compared to the E coli K12 databases at the NCBI internet web site for confirmation.

### Example 3

### Genes Altering Biofilm Formation

Transposon mutagenesis produced 800 random insertion mutants. Using microtiter assay to screen for altered biofilm formation ability, 120 of these mutants displayed an increase or decrease in biofilm production as compared to the parent. BLAST searches against Escherichia coli K12 genomic databases revealed 120 matches. Of this total, 61 were from mutant strains with a decrease in biofilm formation and 59 were from mutant strains with an increase in biofilm production (Tables 3 & 4). Many of the same genes were interrupted in more than one mutant. Multiple mutations were isolated from several operons as well. The genes from the ycd operon, for example, were interrupted in 22 mutants that exhibited decreased biofilm.

TABLE 3

| | | | |
|---|---|---|---|
| | | Mutation location | Biofilm Ratio |
| Gene/Function | Strain | Relative to inition codon | (Parent:Mutant) |

Mutant strains with a decrease in biofilm
formation compared to parent TRMG.

| | | | |
|---|---|---|---|
| aroD - amino acid biosynthesis | 87C5 | +259 | 1.5:1 |
| | 90E7 | +267 | 2:1 |
| b1904 - protein modulating ferritin | 11E4 | −79 | 2:1 |
| encoding gene ftn | | | |
| | 16C8 | −49 | 24:1 |
| | 31G6 | −78 | 2:1 |
| b2531 - protein modulating biofilm | 91F9 | +67 | 2:1 |
| formation | | | |
| clpX - serine protease chaperone | 63E2 | −10 | 2:1 |
| fucA - degradation of carbon | 24E12 | +43 | 8:1 |
| compounds; L-fuculose- | | | |
| 1-phosphate aldolase | | | |
| glnE - adenylyl transferase; | 68E11 | +2176 | 2:1 |
| activation/deactivation | | | |
| of glutamine synthetase | | | |
| hscA - heat shock protein; | 35C6 | +1426 | 6:1 |
| Hsp 70 family | | | |
| nhaR - regulation of cation | 96B10 | +432 | 11:1 |
| transport | | | |
| leuL - leader peptide; leucine | 109B4 | −113 | 2:1 |
| biosynthesis | | | |
| leuO - regulator, LysR-like; | 14B7 | +150 | 4:1 |
| leucine biosynthesis | | | |
| | 41B10 | +150 | 9:1 |
| | 41G10 | +150 | 10:1 |
| leuO - continued | 89E11 | +150 | 8:1 |
| | 102G9 | +150 | 5:1 |
| | 106B2 | +150 | 5:1 |
| | 126C5 | +150 | 7:1 |
| miaA - modification of tRNA; | 140B5 | +731 | 9:1 |
| aminoacyl tRNA | | | |
| synthetases | | | |
| moaC - enzyme; molybdopterin | 75E11 | +1335 | 2:1 |
| synthesis | | | |
| modA - molybdate uptake; peri- | 123F5 | +255 | 4:1 |
| plasmic molybdate | | | |
| binding protein | | | |
| modC - molybdate uptake; ATP- | 51A10 | +1136 | 2:1 |
| binding component of | | | |
| molybdate transport | 56C11 | +1136 | 5:1 |
| prfC - protein synthesis; release | 150G7 | +955 | 4:1 |
| factor - 3 | | | |
| ptrB - protein degradation; protease | 32A4 | −16 | 14:1 |
| rbsK - degradation of carbon | 125A7 | +361 | 2:1 |
| compounds; ribokinase | | | |
| rnpB - RNA degradation; Rnase P | 90A11 | +171 | 3:1 |
| trs5_7 - IS phage; transposase | 48A4 | +865 | 20:1 |
| wcaI - colanic acid biosynthesis; | 68A3 | +196 | >24:1 |
| glycosyl transferase | | | |
| ycdP - polysaccharide adhesin | 150E3 | +146 | 20:1 |
| synthesis | | | |
| ycdQ - protein product homologous | 1G3 | +672 | 16:1 |
| to IcaA in Staphylococcus | 46E5 | +204 | 10:1 |
| aureus; biosynthesis of | | | |
| polysaccharide adhesin | 86E7 | +471 | 10:1 |
| | 86F11 | +471 | 11:1 |
| ycdR - protein product homologous | 84A10 | +1461 | 11:1 |
| to IcaB in S. aureus; | | | |
| biosynthesis of polysaccharide | 93E3 | +1461 | 12:1 |
| adhesin | | | |
| | 115C12 | +1216 | 16:1 |
| | 155F4 | +510 | 20:1 |
| | 169G4 | +868 | 18:1 |
| ycdS - outer membrane protein | 12F12 | +672 | 23:1 |
| | 26G11 | +2064 | 8:1 |
| | 31A4 | +1124 | 7:1 |
| | 44C2 | +1124 | 10:1 |
| | 49C2 | +546 | 9:1 |
| | 49G12 | +672 | 9:1 |
| | 66E10 | +2884 | 12:1 |
| | 73E6 | +2055 | 16:1 |
| | 73E9 | +1987 | 12:1 |
| | 73F2 | +2061 | 12:1 |
| | 110G8 | +908 | 9:1 |
| | 141G4 | +956 | 18:1 |
| yecK - enzyme; | 62C9 | +479 | 20:1 |
| cytochrome-C type | | | |
| protein | 130E8 | −26 | 24:1 |
| yedK - protein - cellular | 37B5 | +649 | 11:1 |
| respiration | | | |
| yjcC - protein modulates | 27F9 | −54 | 2:1 |
| biofilm formation | | | |
| yjjQ - regulator modulates | 141G2 | +502 | 16:1 |
| biofilm formation | | | |
| | 160A8 | +506 | 5:1 |
| ykgK - regulator modulates | 145F10 | +344 | 3:1 |
| biofilm formation | | | |

Mutant strains with an increase in biofilm
formation compared to parent TRMG F/M.

| | | | |
|---|---|---|---|
| arcB - global regulatory function, | 136E3 | +710 | 1:7 |
| respiration; protein kinase/ | | | |
| phosphatase | | | |
| b1936 - protein; | 77A5 | +1057 | 1:11 |
| homologous to intG, | | | |
| a defective integrase gene | | | |
| in Escherichia coli 0157:H7 | | | |
| dnaKp2 promoter - Hsp chaperone; | 64F2 | +12 | 1:9 |
| DNA synthesis, and motility | | | |
| flgB - flagellar biosynthesis; | 115B7 | +117 | 1:7 |
| basal-body rod | | | |
| | 167C2 | +102 | 1:8 |
| flgE - flagellar biosynthesis; | 14C10 | +462 | 1:10 |
| hook Protein | | | |
| flgH - flagellar biosynthesis; | 98E11 | +11 | 1:5 |
| ring protein | | | |
| flgI - flagellar biosynthesis; product | 152B4 | +1062 | 1:9 |
| homologous to basal body | | | |
| P-ring in Salmonella | | | |
| flgK - flagellar biosynthesis; hook- | 111G8 | +152 | 1:4 |
| filament junction protein | | | |
| flhA - flagellar biosynthesis; | 126A3 | +907 | 1:7 |
| flagella regulon member | | | |
| | 126G2 | +907 | 1:7 |
| flhB - flagellar biosynthesis; | 61G2 | −20 | 1:10 |
| flagella regulon member | | | |
| flhD - flagellar biosynthesis; | 110F12 | −95 | 1:9 |
| transcription initiation factor | | | |
| fliA - flagellar biosynthesis; | 78E3 | +594 | 1:7 |
| Alternate sigma factor | | | |
| fliD - flagellar biosynthesis; | 1B10 | +27 | 1:10 |
| filament capping protein | | | |
| | 115A3 | +494 | 1:2 |
| | 163E7 | +27 | 1:4 |
| | 163F3 | +27 | 1:8 |
| fliF, predicted promoter - flagellar | 85G11 | −5 | 1:6 |
| biosynthesis; basal body ring | | | |
| fliG - flagellar biosynthesis; | 43B10 | +664 | 1:11 |
| motor component | | | |
| | 89A8 | +802 | 1:6 |
| fliI - flagellar biosynthesis; | 67E10 | +747 | 1:6 |
| flagellum ATP synthase | | | |
| | 70G12 | +1349 | 1:5 |
| fliL - flagellar biosynthesis; | 122F6 | +152 | 1:4 |
| surface structure | | | |
| fliM - flagellar biosynthesis; | 13G10 | +204 | 1:11 |
| motor component | | | |
| | 38G7 | +488 | 1:11 |
| | 51B12 | +535 | 1:6 |
| | 66F4 | +489 | 1:10 |
| | 67C8 | +481 | 1:6 |
| | 67C9 | +481 | 1:8 |
| | 76F11 | +484 | 1:5 |
| | 132B8 | +176 | 1:7 |
| fliP - flagellar biosynthesis; | 42G6 | +340 | 1:12 |
| flagella regulon member | | | |
| | 49G9 | +388 | 1:12 |
| | 57E7 | +395 | 1:7 |
| | 103C8 | +397 | 1:9 |
| | 110E8 | +390 | 1:6 |
| | 125C9 | +397 | 1:7 |
| fliR - flagellar biosynthesis; | 98E7 | +585 | 1:6 |
| flagella regulon member | | | |
| | 123E4 | +130 | 1:6 |
| frdA - enzyme; anaerobic | 45C8 | +67 | 1:4 |
| respiration, fumarate | | | |
| reductase | | | |
| mreB - cell division; cell shape, | 104G4 | −121 | 1:2 |
| regulator of peptidoglycan | | | |
| synthetase | | | |
| mdoG - enzyme; periplasmic | 98G12 | +142 | 1:5 |
| oligosaccharide synthesis | | | |
| rep - enzyme; helicase, | 39C9 | +270 | 1:3 |
| DNA-dependent ATPase | | | |
| rfaG - enzyme; LPS biosynthesis, | 12E12 | +163 | 1:7 |
| glucosyltransferase I | | | |
| | 74B5 | +62 | 1:2 |
| rfaP - enzyme; LPS biosynthesis, | 139G5 | +496 | 1:5 |
| hepatose phosphorylation | | | |
| rfaQ - enzyme; LPS core | 63A9 | +380 | 1:4 |
| biosynthesis | | | |
| | 71A4 | −44 | 1:2 |
| | 94G6 | −44 | 1:4 |
| | 107B12 | −65 | 1:2 |
| | 123B8 | +361 | 1:3 |
| | 125F2 | +104 | 1:3 |
| rnhB - enzyme; degradation of | 92G7 | +500 | 1:3 |
| RNA, RNAseHII | | | |
| | 92G9 | +500 | 1:3 |
| tolA - periplasmic protein; outer | 78F6 | +1189 | 1:2 |
| membrane integrity | | | |
| wecB - enzyme; UDP-N-acetyl | 43F5 | +1112 | 1:2 |
| glucosamine-2-epimerase, | | | |
| sugar nucleotide biosynthesis | | | |
| yojN - regulator; part of | 103E8 | +2147 | 1:7 |
| a phosphorelay signaling | | | |
| pathway; affects swarming | | | |
| behaviour | | | |
| yhjH - protein modulates | 36E2 | +678 | 1:3 |
| biofilm formation | | | |

References
Alavi, Mohammad and Belas, R. “Surface Sensing, Swarmer Cell Differentiation, and Biofilm Development” Methods in Enzymology Vol 336, p. 29-40, 2001
Altschul, Stephen F, Gish, W., Miller, W., Myers, E. W. and Lipman, D. “Basic Local Alignment Search Tool” Journal of Molecular Biology p. 403-410, 1990
Anderson, Lisa A., McNairn, E., Leubke, T., Pau, R., Boxer, D. “ModE-Dependent Regulation of the Molybdenum Cofactor Operon MO in Escherichia coli” Journal of Bacteriology p. 7035-7043, December 2000
Balaban, N., Gov Y., Bitler, A., Boelaert, J. R. “Prevention of Staphylococcus aureus biofilm on dialysis catheters and adherence to human cells”, Kidney Int. Vol 63 p. 340-345, January 2003
Barua, Soumitra, Yamashino, T., Hasegawa, T., Yokoyama, K., Torii, K., Ohta, M. “Involvement of surface polysaccharides in the organic acid resistance of Shiga toxin-producing Escherichia coli 0157:H7” Molecular Microbiology Vol. 43, p. 629-640, 2002
Belas, Robert, Schneider, R., Melch, M., “Characterization of Proteus mirabilis Precocious Swarming Mutants: Identification of rsbA, Encoding a Regulator of Swarming Behavior” Journal of Bacteriology p. 6126-6139, December 1998
Costerton, J. W., Stewart, S. S. and Greenberg, E. P. “Bacterial Biofilms: A Common Cause of Persistent Infections” Science Vol 284 p. 1318-1322, May 21, 1999
Costerton, J. W. and Lewandowski, Z. “Microbial Biofilms” Annual Review Microbiology p. 711-745, 1995
Costerton, J. W., Lewandowski, Z., DeBeer, D., Caldwell, D., Korber, D. and James, G. “Biofilms, the Customized Microniche” Journal of Bacteriology p. 2675-2679, April 1994
Costerton, J. W., Cheng, K. J., Geesey, G. G., Ladd, T. I., Nickel, J. C., Dasgupta, M and Marrie, T. J. “Bacterial Biofilms in Nature and Disease” Annual Review Microbiology p. 435-464, 1987
Cramton, Sarah E., Gerke, C., Schnell, N., Nichols, W., Gotz, F. “The Intercellular Adhesion (ica) Locus Is Present in Staphylococcus aureus and is Required for Biofilm Formation” Infection and Immunity p. 5427-5433, October 1999
Danese, Paul N., Pratt, L. A. and Kolter, R. “Biofilm Formation as a Developmental Process” Methods In Enzymology Vol 336 p. 19-26, 2001
Davey, Mary Ellen and O'Toole, G. A. “Microbial Biofilms: from Ecology to Molecular Genetics” Microbiology and Molecular Biology Reviews p. 847-867, December 2000
Dover, Nir and Padan, E., “Transcription of nhaA, the Main Na/H⁺ Antiporter of Escherichia coli, Is Regulated by Na⁺ and Growth Phase” Journal of Bacteriology p. 644-653, Janurary 2001
Genevaux, Pierre, Baudia, P., Dubow, M. S. and Oudega, B. “Identification of Tn10 Insertions in the dsbA gene affecting Escherichia coli biofilm formation” FEMS Microbiology Letters p. 403-409, 1999
Genevaux, Pierrre, Muller, S., and Bauda, P. “A rapid screening procedure to identify mini-Tn10 insertion mutants of Escherichia coli K-12 with altered adhesion properties” FEMS Microbiology Letters p. 27-30, 1996
Gon, Stephanie, Patte, J., Mejean, V., Iobbi-nivol, C. “The torYZ (yecK bisZ) Operon Encodes a Third Respiratory Trimethylamine N-Oxide Reductase In Escherichia coli” Journal of Bacteriology p. 5779-5786, October 2000
Huber, Birgit, Riedal, K., Kothe, M., Givskov, M., Molin, S. and Eberl, L. “Genetic Analysis of functions involved in the late stages of biofilm development in Burkholderia cepacia H111” Molecular Microbiology p. 411-426, 2002
Jackson, Debra W., Suzuki, K., Oakford, L., Simecka, J. W., Hart, M. E. and Romeo, T. “Biofilm Formation and Dispersal under the Influence of the Global Regulator CsrA of Esherichia coli” Journal of Bacteriology p. 290-301, January 2002
Kennedy, E. P. “Membrane-derived oligocaccharide (periplasmic beta-D-glucans) of Escherichia coli osmoadaptation, recognition of eukaryotic hosts, biosynthesis” In Escherichia coli and Salmonella: Cellular and Molecular
Biology, Neidhardt, F. C., Curtiss, III R., Ingraham, J. L., Lin, E. C. C., Low, K. B., Magasanik, B., et. al., Vol 1, 2^{nd} ed., p. 1064-1072 ASM Press, 1996
Kleckner, N., Bender, J., and Gottesman, S. “Uses of Transposons with Emphasis on Tn10” Methods in Enzymology, Vol. 204, p. 139-179, Academic Press, 1991
Kolter, Roberto and Losick, R. “One for All and All for One” Science Vol 280, p. 226-227, Apr. 10, 1998
Kwon, Ohsuk, Georgellis, D., Lynch, A., Boyd, D., Lin, E. “The ArcB Sensor Kinase of Escherichia coli: Genetic Exploration of the Transmembrane Region” Journal of Bacteriology, p. 2960-2966, May 2000
Lamont, Richard J. and Bryers, James D. “Biofilm-Induced Gene Expression and GeneTransfer” Methods in Enzymology, Vol. 336, p. 84-94, Academic Press, 2001
Landini, Paolo, Zehnder, A. “The Global Regulatory hns Gene Negatively Affects Adhesion to Solid Surfaces by Anaerobically Grown Escherichia coli by Modulating Expression of Flagellar Genes and Lipopolysaccharide Production” Journal of Bacteriology p. 1522-1529, March, 2002
Lewis, Kim “Riddle of Biofilm Resistance” Antimicrobial Agents and Chemotherapiy p. 999-1007, April 2001
Liu, M. Y., G. Gui, B. Wei, J. F. Preston III, L. Oakford, U. Yuksel, D. P. Giedroc, and T. Romeo. 1997. The RNA molecule CsrB binds to the global regulatory protein CsrA and antagonizes its activity in Escherichia coli J. Biol. Chem. 272:17502-17510.
Matsushika, Akinori, Mizuno, T. “A Dual-Signaling Mechanism Mediated by the ArcB Hybird Sensor Kinase Containing the Histidine-Containing Phosphotransfer Domain in Escherichia coli” Journal of Bacteriology p. 3973-3977, August 1998
Miller, J. H. Experiments in Molecular Genetics, Cold Spring Harbor Laboratory, Cold Spring Harbor, N.Y., 1972
Mireles, Joe Robert, Toguchi, A., Harshey, R. “Salmonella enterica Serovar Typhimurium Swarming Mutants with Altered Biofilm-Forming Abilities: Surfactin Inhibits Biofilm Formation” Journal of Bacteriology, p. 5848-5854, October 2001
O'Toole, George A. and Kolter, R. “Initiation of biofilm formation in Pseudomonos Flourescens WCS365 proceeds via multiple, convergent signalling pathways: A Genetic analysis” Molecular Microbiology p. 449-461, 1998
Otto, Karen, Norbeck, J., Larsson, T., Karlsson, K., Hermansson, M. “Adhesion of Type I-Fimbriated Escherichia coli to Abiotic Surfaces Leads to Altered Composition of Outer Membrane Proteins” Journal of Bacteriology p. 2445-2453, April 2001
Otto, Karen and Silhavy, T. J. “Surface sensing and adhesion of Escherichia coli Controlled by the CPX-signaling pathway” PNAS Vol. 99, p. 2287-2292, February 2002
Page, Frederic, Altabe, S., Hugouvieux-Cotte-Pattat, N., Lacroix, J., Robert-Baudouy, J., Bohin, J. “Osmoregulated Periplasmic Glucan Synthesis Is Required for Erwinia chrysanthemi Pathogenicity” Journal of Bacteriology p. 3134-3141, May 2001
Palmer, Robert J. and White, D. C. “Developmental biology of biofilms: implications for Treatment and control” Trends in Microbiology Vol 5, No. 11, p. 435-440, November 1997
Potera, Carol “Forging a Link Between Biofilms and Disease” Science Vol 283 p. 1837-1839, Mar. 19, 1999
Pratt, Leslie and Kolter, R. “Genetic analysis of Esherichia coli biofilm formation: Roles of flagella, motility, chemotaxis and type I pili” Molecular Microbiology Vol. 30, p. 285-293, 1998
Prigent-Combaret, Claire, Prensier, G., Thi, T. T. L., Vidal, O., Lejeune, P., and Dorel, C. “Developmental pathway for biofilm formation in curli-producing Escherichia coli strains: role of flagella, curli and colonic acid” Environmental Microbiology Vol. 2, p. 450-464, 2000
Prigent-Combaret, Claire, Vidal, O., Dorel, C. and Lejeune, P. “Abiotic Surface Sensing and Biofilm-Dependent Regulation of Gene Expression in Escherichia coli” Journal of Bacteriology p. 5993-6002, October 1999
Romeo, Tony “Global regulation by the small RNA-binding protein CsrA and the non-Coding RNA molecule CsrB” Molecular Microbiology p. 1321-1330, 1998
Romeo, T., J. Black, and J. Preiss. 1990. Genetic regulation of glycogen biosynthesis in Escherichia coli: in vivo effects of the catabolite repression and stringent response systems in glg gene expression. Curr. Microbiol. 21:131-137.
Romeo, T., M. Gong, M. Y. Liu, and A.-M. Brun-Zinkernagel. 1993. Identification and molecular characterization of csrA, a pleiotropic gene from Escherichia coli that affects glycogen biosynthesis, gluconeogenesis, cell size, and surface properties. J. Bacteriol. 175:4744-4755.
Sambrook, Joseph, Russell, David W. Molecular cloning A Laboratory Manual 3^{rd} Ed., Cold Spring Harbor Laboratory press, Cold Spring Harbor, N.Y., p. 8.21-8.24, 2001
Sauer, Karin, Camper, A., “Characterization of Phenotypic Changes in Pseudomonas Putida in Response to Surface-Associated Growth” Journal of Bacteriology, p. 6579-6589, November 2001
Sauer, Karin, Camper, A., Ehrlich, G., Costerton, J., Davies, D., “Pseudomonas aeruginosa Displays Multiple Phenotypes during Development as a Biofilm” Journal of Bacteriology p. 1140-1154, February 2002
Shi, Wenyuan, Zhou, Y., Wild, J., Adler, J., Gross, C., “DnaK, DnaJ, and GrpE Are Required for Flagellum Synthesis in Escherichia coli” Journal of Bacteriology p. 6256-6263, October 1992
Stanley, Nicola, Britton, R., Grossman, A. “Identification of Catabolite Repression as a Physiological Regulator of Biofilm Formation by Bacillus subtilis by use ofDNA Microarrays” Journal of Bacteriology p. 1951-1957, March 2003
Takeda, Shin-Ichiro, Fujisawa, Y., Matsubara, M., Aiba, H., Mizuno, T. “A novel feature of the multistep phosphorelay in Escherichia coli: a revised model of the RcsC→YojN→RcsB signalling pathway implicated in capsular synthesisand swarming behaviour” Molecular Microbiology Vol. 40(2), p. 440-450, 2001
Toesca, Isabelle, Perard, C., Bouvier, J., Gutierrez, C., Conter, A. “The transcriptional Activator NhaR is responsible for the osmotic induction of osmCₚₗ, a promoter of the stress-inducible gene osmC in Escherichia coli” Microbiology, Vol. 147, p. 2795-2803, 2001
Wei, Bangdong, Brun-Zinkemagel, A., Simecka, J., PruB, B., Babitzke, P., Romeo, T., “Positive regulation of motility and flhDC expression by the RNA-binding protein CsrA of Escherichia coli” Molecular Microbiology Vol. 40(1), p. 245-256, 2001
Watnick, Paula and Kolter, R. “Biolfilm, City of Microbes” Journal of Bacteriology p. 2675-2679, May 2000
Whiteley, M., Bangera, M., Bumgarner, R., Parsek, M., Teitzel, G., Lory, S., Greenberg, E. P. “Gene expression in Pseudomonas Biofilms” Nature Vol. 413 p. 860-864, 2001
Yang, Honghui, Liu, M., Romeo, T., “Coordinate Genetic Regulation of Glycogen Catabolism and Biosynthesis in Escherichia coli via the CsrA Gene Product” Journal of Bacteriology p. 1012-1017, February 1996

1. A method for identifying a compound which modulates biofilm formation, comprising identifying a compound which modulates the activity of a polynucleotide, or a polypeptide encoded by said polynucleotide or a control region of said polynucleotide, wherein the polynucleotide is a coding sequence selected from the group consisting of flgB, flgE, flgH, flgI, flgK, flhA, flhB, flhD, fliA, fliD, fliF, fliG, fliI, fliL, fliM, flip, fliR, dnaK, mdoG, rfaQ, rfaG, rfaP, wecB, yojN, arcB and frdA, b1936, yhjH, tolA, rnhB, rep, mreB, aroD, clpX, leuO, leuL, miaA, prfC, ptrB, rnpB, b2531, yedK, yjcC, yjjQ, ykgK, hscA and trs5_{—}7, fucA, rbsK, glnE, wcaI, modA, modC, MO, nhaA, yecK, ycdS, ycdR, ycdQ ycdP, and b1904, comprising the steps of:
(a) selecting a host cell having said polynucleotide;
(b) cloning said host cell and separating said clones into a test group and a control group;
(c) treating said test group using a compound; and
(d) determining the relative quantity of an expression product of said polynucleotide, as between said test group and said control group.

2. The method of claim 1, wherein said host cell is heterologous to said polynucleotide.

3. The method of claim 1, wherein said coding sequence is selected from the group consisting of nhaR, ptrB, yjjQ, wcaI, yecK, and b1904.

4. The method of claim 3, wherein said coding sequence is nhaR.

5. A method for screening a medium for a biofilm polypeptide encoded by a polynucleotide selected from the group consisting of flgB, flgE, flgH, flgI, flgK, flhA, flhB, flhD, fliA, fliD, fliF, fliG, fliI, fliL, fliM, flip, fliR, dnaK, mdoG, rfaQ, rfaG, rfaP, wecB, yojN, arcB and frdA, b1936, yhjH, tolA, rnhB, rep, mreB, aroD, clpX, leuO, leuL, miaA, prfC, ptrB, rnpB, b2531, yedK, yjcC, yjjQ, ykgK, hscA and trs5_{—}7, fucA, rbsK, glnE, wcaI, modA, modC, MO, nhaA, yecK, ycdS, ycdR, ycdQ ycdP, and b1904, comprising;
(a) labelling an antibody immunoreactive with said polypeptide with a marker molecule to form a conjugate;
(b) exposing said conjugate to said medium; and
(c) determining whether there is binding between said conjugate and a biomolecule in said medium, wherein said binding indicates the presence of said polypeptide.

6. The method of claim 5, wherein said polynucleotide is selected from the group consisting of nhaR, ptrb, yjjQ, wcaI, yecK, and b1904.

7. The method of claim 6, wherein said polynucleotide is nhaR.

8. A method for identifying a compound which modulates biofilm formation, comprising identifying a compound which modulates the activity of a polynucleotide, wherein the polynucleotide is a coding sequence selected from the group consisting of flgB, flgE, flgH, flgI, flgK, flhA, flhB, flhD, fliA, fliD, fliF, fliG, fliI, fliL, fliM, flip, fliR, dnaK, mdoG, rfaQ, rfaG, rfaP, wecB, yojN, arcB and frdA, b1936, yhjH, tolA, rnhB, rep, mreB, aroD, clpX, leuO, leuL, miaA, prfC, ptrB, rnpB, b2531, yedK, yjcC, yjjQ, ykgK, hscA and trs5_{—}7, fucA, rbsK, glnE, wcaI, modA, modC, MO, nhaA, yecK, ycdS, ycdR, ycdQ ycdP, and b1904, comprising the steps of:
(a) selecting a regulatory element from the 5′region of said polynucleotide;
(b) fusing said regulatory element to a reporter gene to create a fusion gene;
(c) selecting a host cell comprising said fusion gene;
(d) cloning said host cell and separating said clones into a test group and a control group;
(e) treating said test group using a compound; and
(f) determining the relative quantity of an expression product of said reporter gene, as between said test group and said control group.

9. The method of claim 8, wherein said polynucleotide is selected from the group consisting of nhaR, ptrB, yjjQ, wcaI, yecK, and b1904.

10. The method of claim 9, wherein said polynucleotide is nhaR.

11. A method for identifying an environmental factor which modulates biofilm formation, comprising identifying an environmental factor which modulates the activity of a polynucleotide, wherein the polynucleotide is a coding sequence selected from the group consisting of flgB, flgE, flgH, flgI, flgK, flhA, flhB, flhD, fliA, fliD, fliF, fliG, fliI, fliL, fliM, flip, fliR, dnaK, mdoG, rfaQ, rfaG, rfaP, wecB, yojN, arcB and frdA, b1936, yhjH, tolA, rnhB, rep, mreB, aroD, clpx, leuO, leuL, miaA, prfC, ptrB, rnpB, b2531, yedK, yjcC, yjjQ, ykgK, hscA and trs5_{—}7, fucA, rbsK, glnE, wcaI, modA, modC, MO, nhaA, yecK, ycdS, ycdR, ycdQ ycdP, and b1904, comprising the steps of:
(a) selecting a regulatory element from the 5′region of said polynucleotide;
(b) fusing said regulatory element to a reporter gene to create a fusion gene;
(c) selecting a host cell comprising said fusion gene;
(d) cloning said host cell and separating said clones into a test group and a control group;
(e) subjecting said test group with a different environmental condition as compared to said control group; and
(f) determining the relative quantity of an expression product of said reporter gene, as between said test group and said control group.

12. The method of claim 11, wherein said polynucleotide is selected from the group consisting of nhaR, ptrB, yjjQ, wcaI, yecK, and b1904.

13. The method of claim 12, wherein said polynucleotide is nhaR.

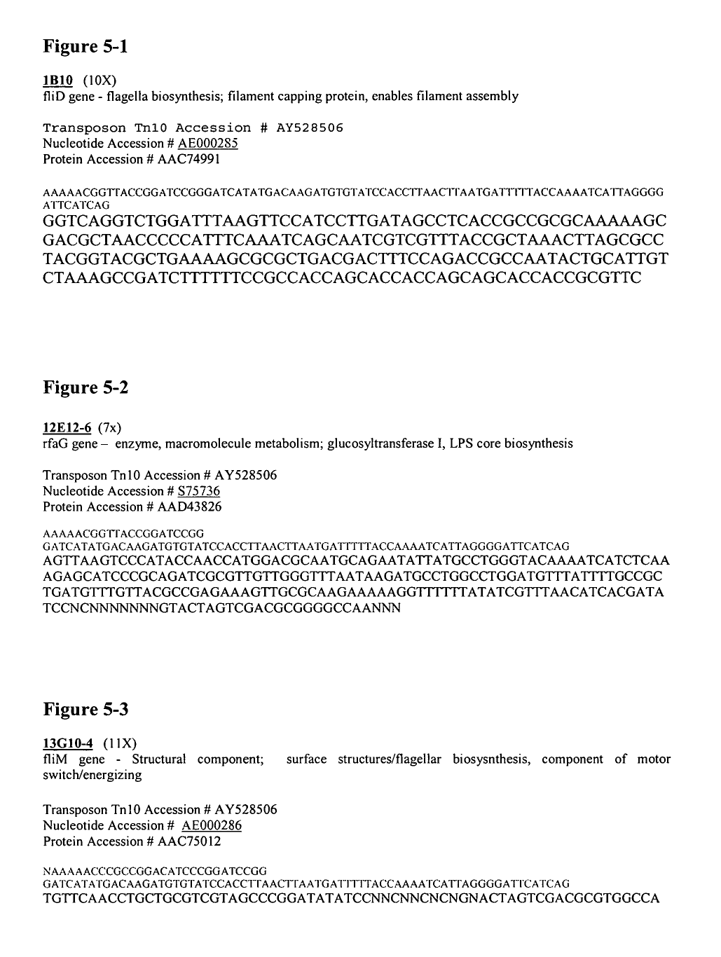

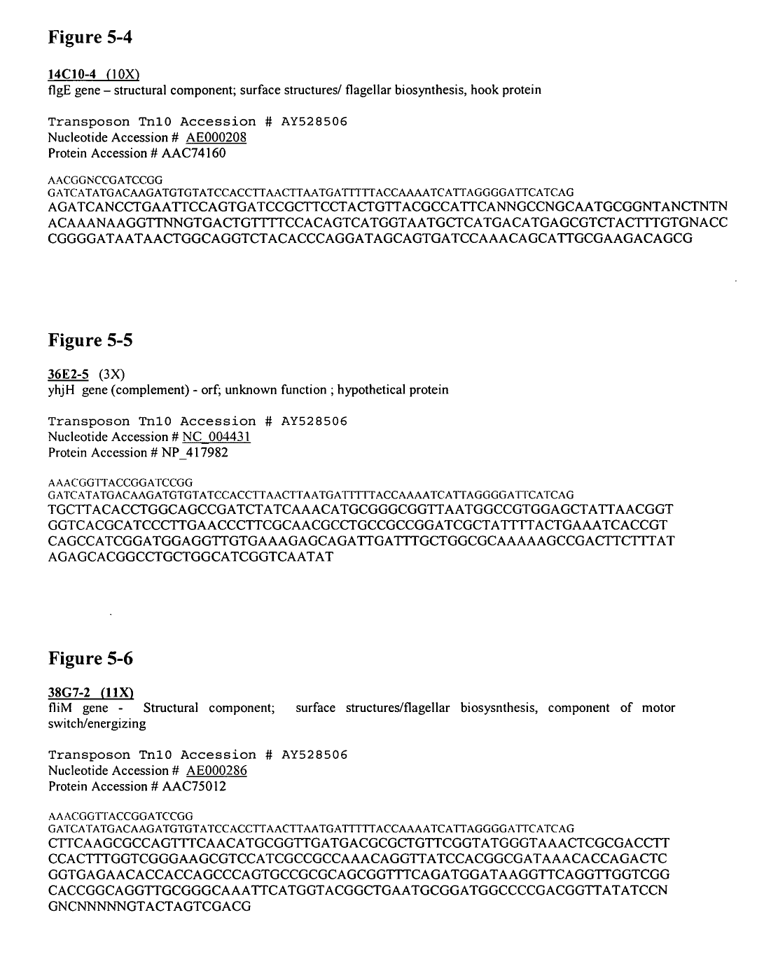

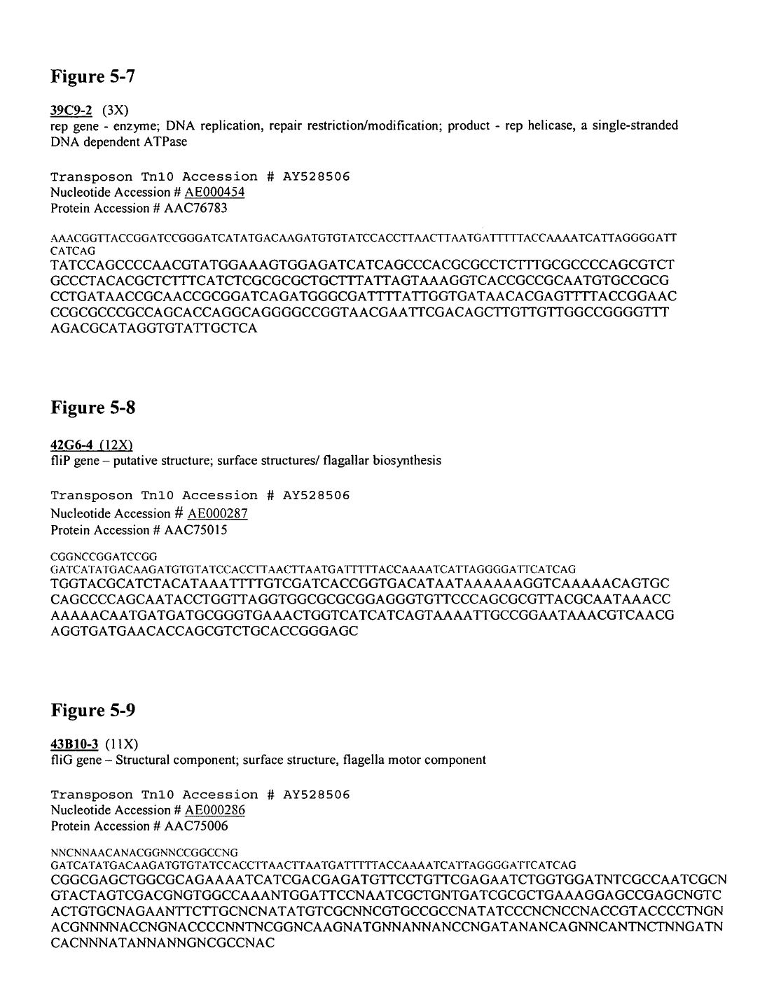

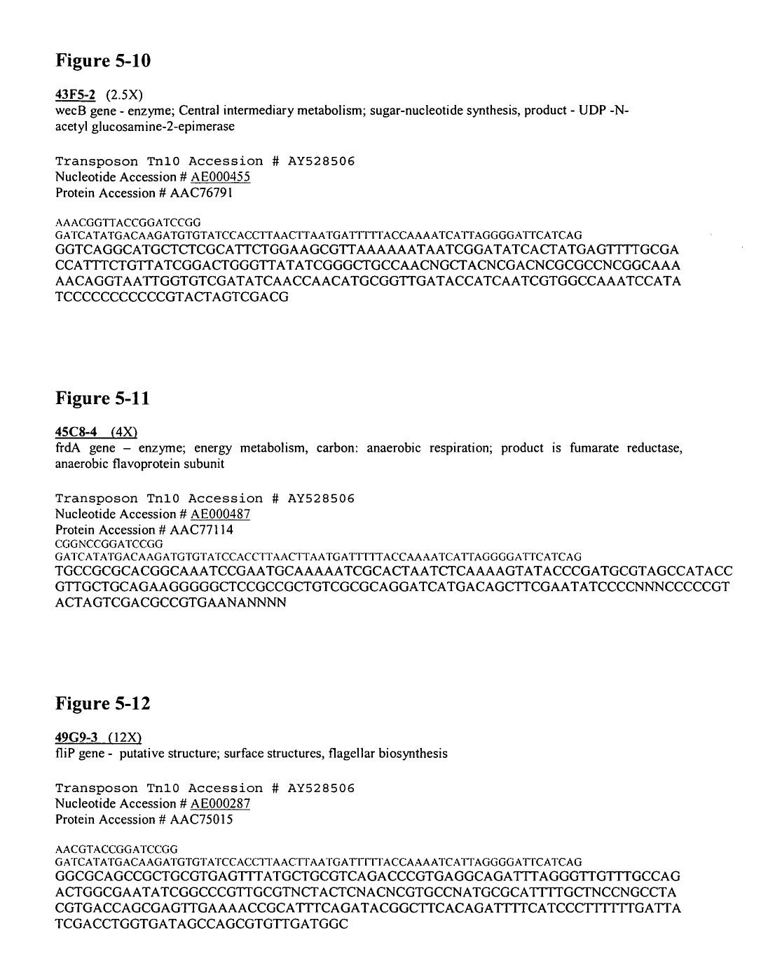

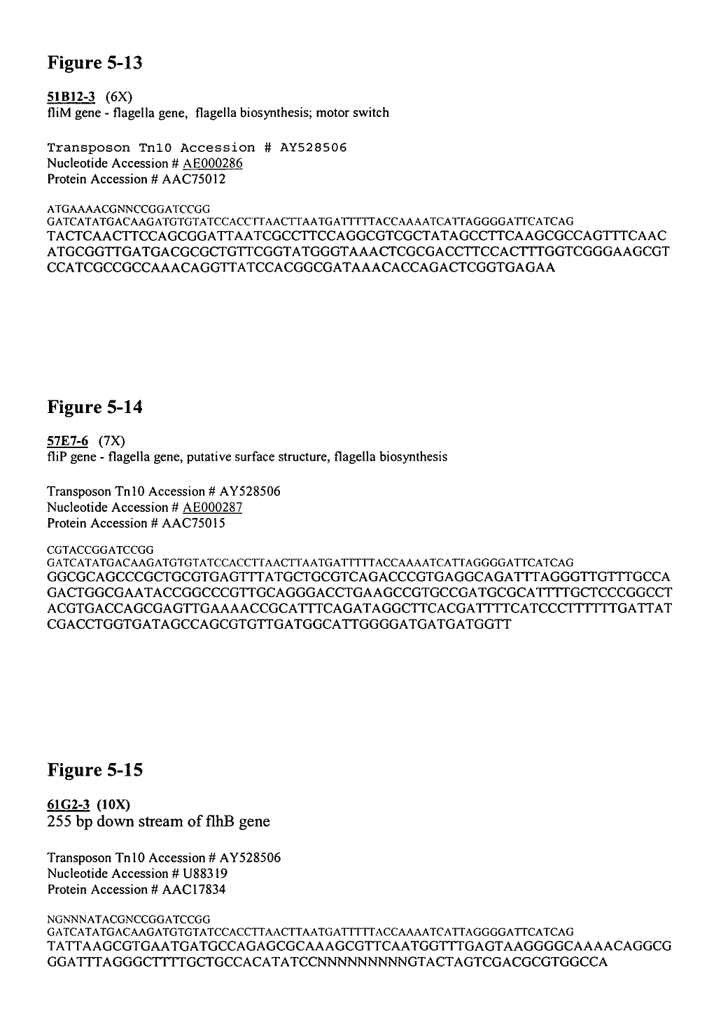

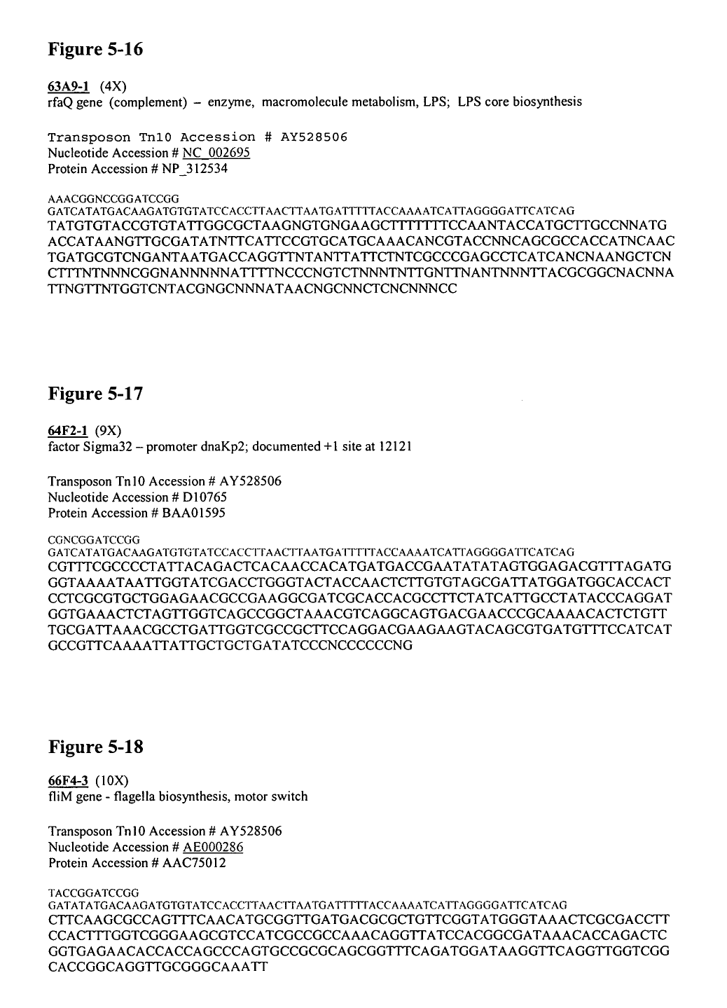

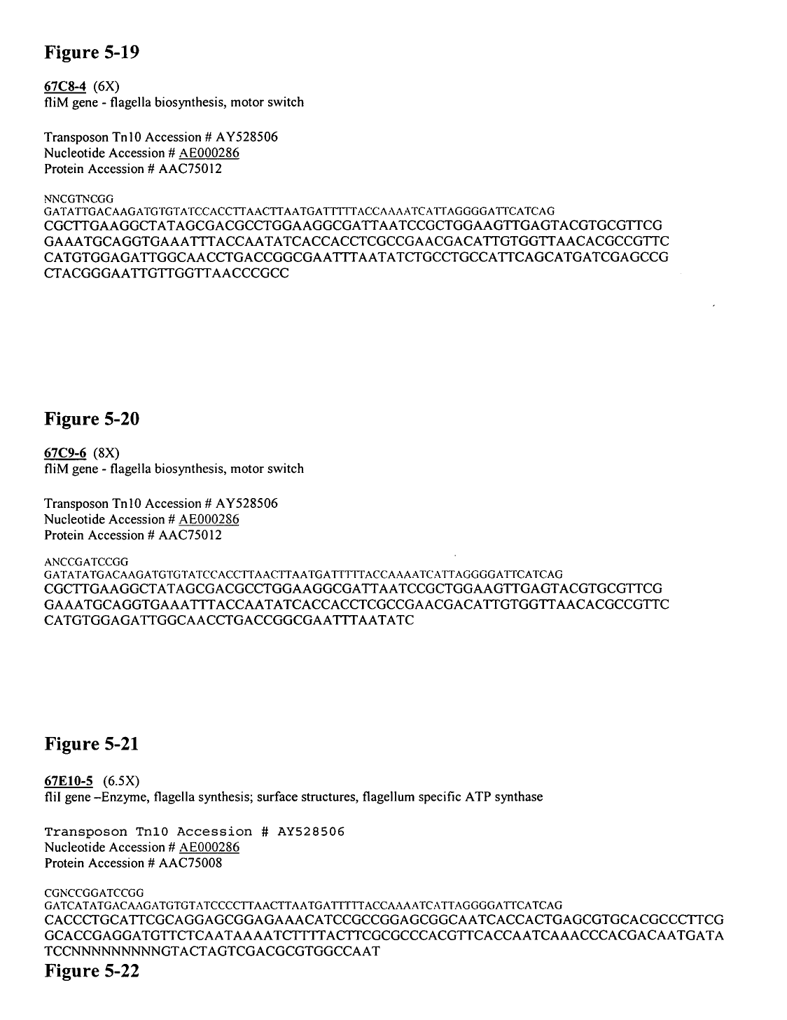

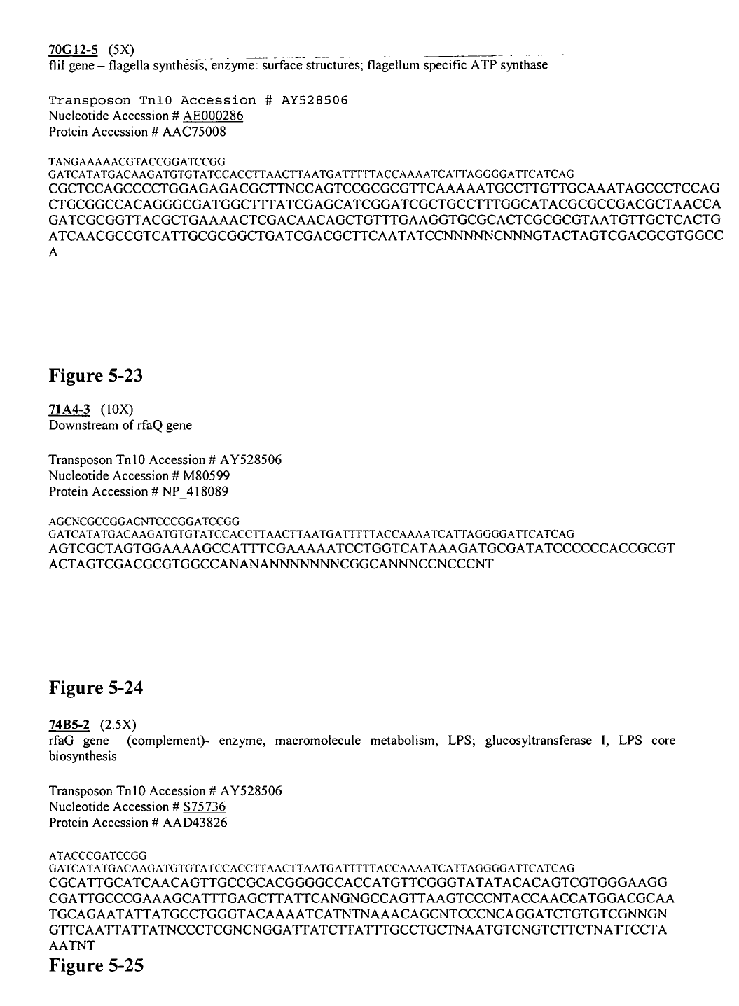

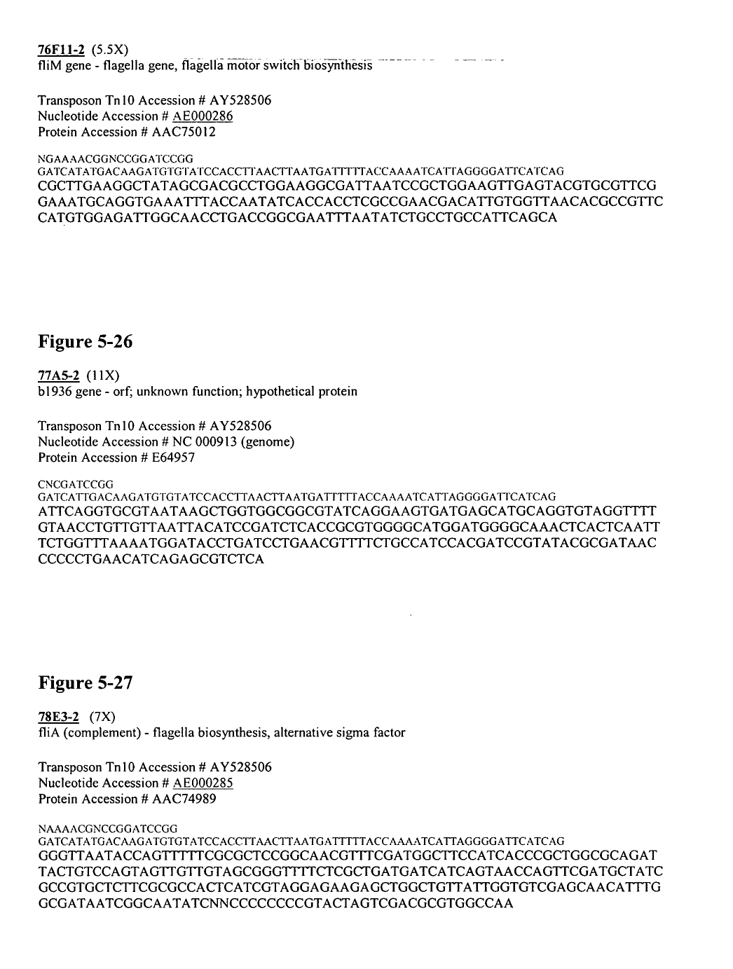

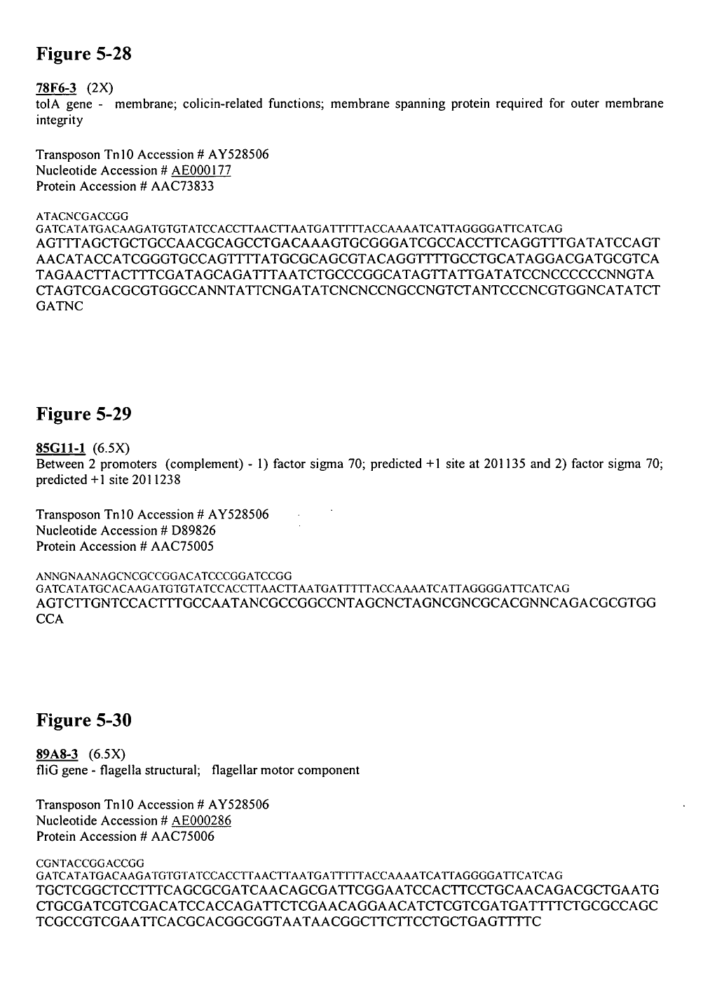

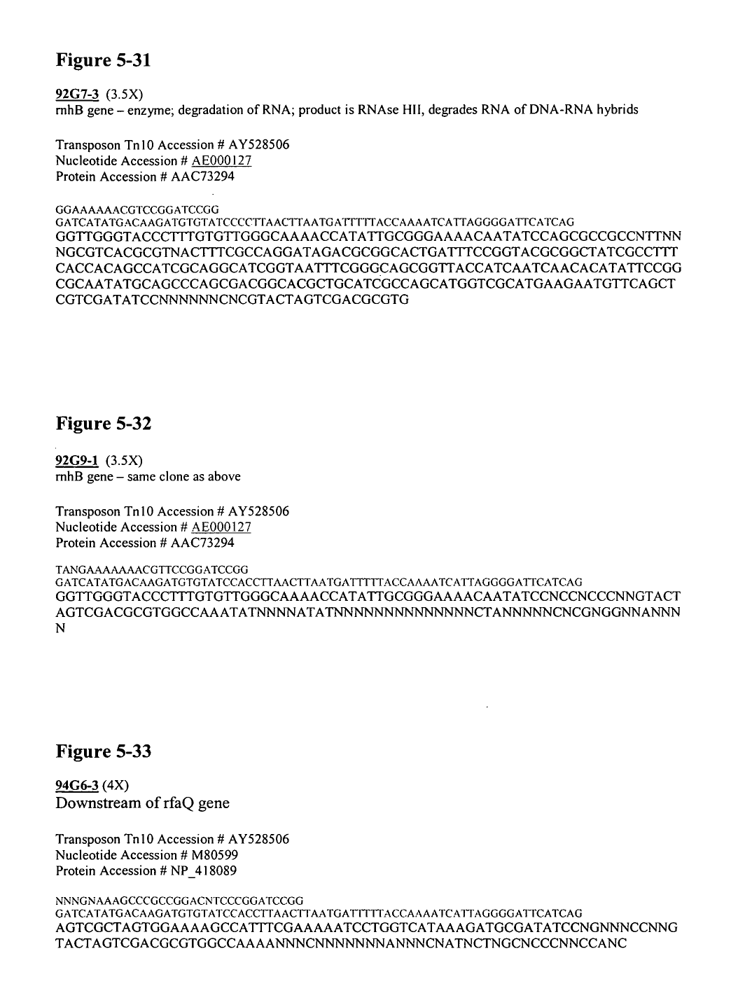

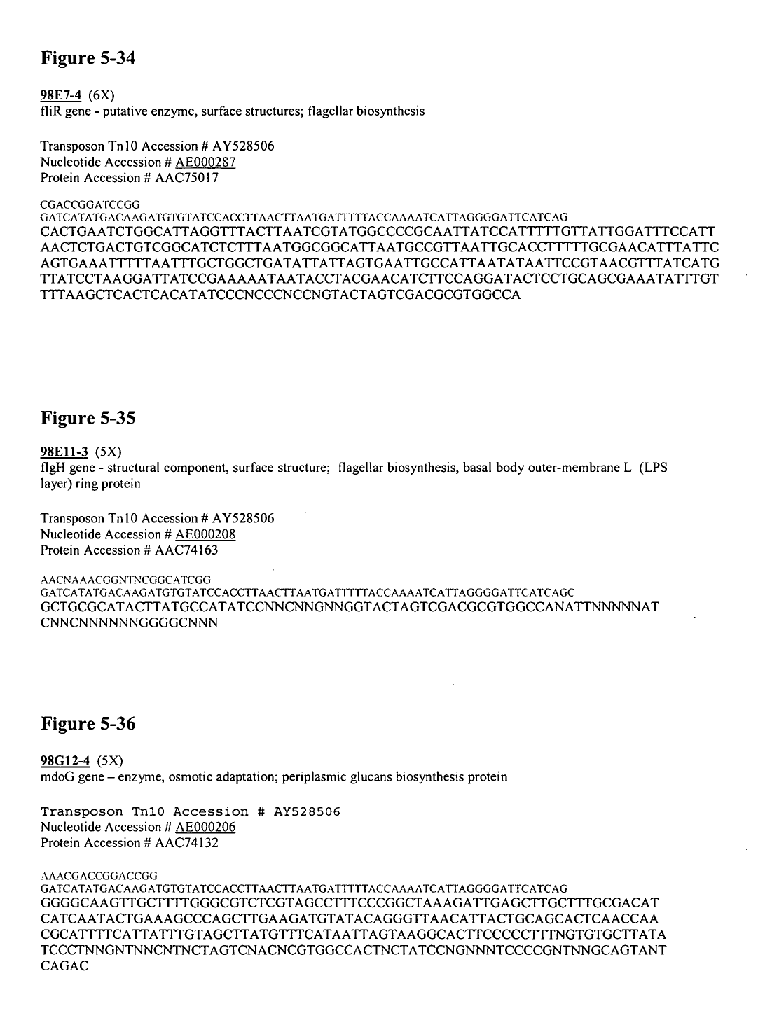

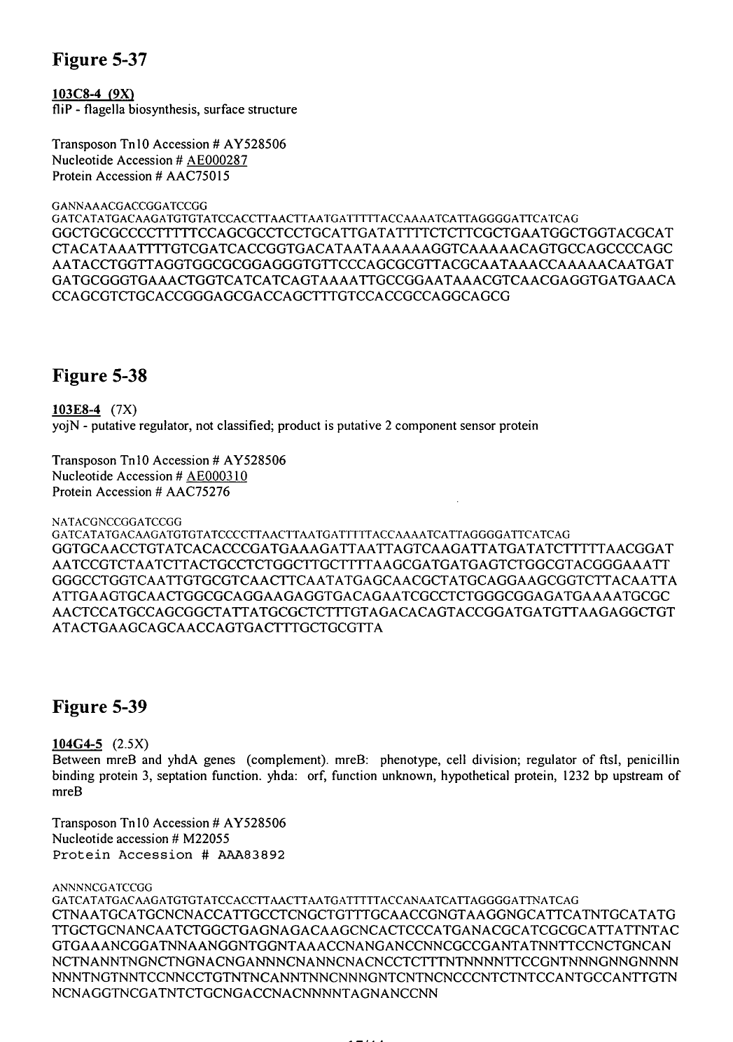

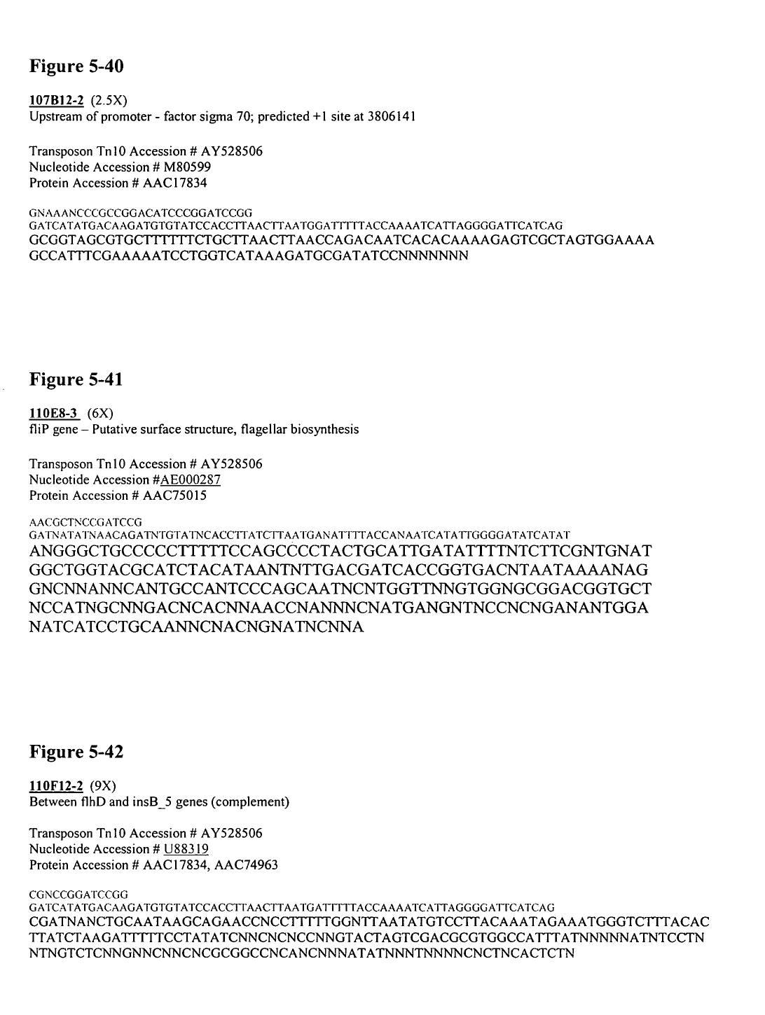

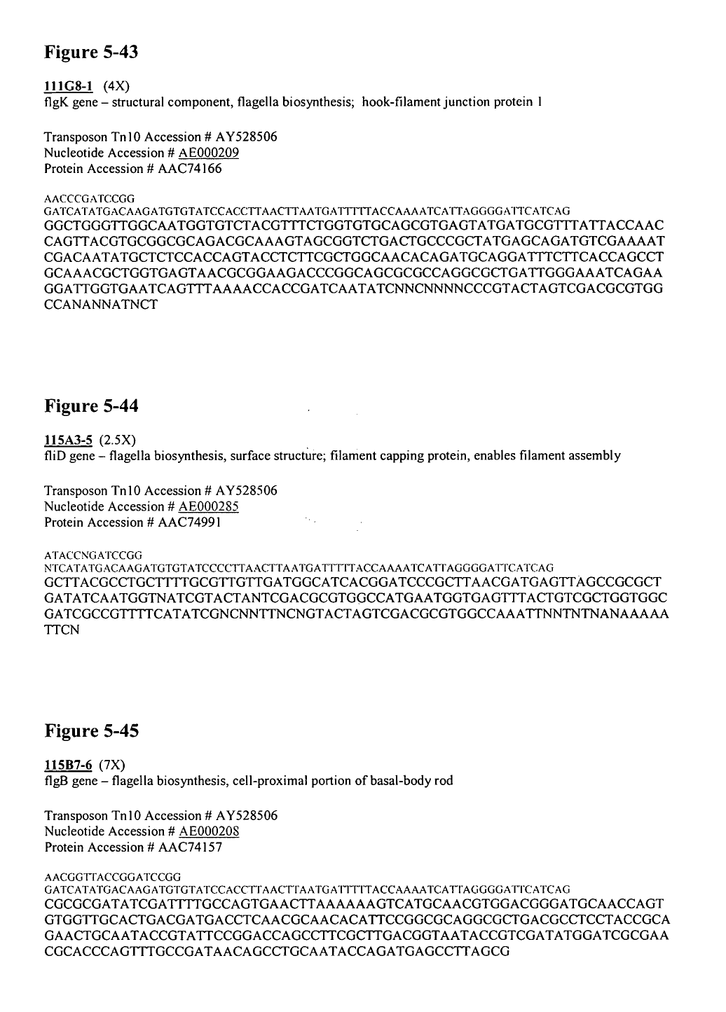

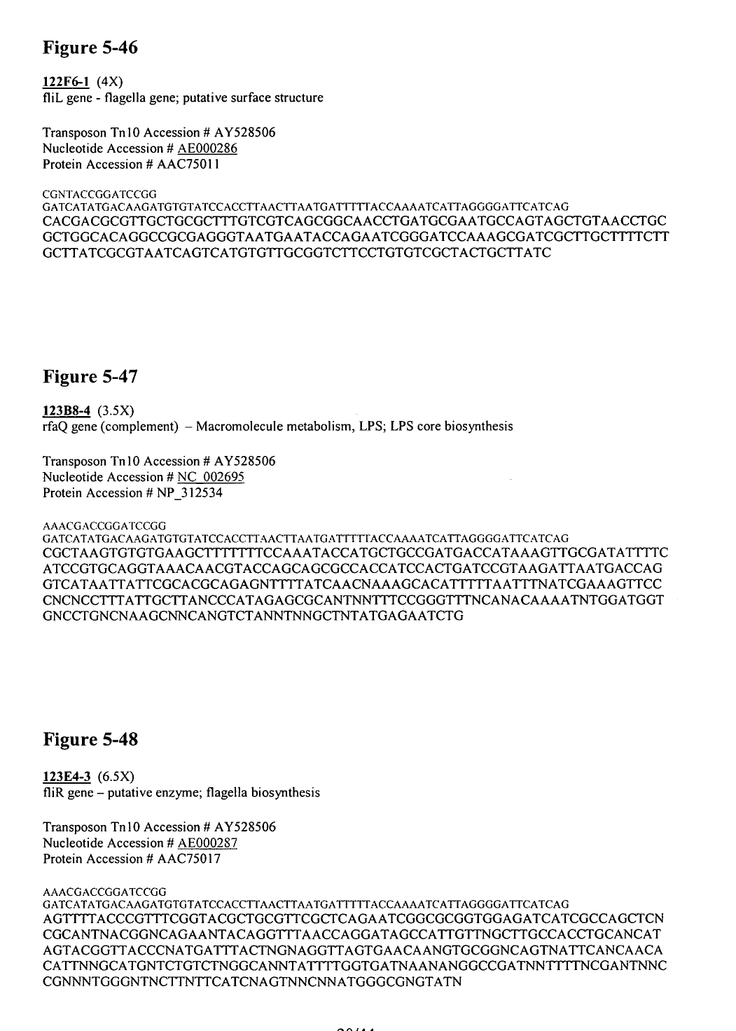

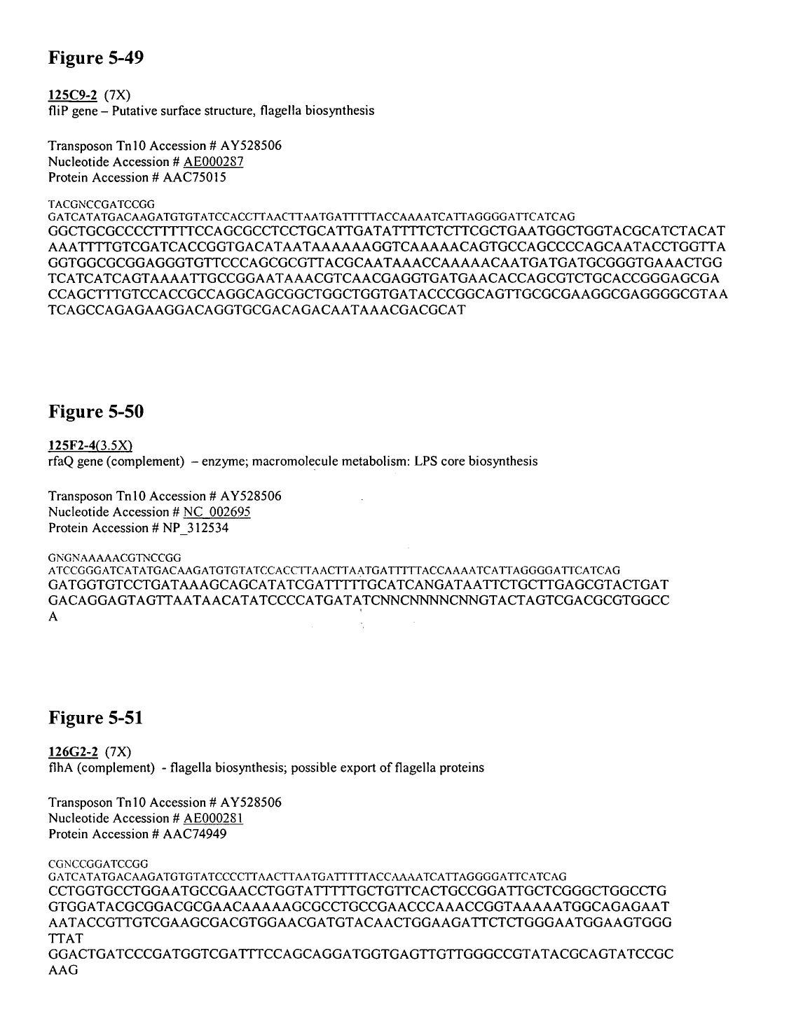

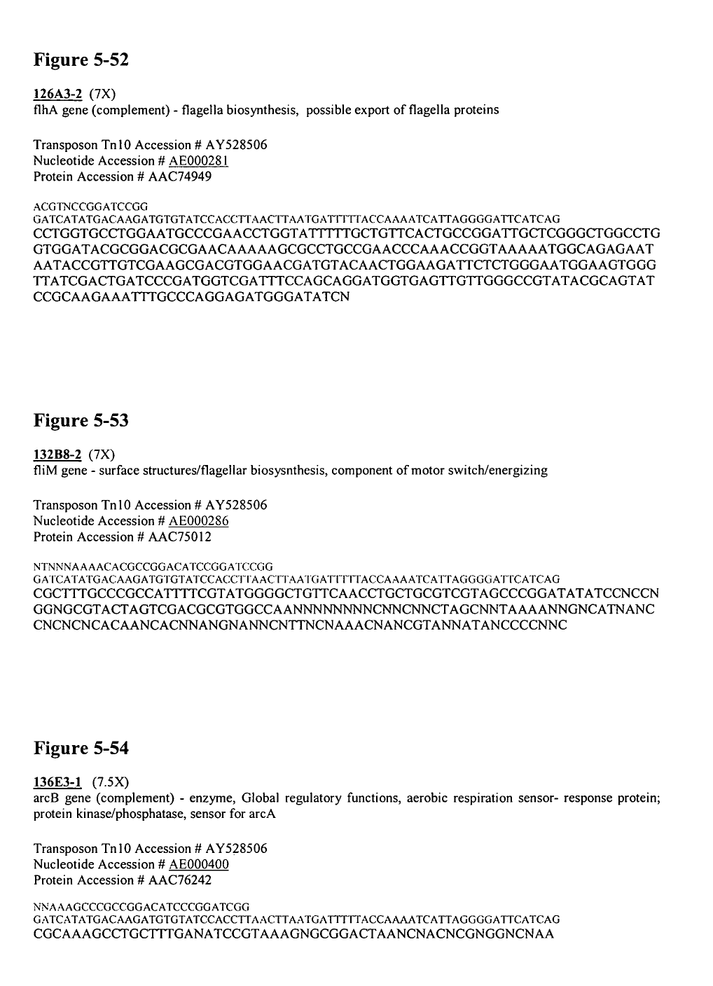

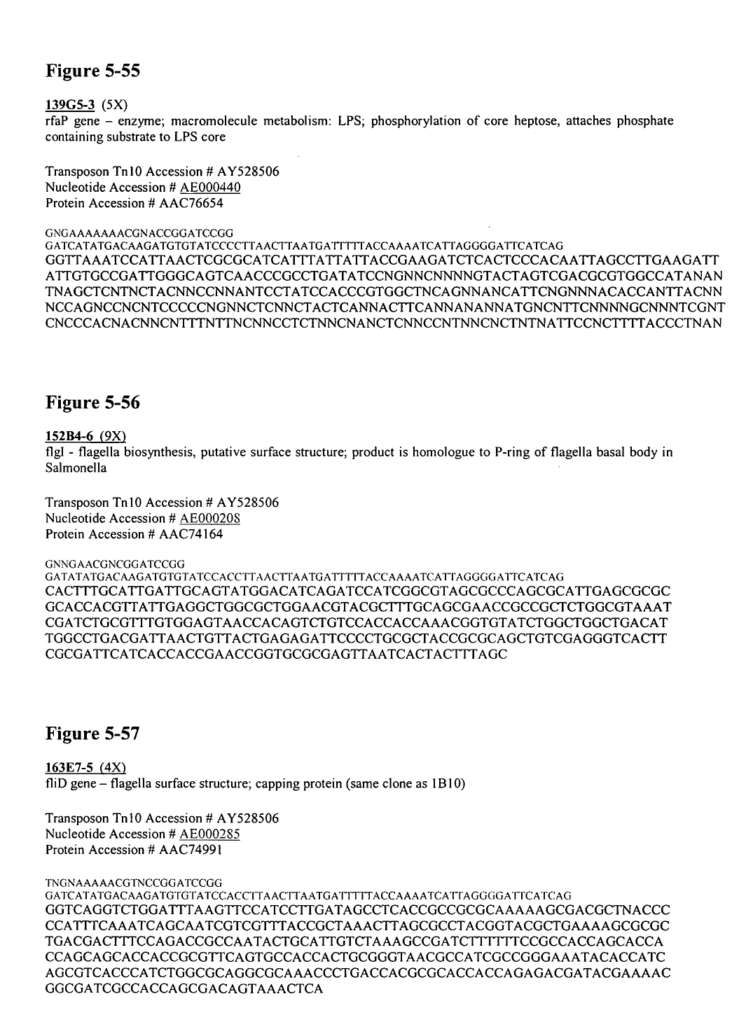

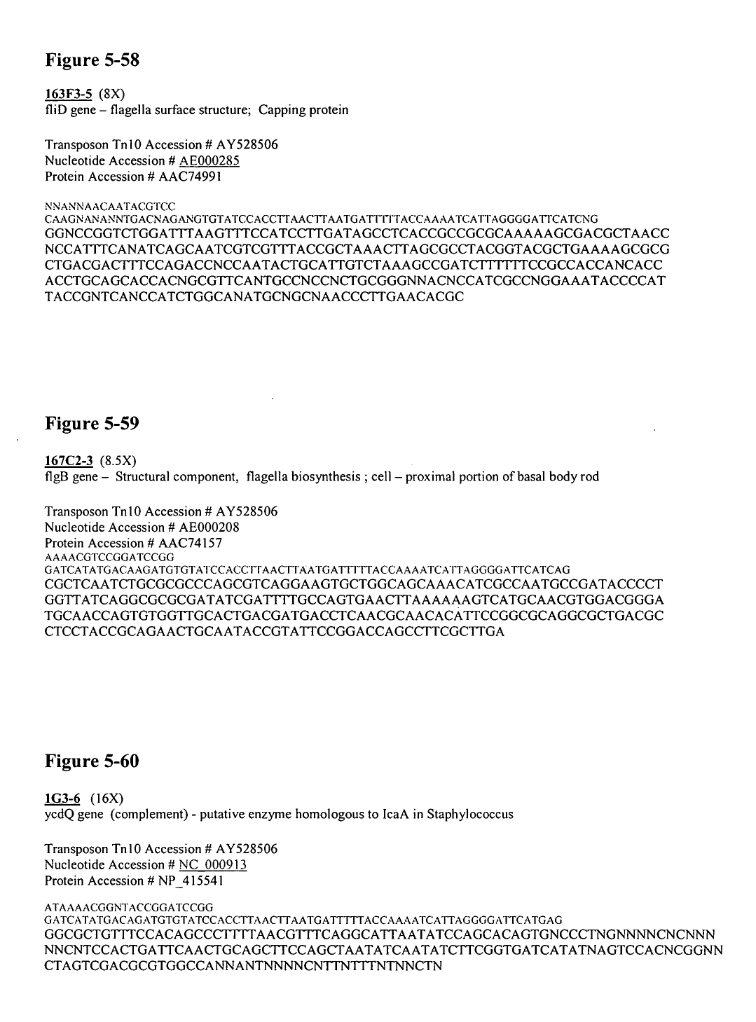

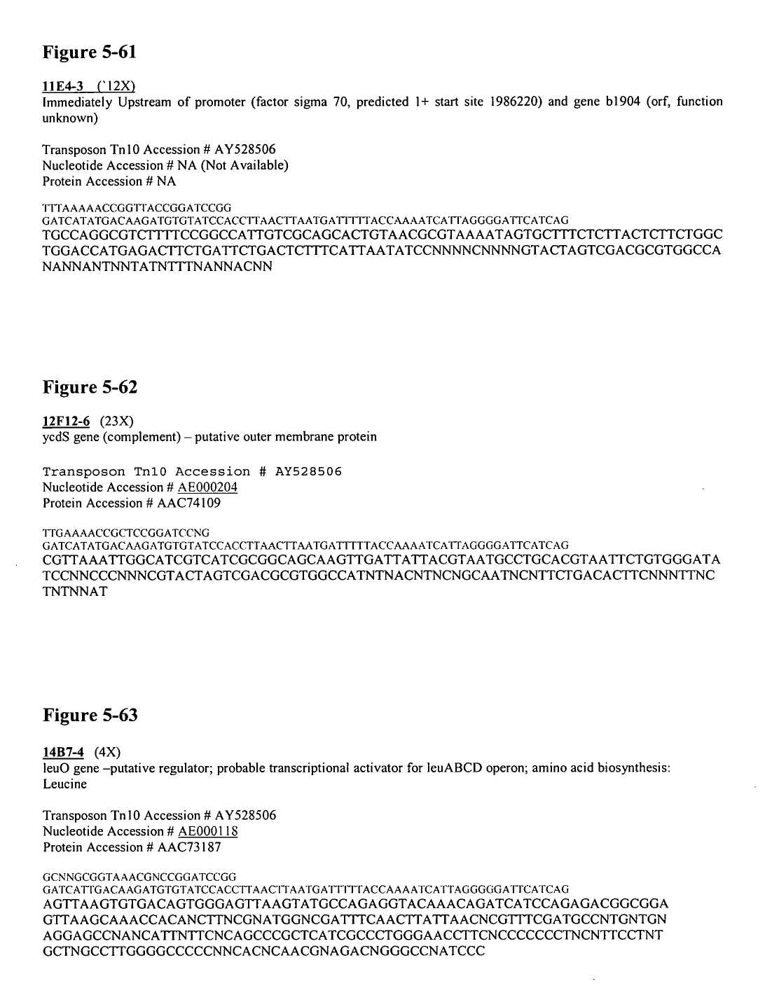

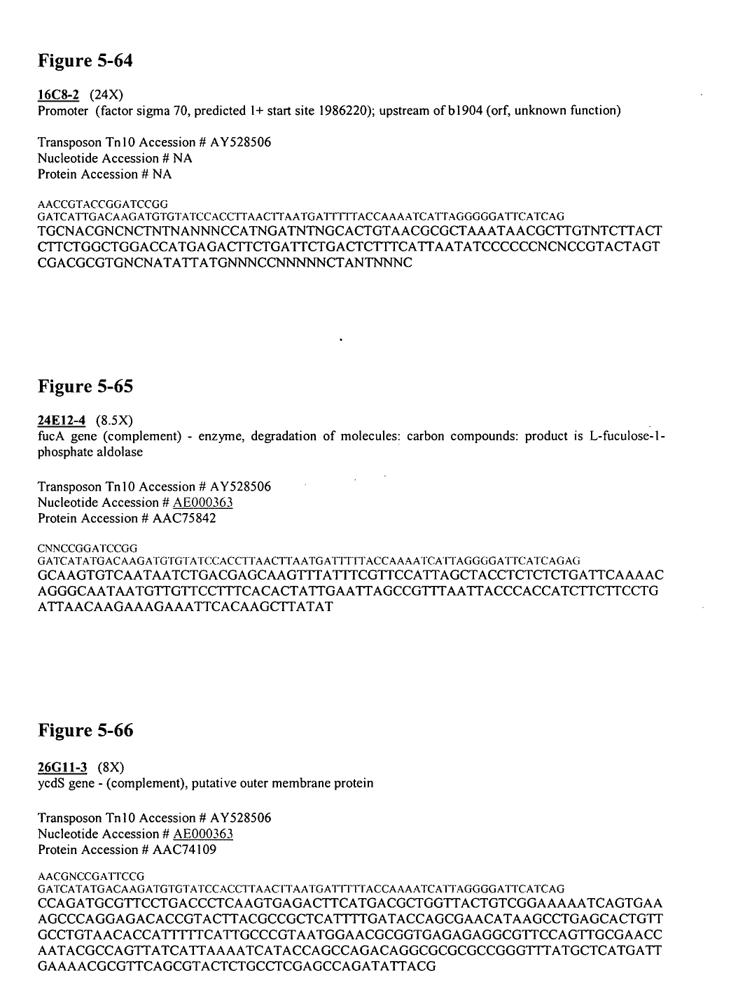

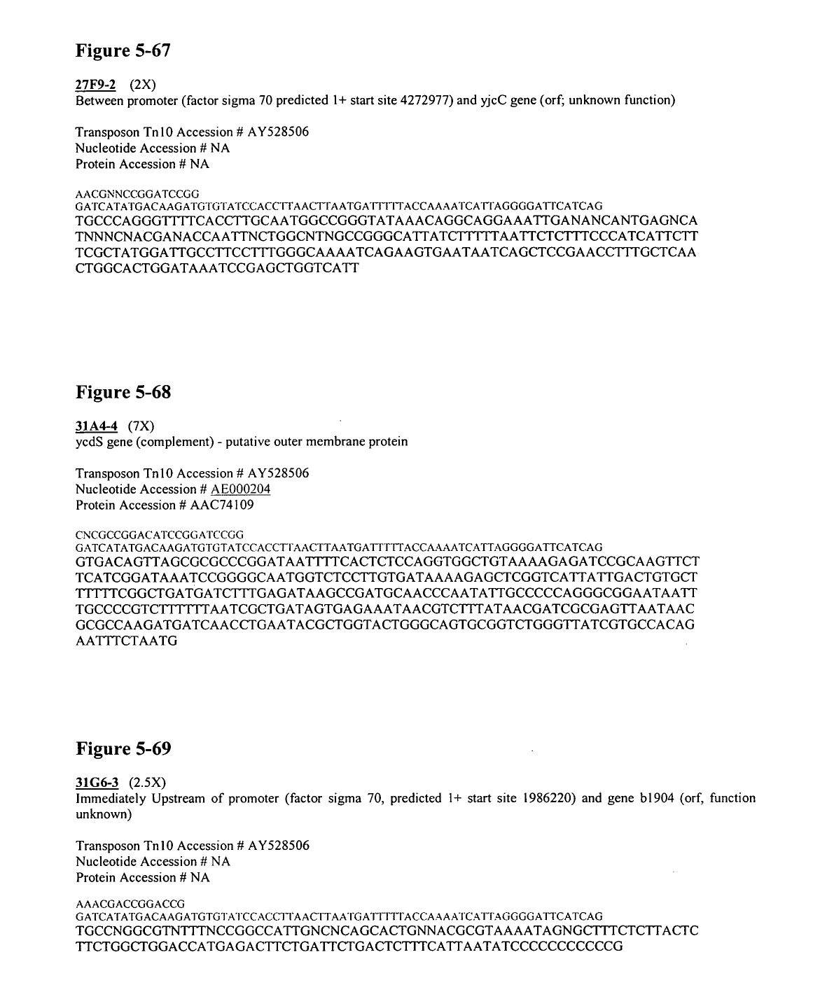

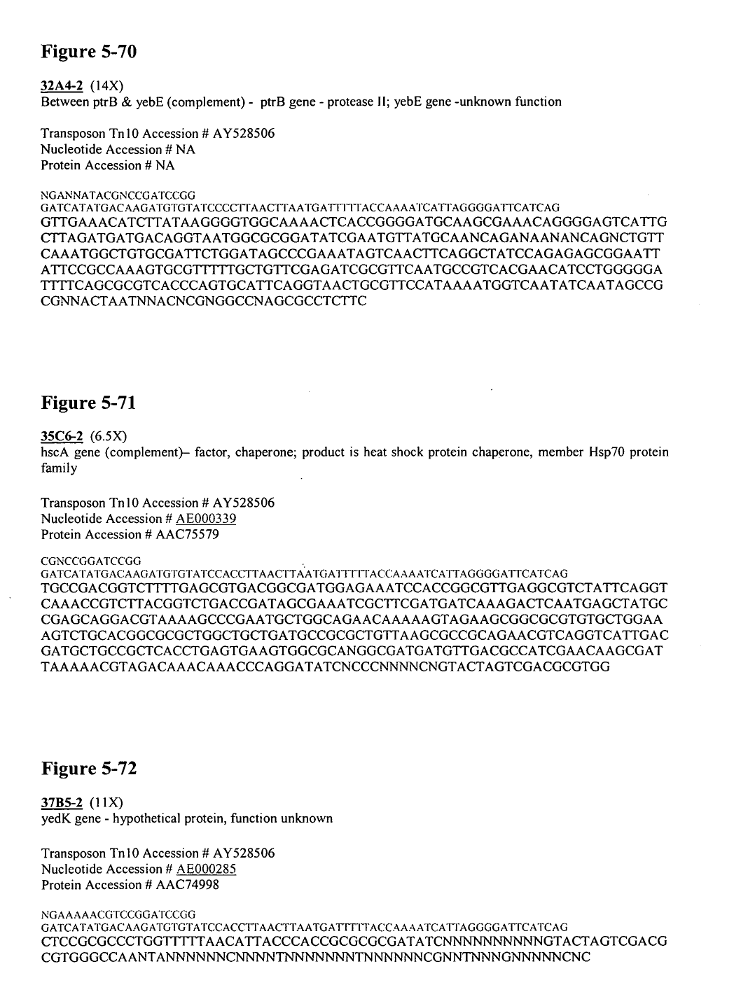

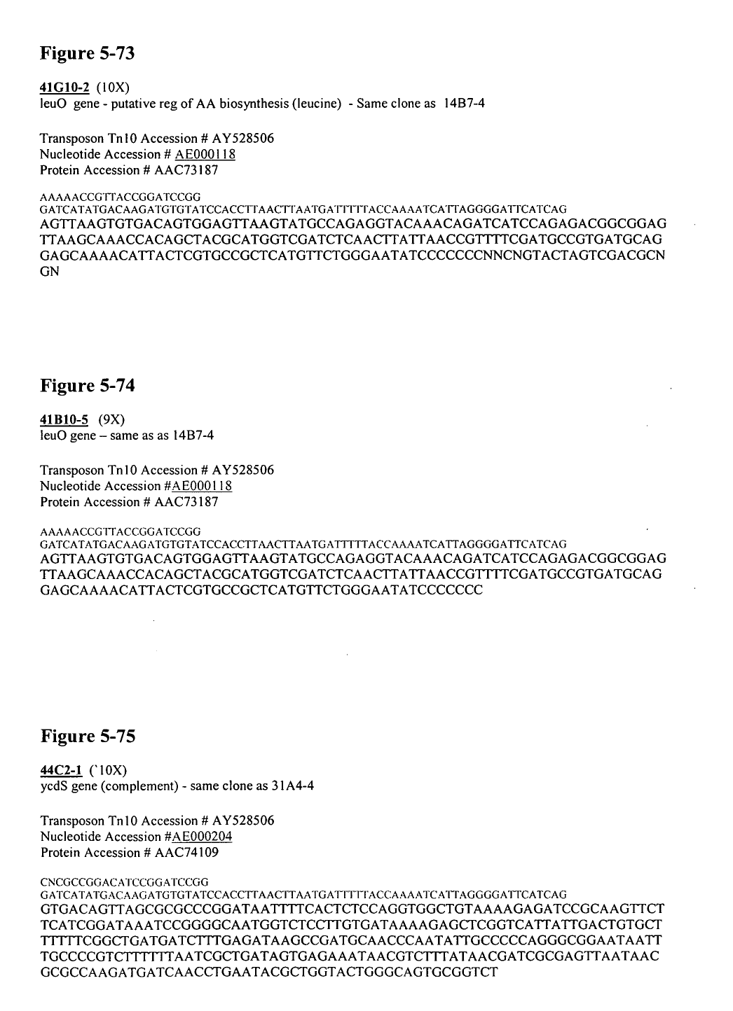

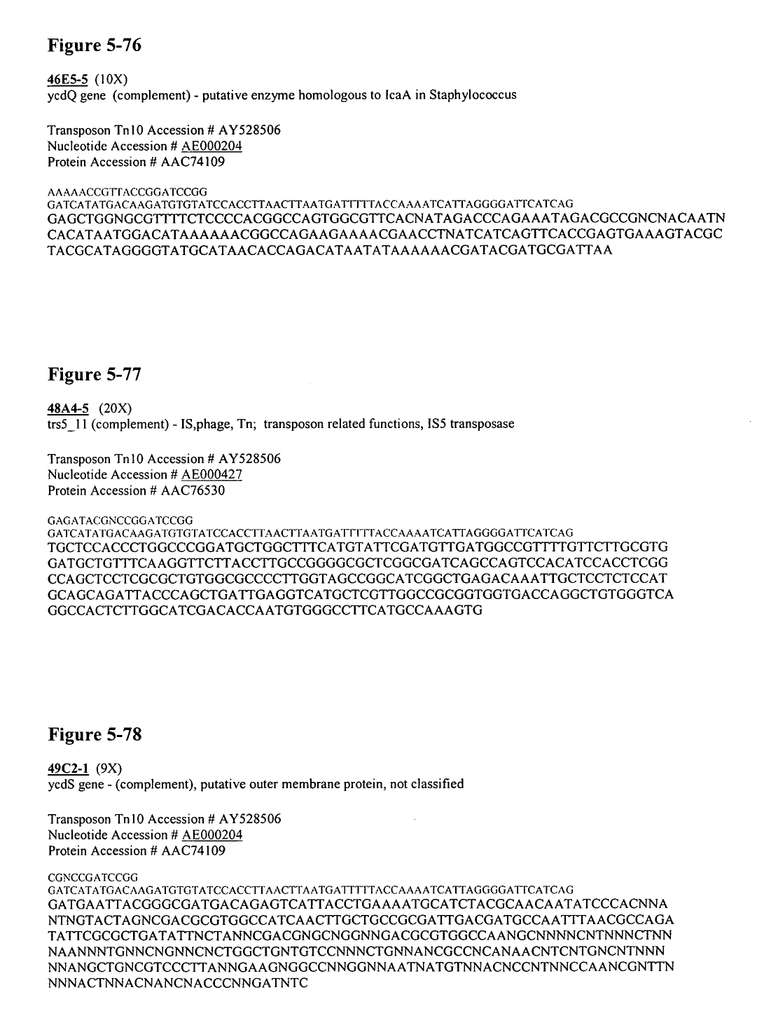

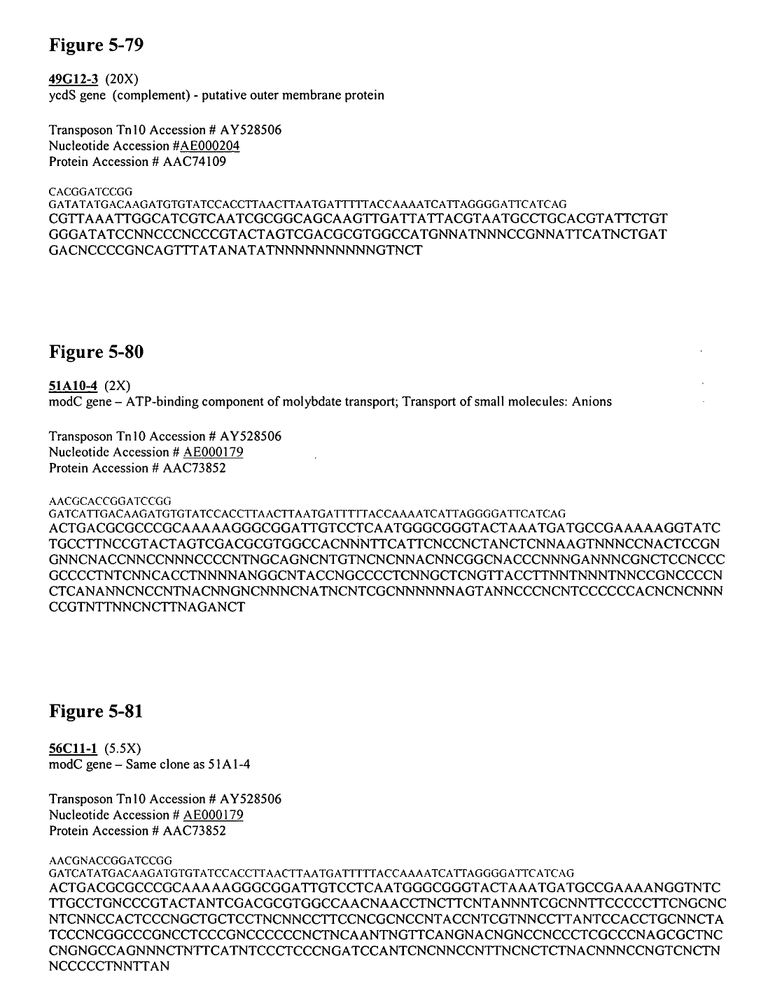

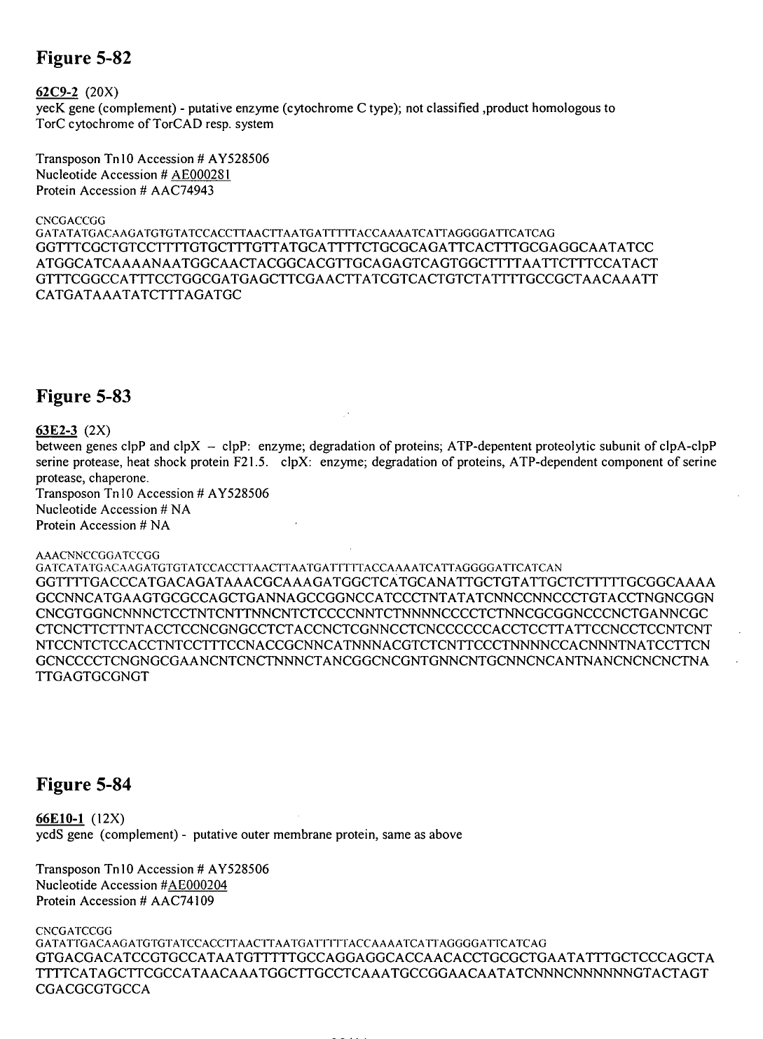

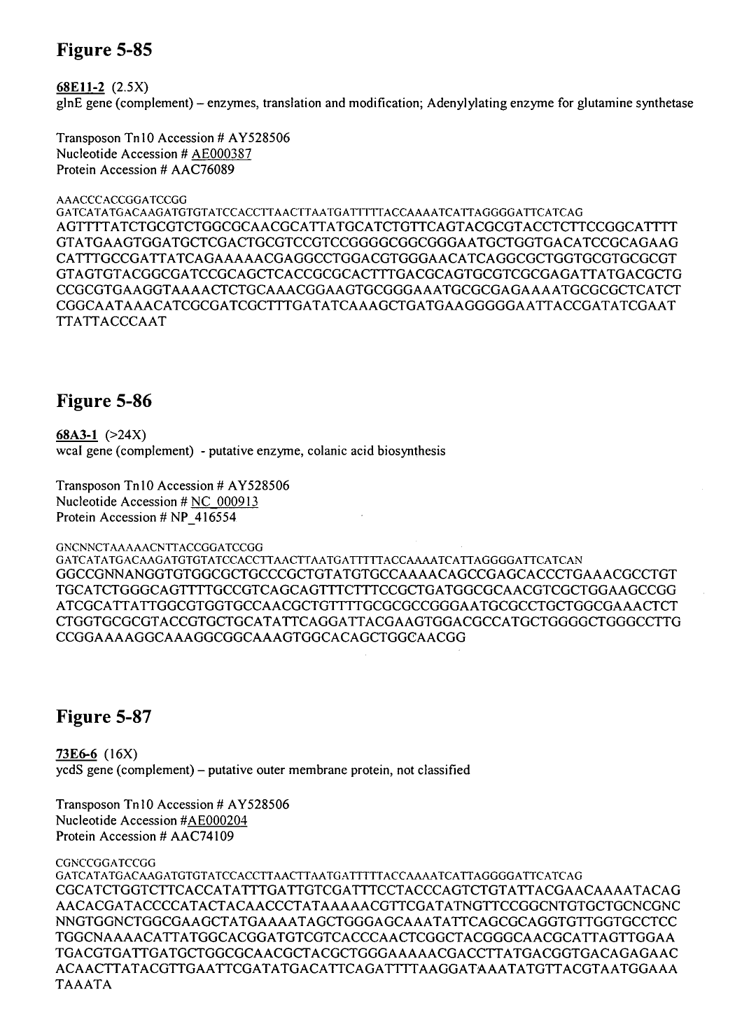

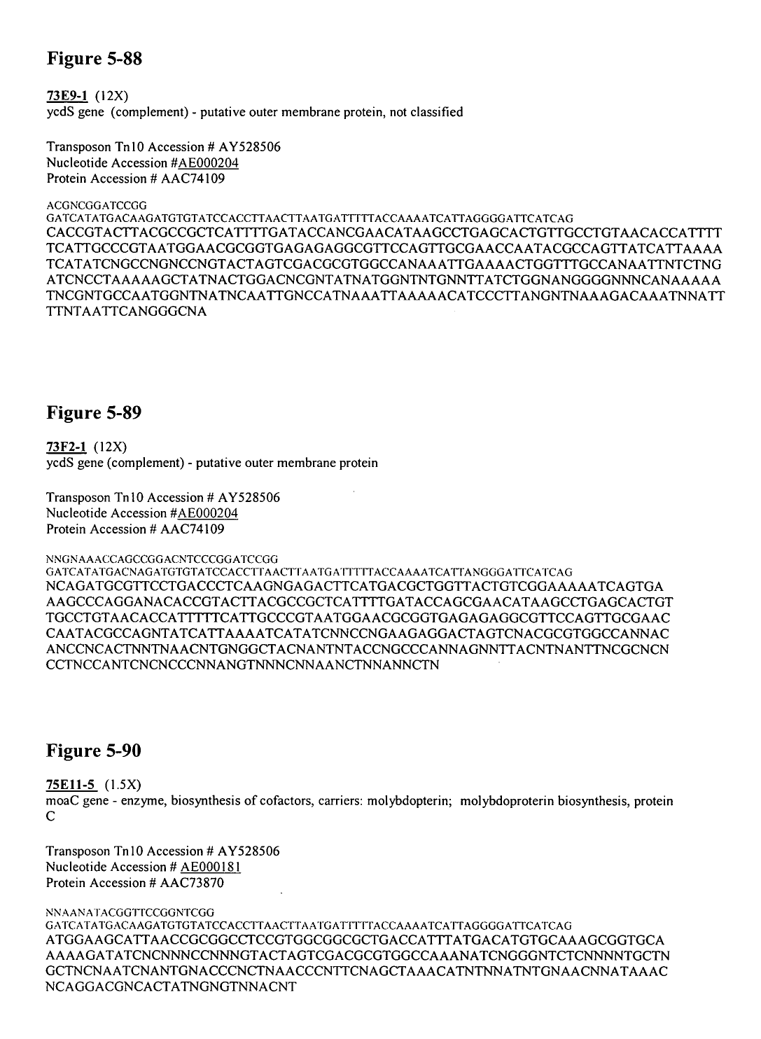

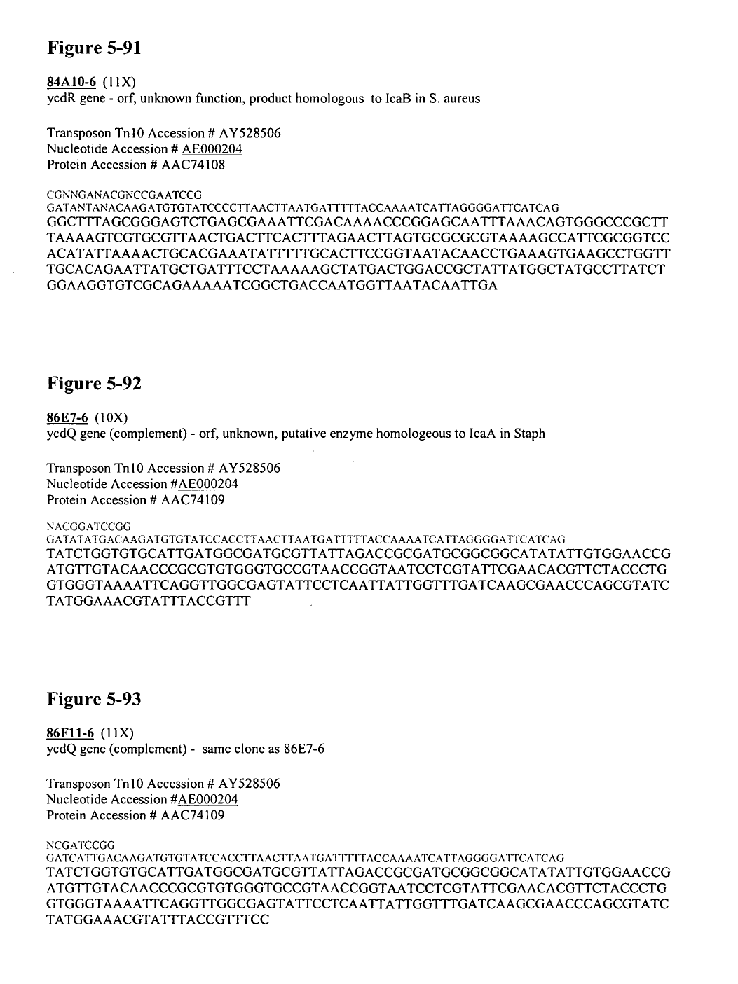

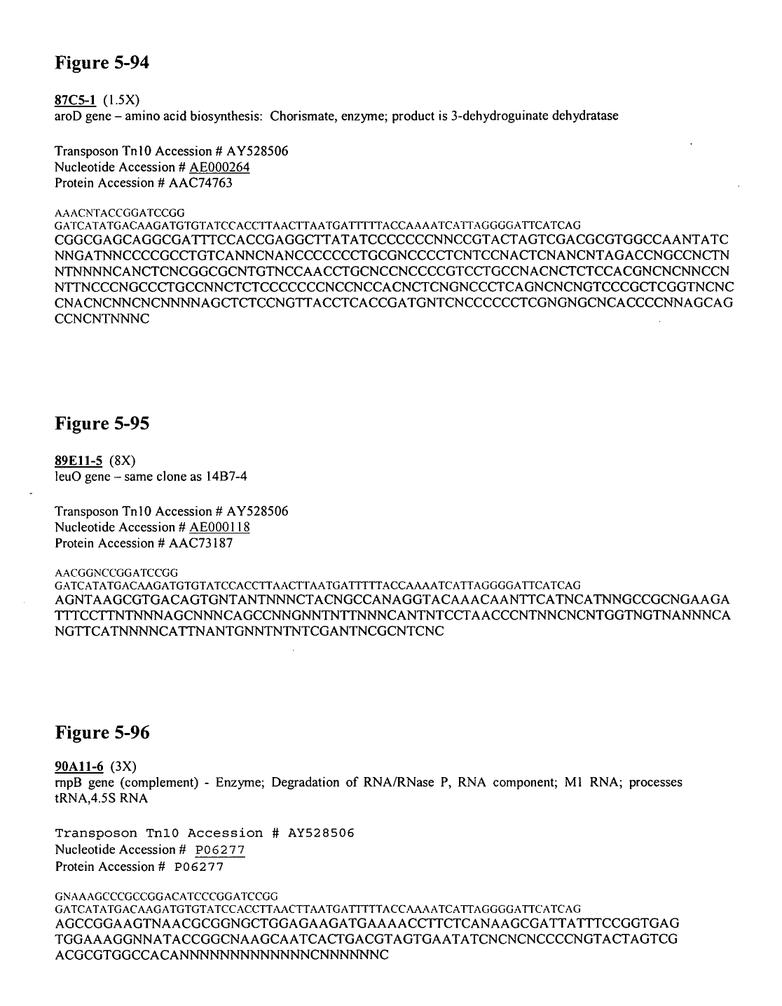

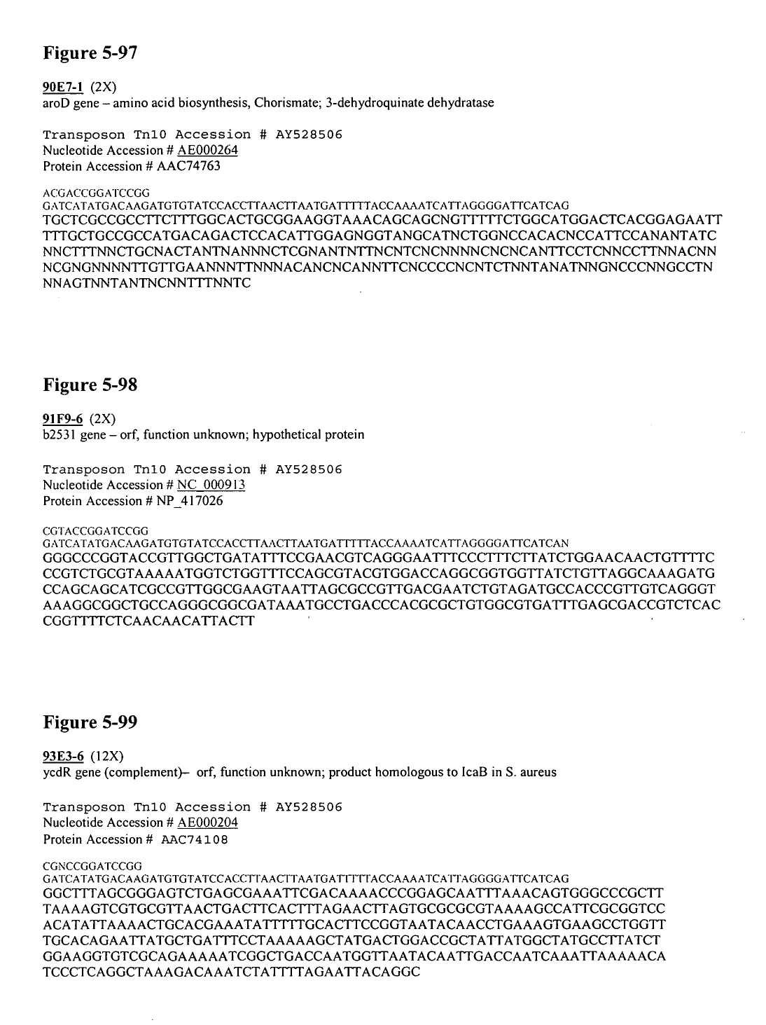

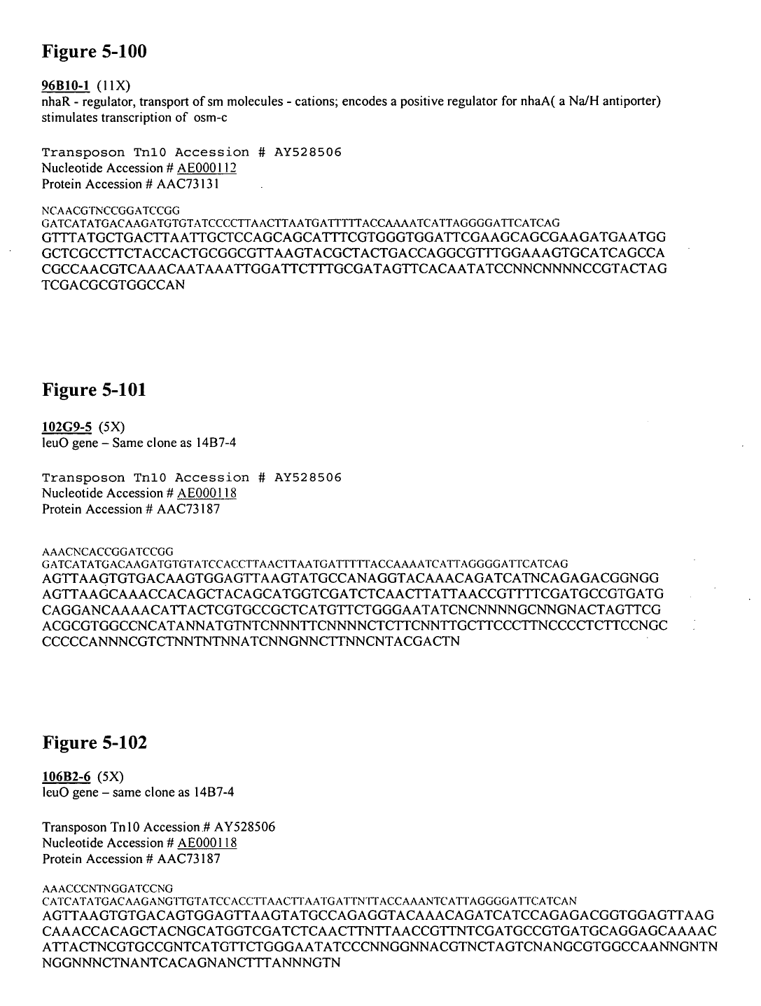

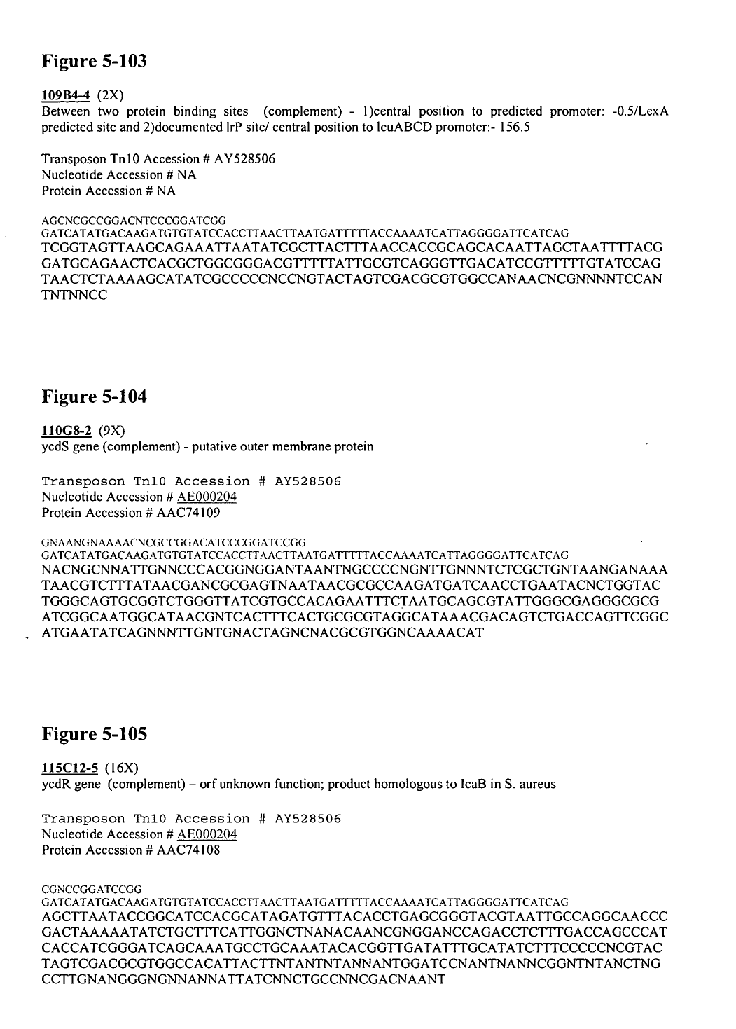

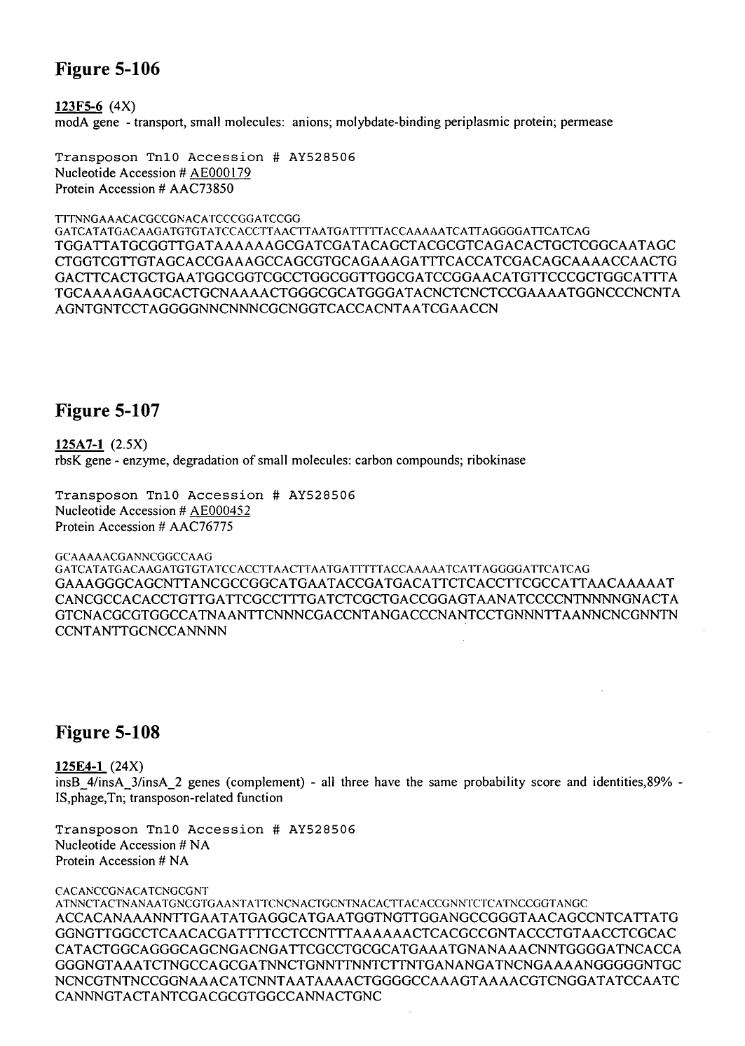

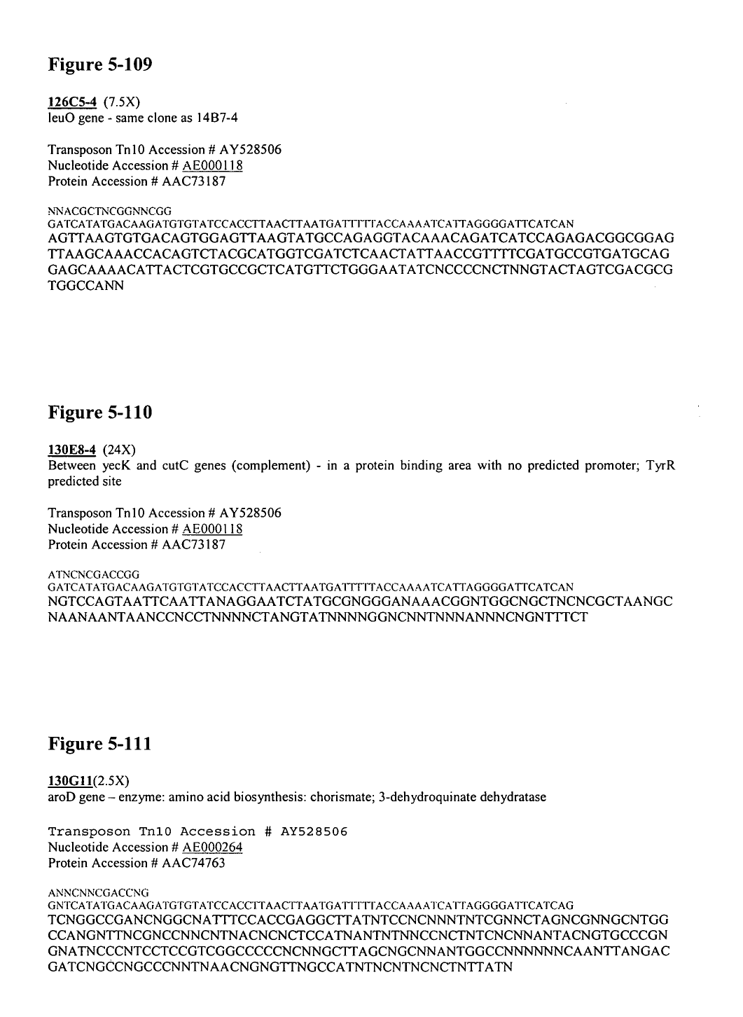

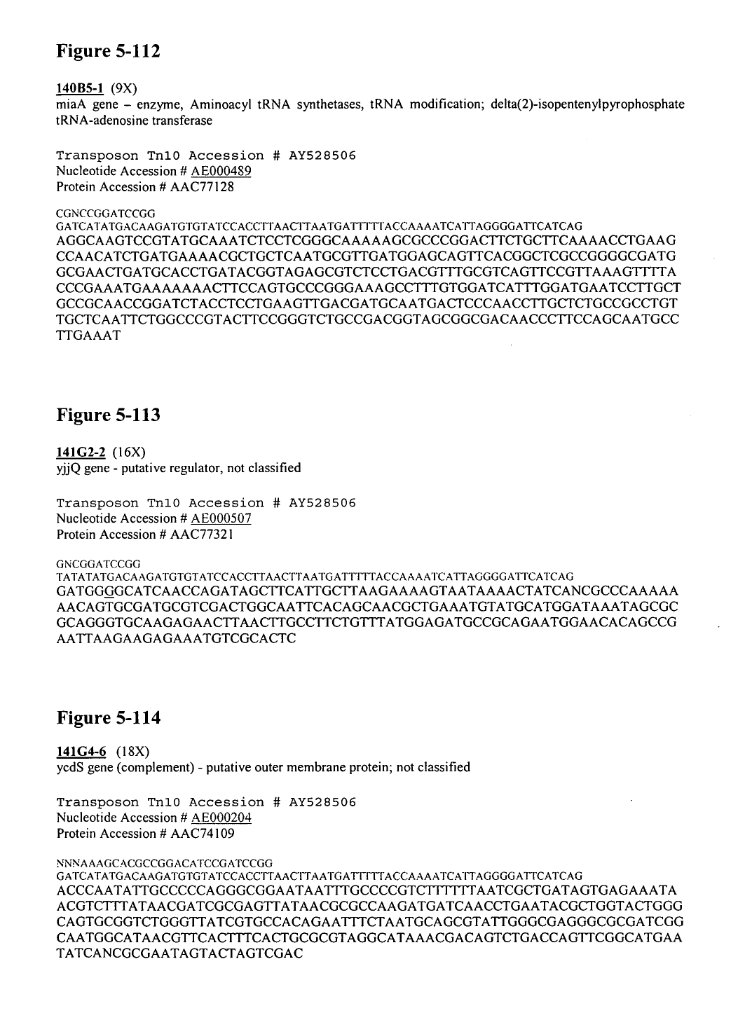

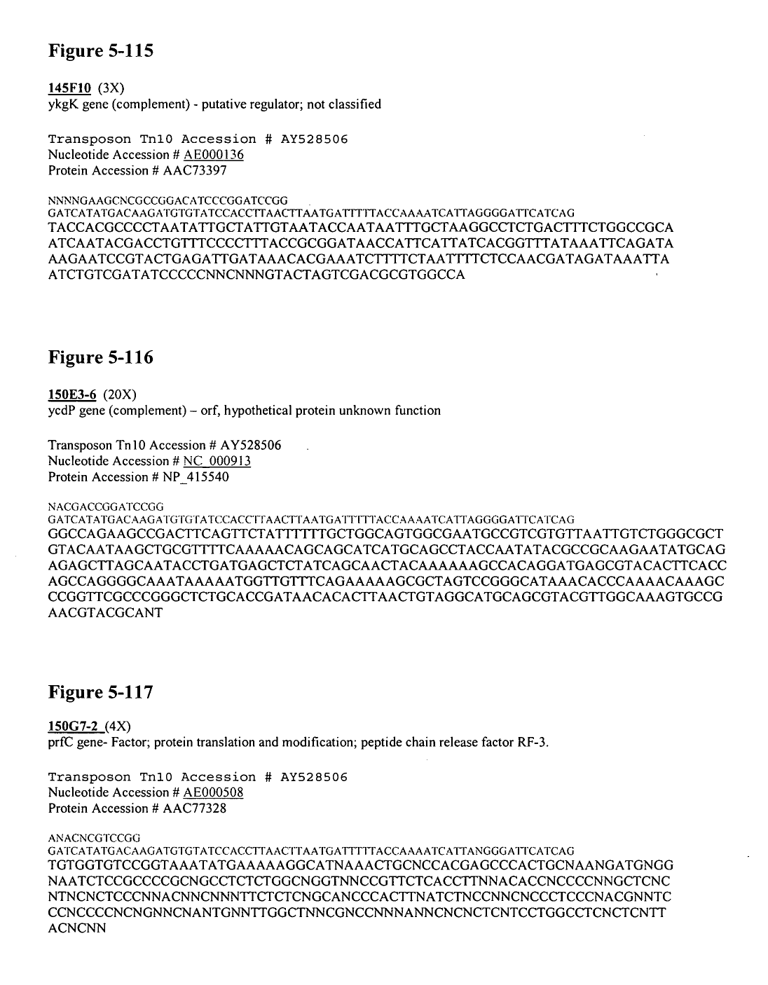

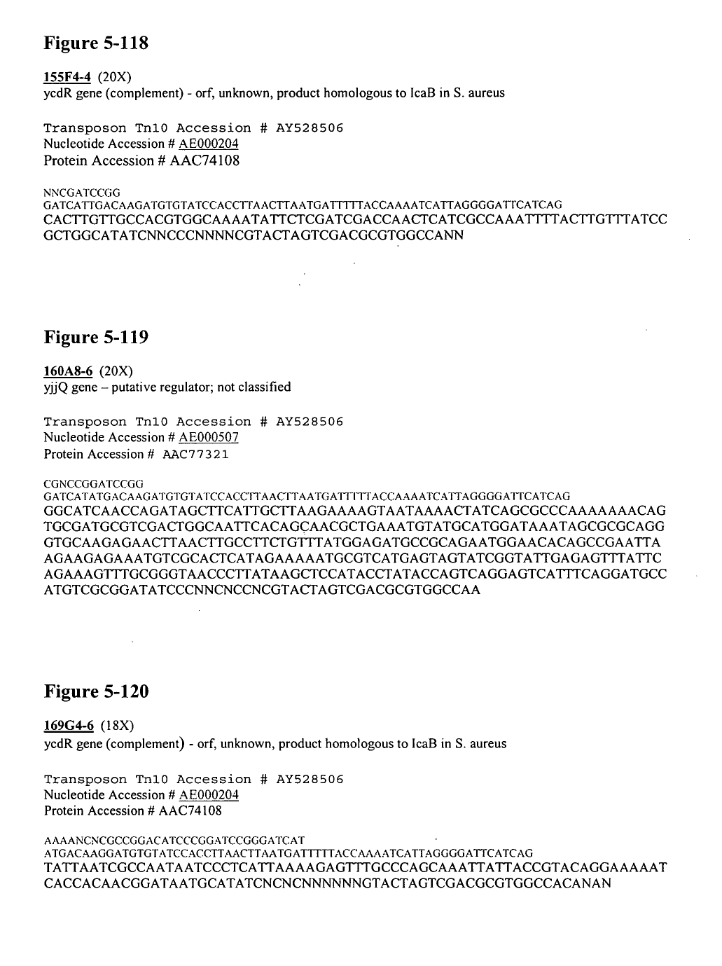

14. An isolated polynucleotide sequence, comprising a polynucleotide sequence which is selected from the group consisting of:
(a) a sequence selected from the sequences listed in FIG. 5;
(b) a sequence which is at least 90% homologous with a sequence of (a);
(d) a sequence which is at least 95% homologous with a sequence of (a);
(e) a sequence which is at least 98% homologous with a sequence of (a);
(f) a sequence which is at least 99% homologous with a sequence of (a); and;
(g) a sequence which hybridizes to any of (a) to (f) under stringent conditions.

15. A vector comprising a polynucleotide sequence of claim 14 in a suitable vector.

16. A host cell comprising a polynucleotide sequence of claim 14 in a host cell which is heterologous to said sequence.

17. A vector of claim 15, wherein said vector contains or encodes a tag.

18. A method for identifying a compound which inhibits or promotes biofilm formation, comprising the steps of:
(a) selecting a host cell of claim 3;
(b) cloning said host cell and separating said clones into a test group and a control group;
(c) treating said test group using a compound; and
(d) determining the relative quantity of an expression product of said sequence, as between said test group and said control group.

19. The method of claim 18, wherein said sequence is selected from the group consisting of strains 9B10-1, 32A4-2, 141G2-2, 68A3-1 and 141G2-2.

20. The method of claim 19, wherein said strain is 9B10-1.
